(19) United States
(12) Patent Application Publication
(10) Pub. No.: US 2020/0147149 A1
(43) Pub. Date: May 14, 2020

(54) COMPOSITIONS AND METHODS FOR THE MODULATION OF GUT SENSORY CELLS

(71) Applicants: Diego V. BOHORQUEZ, Durham, NC (US); Melanie Maya KAELBERER, Durham, NC (US); Duke University, Durham, NC (US)

(72) Inventors: Diego V. Bohórquez, Durham, NC (US); Melanie Maya Kaelberer, Durham, NC (US)

(21) Appl. No.: 16/632,062

(22) PCT Filed: Jul. 17, 2018
(86) PCT No.: PCT/US2018/042552

§ 371 Date: Jan. 17, 2020

Related U.S. Application Data
(60) Provisional application No. 62/533,252, filed on Jul. 17, 2017.
(60) Provisional application No. 62/613,153, filed on Jan. 3, 2018.
(60) Provisional application No. 62/642,001, filed on Mar. 12, 2018.
(60) Provisional application No. 62/642,809, filed on Mar. 14, 2018.

(51) Int. Cl. A61K 35/741; A61K 31/7004; A61K 31/7016; A61K 31/7034; A61K 31/47; A61K 31/662; A61K 31/5513; A61P 43/00

(52) U.S. Cl. CPC A61K 35/741; A61K 31/7004; A61K 31/7016; A61P 43/00; A61K 31/47; A61K 31/662; A61K 31/5513; A61K 31/7034

(57) ABSTRACT

The present disclosure relates to compositions and methods for modulating a transsynaptic signal through a neuroepithelial circuit between a gut sensory cell and the brain, as well as methods that involve modulating a transsynaptic signal through a neuroepithelial circuit between a gut sensory cell and the brain.

### CROSS-REFERENCE TO RELATED APPLICATIONS

This application claims the benefit of the earlier filing dates of U.S. Provisional Application No. 62/533,252, filed Jul. 17, 2017, U.S. Provisional Application No. 62/613,153, filed Jan. 3, 2018, U.S. Provisional Application No. 62/642,001, filed Mar. 12, 2018, and U.S. Provisional Application No. 62/642,809, filed Mar. 14, 2018, all of which are incorporated herein by reference in their entirety.

### GOVERNMENT FUNDING

The subject matter of this invention was made with Government support under Federal Grant Nos. KO1 DK-103832, R03 DK114500-01, P30 DK034987, and 1OT2OD023849-01 awarded by the National Institutes of Health (NIH) and Federal Grant No. N2002850300 awarded by the Defense Advanced Research Projects Agency. The Government has certain rights to this invention.

### TECHNICAL FIELD

Embodiments of the present disclosure relate generally to compositions and methods for modulating a transsynaptic signal through a neuroepithelial circuit between a gut sensory cell and the brain, as well as methods that involve modulating a transsynaptic signal through a neuroepithelial circuit between a gut sensory cell and the brain.

### BACKGROUND

The brain perceives the environment through specialized sensory neuroepithelial circuits. In the tongue, for instance, taste receptor cells transduce chemical signals by synapsing with the glossopharyngeal nerve. In the gut, however, the putative sensory epithelial cell known as the enteroendocrine cell was thought to convey signals to the nerves only through endocrine mechanisms—hence its name. There is a cohort of modern health disorders closely related to alter gut-brain signaling and it was assumed that the nerves innervating the gut only received sensory information from the gut lumen via hormones released from enteroendocrine cells.

Whereas sight, sound, scent, touch, and taste are transduced to the brain by innervated sensor cells, nutrient sensing in the gut (gut luminal sensing) is thought to be solely hormonal and take place only indirectly through paracrine mechanisms. For instance, Merkel cells of the skin synapse with somatosensory afferents to trigger the feeling of fine textures and chemosensory taste cells of the tongue synapse with the glossopharyngeal nerve to convey the flavor of sugars. These synaptic transmissions provide precise temporal and topographical representations of the stimulus required for real time efferent feedback from the nerve back to the sensor. However, in the gut, epithelial nutrient sensing has been described as endocrine because the putative sensor cell—the enteroendocrine cell—was assumed to lack synaptic contacts with the cranial nerve that processes sensory signals from the viscera—the vagus nerve. The dogma is revealed in the enteroendocrine cell's name, a term that is rooted in the notion that nutrients stimulate the release of hormones, which either enter the bloodstream or act on nearby nerves minutes to hours post-ingestion. Research has been focused of gut-brain signaling evolved around the bowel's ability to modulate eating behaviors by sensing nutrients and secreting hormones. In fact, most, perhaps all, of the classical gut hormones associated with enteroendocrine cells—Cholecystokinin (CCK), Peptide YY (PYY), Ghrelin, Glucagon-like peptide 1, Amylin, Glucose-dependent insulinotropic peptide, Substance P, Serotonin, etc. have been linked to the regulation of appetitive behaviors.

Enteroendocrine cells also have conserved features of sensory epithelial transducers, including synapses. For instance, they express mechanical, olfactory, and taste receptors. They also contain voltage-gated ion channels that render them electrically excitable and are capable of forming synapses. Almost two-thirds of enteroendocrine cells synapse with nerves innervating the small and large intestinal mucosa. Similar findings have been reported recently in a subset of enteroendocrine cells often referred to as enterochromaffin cells. Though the origin of those nerves was unclear at the time. Gastrointestinal chemosensation has been studied so far from an endocrine perspective. The reason is that enteroendocrine cells, the sensory epithelial cells of the gut, were thought to communicate with nerves only indirectly, through hormones.

The luminal contents of the gut influence brain function and behavior. Nutrients based on their calorie load modulate hypothalamic neurons controlling food intake within seconds of entering the intestine. In fact, even in the absence of taste signaling, glucose entering the duodenum drives learning by stimulating dopamine-dependent reward in the brain basal ganglia. Despite the established effects of gastrointestinal sensing on brain function, the neural circuits transducing such information to the brain from the gut lumen remains undocumented. In recent years, enteroendocrine cells have emerged as sensors of mechanical, chemical and bacterial signals in the gastrointestinal tract

Autism is a neurodevelopmental disorder characterized by social impairments, communication difficulties and repetitive/stereotyped behaviors. While there is a substantial genetic component in autism, there is also evidence that environmental factors can contribute to the complex pathogenesis of autism spectrum disorders (ASD). The core symptoms of autism are deficits in social interaction and language, and the presence of repetitive/stereotyped behaviors. Autism spectrum disorder (ASD) affects 1 in 68 children born in the US today. Early in life, children diagnosed with ASD have trouble socializing, develop repetitive behaviors, and most suffer from gastrointestinal (GI) disturbances, that range from chronic constipation or diarrhea, to inflammatory or irritable bowel symptoms. ASD is a major public health challenge and there are currently no effective drug therapies that address the core neurological symptoms of this disorder. Thus, there is an unmet need for treating disorders such as eating disorders, ASD and anxiety, as well as an unmet need for modulating nutrient sensing and eating behaviors in subjects.

### SUMMARY

The present disclosure is directed to a method of modulating a transsynaptic signal through a neuroepithelial circuit between a gut sensory cell and the brain. The method includes stimulating or inhibiting a receptor on the gut sensory cell, thereby stimulating or inhibiting the transsynaptic signal from the gut sensory cell to the brain.

The present disclosure is directed to a method of modulating a caloric value of a nutrient to a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain and modulating the caloric value of the nutrient to the subject.

The present disclosure is directed to a method of modulating a bacterial stimulus signal in a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain and modulating the bacterial stimulus signal in the subject.

The present disclosure is directed to a method of modulating food intake behavior and/or food preference in a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the food intake and/or food preference behavior in a subject is modulated.

The present disclosure is directed to a method of treating a subject having or suspected of having an eating disorder. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein eating behavior of the subject is modulated and the subject is treated.

The present disclosure is directed to a method of modulating anxiety in a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the anxiety of the subject is modulated.

The present disclosure is directed to a method of treating autism spectrum disorders in a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the eating behavior of the subject is modulated and the subject is treated for autism.

### BRIEF DESCRIPTION OF THE DRAWINGS

FIGS. 1A-1E show enteroendocrine cells contact sensory nerve fibers. FIG. 1A shows that CckGFP Pgp9.5GFP mice expressed GFP in CCK enteroendocrine cells and Pgp9.5 sensory nerve fibers. FIG. 1B shows a proximal small intestine villus with a GFP labeled CCK enteroendocrine cell and GFP labeled Pgp9.5 nerve fibers; 18.9%±2.0% of CckGFP cells contact Pgp9.5 fibers; N=3 mice; n=>100 cells. Bars=10 μm. FIG. 1C shows Pyy stained enteroendocrine cells (left—green) in the colon in contact with Phox2b vagal nerve fibers (center—red) in a Phox2bCRE_tdTomato mouse. Bars=10 μm. FIG. 1D shows two thirds of CCK enteroendocrine cells co-localize with presynaptic marker Synapsin-1 (n=6; 200 cells per n; Bars=10 μm). FIG. 1E real-time qPCR expression levels of presynaptic transcripts, including genes encoding for synaptic adhesion proteins (n=3; mean±S.E.M.).

FIGS. 2A-2F show enteroendocrine cells of the colon and small intestine synapse with vagal nodose neurons. FIG. 2A illustrates the mechanism of infection of enteroendocrine cells with ΔG-rabies-GFP (the overall model of ΔG-rabies-GFP enema delivery). FIG. 2B shows PYY cells expressing tdTomato (left—red) were infected by ΔG-rabies-GFP (right—green). Overlay shows 87.8% overlap±S.E.M. 2.4% (n=7 mice). In the absence of G glycoprotein (ΔG), ΔG-rabies-GFP does not spread beyond infected PYY cell. Bars=10 μm. FIG. 2C illustrates EnvA-ΔG-rabies-GFP virus entered cells via the TVA receptor and spreads using the RabG protein within specific cells. FIG. 2D shows EnvA-ΔG-rabies-GFP (right—green) infected PYY cells (left—red) and spread synaptically to underlying colon nerve fibers. 3D reconstruction of infected PYY cells and mono-synaptically labeled nerve fiber (bottom). Bars=10 μm. FIG. 2E shows EnvA-ΔG-rabies-GFP enema infected enteroendocrine cells and spread onto vagal neurons in the nodose ganglion (green). Bars=10 μm. FIG. 2F shows ΔG-rabies-GFP delivered by oral gavage in the intestinal lumen of CckCRE_rabG-TvA mice spread to label the nucleus tractus solitarious (green). This neuroepithelial circuit linked the intestinal lumen with the brain stem. Bars=10 μm.

FIGS. 3A-3H show enteroendocrine cells transduce glucose stimuli onto vagal neurons. FIG. 3A illustrates intraluminal nutrient perfusion while recording the cervical vagus activity in vivo (model of intestinal intraluminal perfusion and vagal nerve electrophysiology). FIG. 3B shows normalized traces for 300 mM sucrose, 300 mM sucrose with 3 mM phloridzin, and baseline in wild-type mice. FIG. 3C shows 300 mM sucrose and D-glucose-150 mM stimulate vagal firing rate, which was abolished by SGLT1 blocker phloridzin (n≥5, shades and error bars=S.E.M). FIG. 3D shows intestinal epithelial cells express Sglt1, but nodose neurons do not (n=3, mean±S.E.M.). FIG. 3E shows nodose neurons cultured alone (image, left) for electrophysiology (model, right). Bars=10 μm. FIG. 3F shows neuron response to a 10 mM glucose stimuli in voltage-clamp trace (left), or current-clamp trace (right). Inserts show neurons were still electrically excitable by a voltage or current pulse. FIG. 3G shows nodose neurons co-cultured with GFP+ enteroendocrine cells (image-left) for electrophysiology (model-right). (Images-bottom) Innervated enteroendocrine cells. Bars=10 μm. FIG. 3H shows in co-cultures glucose evoked EPSCs (top-left) and action potentials (top-right) in connected neurons. Quantification of EPSC amplitude, frequency (bottom left and center) (n=21 alone; n=6 pairs), and action potentials (bottom right) (n=21 alone; n=5 pairs).

FIGS. 4A-4N show milliseconds transduction and glutamate as candidate neurotransmitter. FIG. 4A illustrates intraluminal excitatory laser stimulation applied while recording the cervical vagus (model of intraluminal photo-stimulation and vagal electrophysiology). FIG. 4B shows Cck antibody co-localizes with tdTomato+ enteroendocrine cells in small intestine (SI) of CckCRE_ChR2 mice. In CckCRE_ChR2-tdTomato mice, intestinal enteroendocrine cells express channelrhodopsin (ChR2). FIG. 4C shows normalized traces for 473 nm intraluminal laser, sucrose 300 mM, and baseline in CckCRE_ChR2 mice. FIG. 4D shows 473 nm intraluminal laser stimulation of vagal activity in CckCRE_ChR2 but not wild-type mice. FIG. 4E shows patch-clamp electrophysiology of neurons (model-left) in co-culture with CckCRE_ChR2 cells (image-right). FIG. 4F shows in co-cultures, 470 nm photo-stimulation evoked EPSCs (trace-left) in connected nodose neurons (quantification-right). FIG. 4G shows inhibitory laser stimulation during nutrient perfusion while recording the cervical vagus (model of luminal photo-inhibition and vagal electrophysiology). FIG. 4H shows Cck antibody co-localization with YFP+ enteroendocrine cells in small intestine (SI) of CckCRE_Halo mice. In CckCRE_Halo-YFP mice, intestinal enteroendocrine cells express halorhodopsin (NpHR3.0). FIG. 4I shows normalized traces for baseline, sucrose 300 mM, sucrose 300 mM+532 nm intraluminal laser. FIG. 4J shows laser inhibits sucrose activity in CckCRE_Halo but not wild-type mice. In CckCRE_Halo but not wild-type mice, 532 nm intraluminal laser abolishes sucrose effect on vagal firing rate. FIG. 4K shows enteroendocrine cells express VGLUT1 (Slc17a 7). FIG. 4L shows CCK antibody co-localization with YFP+ Vglut1 cells in small intestine Vglut1CRE_ChR2-YFP mice. FIG. 4M shows normalized traces for baseline, sucrose 300 mM, and 473 nm intraluminal laser in Vglut1CRE_ChR2-YFP mice. FIG. 4N shows 473 nm laser stimulation of vagal activity in Vglut1CRE_ChR2-YFP but not wild-type mice. n≥5 per group, error bars and shaded regions indicate mean±S.E.M.

FIGS. 5A-5B shows single cell Western blot of duodenal enteroendocrine cells. FIG. 5A shows Western Blot analysis of single CCK GFP cells probed with Rabbit anti-Synapsin-1 and Chicken anti-GFP antibody. Fluorescence intensity was measured and the area under the peak intensity curve was calculated. FIG. 5B shows the peak area from FIG. 5A corresponded to the amount of protein detected. 164 cells of 198 GFP positive cells (83%) were also positive for Synapsin-1.

FIGS. 6A-6D shows in vitro rabies transfection and retro-tracing in intestinal organoids co-cultured with vagal nodose neurons. FIG. 6A shows CckCRE_tdTomato organoids grown in culture and transfected with ΔG-rabies-GFP. Rabies transduced CCK cells, as shown by endogenous fluorescence and antibody staining. Bars=10 μm. FIG. 6B shows that in a co-culture of PGP9.5-GFP nodose neurons with CckCRE_tdTomato organoids neurons grew out and made contact with CCK cells in organoids. Arrow indicates point of contact. Bars=10 μm. FIG. 6C shows that wild-type nodose neurons transduced by ΔG-rabies-mCherry were not able to infect synaptically connected cells. Bars=10 μm. FIG. 6D shows Phox2bCRE_rabG-Tva neurons transduced with EnvA-ΔG-rabies-mCherry. Rabies was able to mono-synaptically trace and transduce connected enteroendocrine cells. Bars=10 μm.

FIGS. 7A-7D show Fast Blue enema labeled vagal neurons innervating the colon. FIG. 7A illustrates Fast Blue labeling of vagal neurons innervate the colon and was transported vagal ganglia. Fast Blue was actively transported from the colon to the vagal ganglia. FIG. 7B shows a colon cross section of wild-type mice with dye was present in the distal colon only. FIG. 7C shows Fast Blue absorption in enteric neurons. FIG. 7D shows labeled neurons in the vagal nodose ganglion. (n=4).

FIGS. 8A-8F show monosynaptic rabies tracing from the intestine to sensory ganglia. FIG. 8A illustrates a colon enema of EnvA-ΔG-rabies-GFP performed to trace monosynaptic connections. FIG. 8B shows a colon enema of EnvA-ΔG-rabies-GFP in PyyCRE_RabG-Tva colon with PYY antibody staining, 5 out of 9 mice were transduced. FIG. 8C shows a control mice with no transduction in dorsal root ganglion control (n=4 control mice). FIG. 8D shows a dorsal root ganglion in 4 out of 5 colon transduced mice with rabies transduction of GFP. FIG. 8E shows a gavage of ΔG-rabies-GFP in control mouse nodose (n=1) with no transduction. FIG. 8F shows a gavage of ΔG-rabies-GFP in CckCRE_RabG-Tva mice nodose (n=2 of 3 mice) with rabies transduction in the nodose ganglia.

FIG. 9 shows a sub-diaphragmatic vagotomy attenuates the increase in vagal firing rate from sucrose and laser. Normalized maximum firing rate for 300 mM sucrose in wild-type mice and intraluminal 473 nm laser in CckCRE_ChR2 mice before (−) and after (+) sub-diaphragmatic vagotomy (N≥3, error bars indicate S.E.M.). Stars indicate significant difference (p<0.05) using ANOVA and Tukey HSD post hoc analysis.

FIG. 10 shows that high osmolarity PBS (700 mOsm) delivered intraluminally and 300 mM sucrose delivered intra-peritoneally did not increase vagal firing rate. Normalized maximum firing rate for 300 mM sucrose intraluminal, PBS (700 mOsm) intraluminal, and 300 mM sucrose IP in wild-type mice (N≥3, error bars indicate S.E.M.). Star indicates significant difference (p<0.05) using ANOVA and Tukey HSD post hoc analysis.

FIG. 11 shows the vagal firing rate responds to varying sucrose concentrations. Normalized maximum firing rate for 3 mM, 30 mM, 100 mM, and 300 mM sucrose in wild-type mice (N≥3, error bars indicate S.E.M.). Stars indicate significant difference (p<0.05) using ANOVA and Tukey HSD post hoc analysis.

FIGS. 12A-12C show D-glucose does elicit a calcium response in enteroendocrine cells. FIG. 12A shows organoids from CckCRE_tdTomato mice transduced with ΔG-rabies-GCaMP6s and imaged using multiphoton microscopy. Cells were given an experimental sugar stimulus (10 mM sucrose, or 20 mM D-glucose), followed by a wash and a 40 mM KCl control stimuli. FIG. 12B shows the fluorescence traces of individual enteroendocrine cells-grey, and the average trace across cells-black. FIG. 12C shows the magnitude of the sugar response calculated as the percent fluorescence of the KCl response. The sucrose response was calculated to be 72.4±10.6% of KCl (n=5), and the D-glucose response was calculated to be 56.0±20% of the KCl response (n=3).

FIGS. 13A-13C show dextrose did not elicit a calcium response in nodose neurons. FIG. 13A shows dissociated Phox2bCRE_GCaMP6s nodose neurons plated for in vitro calcium imaging. An experimental D-glucose (20 mM) stimuli was applied followed by a wash then KCl (40 mM) as a control stimulus. FIG. 13B shows (left) fluorescence traces of cells-grey, and the average trace across cells-black of nodose neurons imaged. 2.7±1.9% responded to glucose and 97.3±1.9% of neurons did not respond to 20 mM D-glucose. (mice=3, cells=246, error bars indicate S.E.M., * student's t-test determined p=3.7E-06). FIG. 13C shows nodose neurons that innervated the small intestine identified by Fast Blue tracing from the proximal duodenum. Calcium imaging of Fast Blue positive neurons showed 100% of neurons did not respond to a 20 mM D-glucose stimulus. (mice=2, cells=73). Bars=10 μm.

FIGS. 14A-14B show enteroendocrine cells in CckCRE_ChR2-tdTomato mice decreased mouse eating behavior when activated and were light sensitive. FIG. 14A shows CckCRE_ChR2-tdTomato mice implanted with an abdominal window (top). They were fasted overnight then 473 nm laser stimulation was administered through the window for 30 minutes. After stimulation, the number of food pellets cumulatively eaten was measured. CckCRE_ChR2-tdTomato mice ate significantly less than control wild type mice (n=3 experimental, n=5 control, significance was determined using a two-tailed Student's t test). FIG. 14B shows recoding from CckCRE_ChR2-tdTomato mice using extracellular sharp electrode recording in gut sections. 560 nm light did not elicit activity, whereas, 470 nm light elicited did elicit a response.

FIG. 15A shows vagal firing rate response to laser application at sub-diaphragmatic or cervical vagus in CckCRE_ChR2-tdTomato mice. Normalized maximum firing rate for 473 intraluminal laser, 473 nm extraluminal laser, 473 nm subdiaphragmatic vagus, 473 nm cervical vagus, 532 nm intraluminal, and sucrose 300 mM+532 nm laser stimulation in CckCRE_ChR2-tdTomato mice (N≥3, error bars indicate S.E.M.). Stars indicate significant difference (p<0.05). Statistics by ANOVA and Tukey HSD post hoc analysis.

FIG. 15B shows vagal firing rate responded to sucrose (300 mM), sucrose (300 mM)+473 nm intraluminal laser, and sucrose (300 mM)+532 nm laser applied to the sub-diaphragmatic vagus in CckCRE_Halo-YFP mice. Normalized maximum firing rate for baseline, sucrose (300 mM), sucrose (300 mM)+532 nm intraluminal laser, 532 nm intraluminal laser, sucrose (300 mM)+473 nm intraluminal laser, 473 nm intraluminal laser, sucrose (300 mM)+532 nm extraluminal laser, and sucrose (300 mM)+532 nm laser applied to sub-diaphragmatic vagus in CckCRE_Halo-YFP mice (n≥3, error bars indicate S.E.M.). Stars indicate significant difference (p<0.05). Statistics by ANOVA and Tukey HSD post hoc analysis.

FIG. 15C shows real time qPCR expression of glutamate receptors in the nodose ganglia. The nodose ganglia expressed glutamate receptors in each of the four sub-types of receptors: AMPA, kinate, NMDA, and metabotropic. Values are reported as 2-ΔCt compared to 18S expression (mean±S.E.M.; n=3).

FIG. 16 shows vagal firing rate response to 300 mM sucrose, intraluminal, and extraluminal laser stimulation in Vglut1CRE_ChR2-YFP mice. Normalized maximum firing rate for 300 mM sucrose, and intraluminal, extraluminal, subdiaphragmatic vagus, and cervical vagus laser application in Vglut1CRE_ChR2-YFP mice (N≥3, error bars indicate S.E.M.). Stars indicate significant difference (p<0.05). Statistics by ANOVA and Tukey HSD post hoc analysis.

FIG. 17 shows body weights of PYYCRE_TLR5KO double transgenic mice (blue) were significantly more than the TLR5KO-only mice (red) as measured by a laboratory scale. Bars indicate the mean and the error bars indicate the standard error of the mean. Day 2 was the weight at the beginning of the two-day acclimation period. Day 0 marks the start of 3 consecutive days of weight measurement.

FIG. 18 shows body weights of PYYCRE_TLR5KO double transgenic mice and TLR5KO as measured continuously by Phenomaster and periodically by a laboratory scale. Box number is labeled above each graph (1, 3, 4, 5 had PYYCRE_TLR5KO; 2, 6, 7, 8 had TLR5KO-only). Black line indicates Phenomaster continuous measurements. Dots indicate laboratory scale measurements at 0, 24, 48, and 70 hours.

FIG. 19 shows food consumption of PYYCRE_TLR5KO (blue) mice and TLR5KO-only (red) mice during Diff1 (Day 0-1) and Diff2 (Day 1-2), and Diff3 (Day 2-3). Bars show the mean and error bars show the standard error of the mean.

FIG. 20 shows food consumption of PYYCRE_TLR5KO double transgenic mice and TLR5KO as measured continuously by Phenomaster and periodically by a laboratory scale. Boxes are labeled above each graph (1, 3, 4, 5 had PYYCRE_TLR5KO; 2, 6, 7, 8 had TLR5KO-only). Black lines indicate Phenomaster continuous measurements. Red Dots indicate laboratory scale measurements at 24, 48, and 70 hours. A mouse climbed into the feeder in Box 7, which accounts for the large drop in measured consumption.

FIG. 21 shows single cell real time qPCR by a heat map showing the expression of 88 genes in single enteroendocrine cells. Ct values are described 2\hat{ }-Ct, n=54 cells.

FIGS. 22A and 22B show altered expression of sensory receptors in MIA-enteroendocrine cells. These results are from a qRTPCR of 96 genes in a population of enteroendocrine cells. Compared to control (n=2) vs. MIA-autism model (n=3) male mice. MIA enteroendocrine cells had upregulated genes for several gut hormones: Peptide YY (Pyy), Glucagon (Gcg), Ghrelin (Ghrl), Cholecystokinin (Cck), and Neurotensin (Nts) (FIG. 22A); and the free fatty acid receptors Ffar1 and Ffar4, the bacterial receptor Nod1, and the irritant receptor Trpa1 (FIG. 22B).

FIG. 23 shows photostimulation of Cck enteroendocrine cells reduces food intake. CckCRE-Chr2_tdTomato mice are fitted with an abdominal window implant for the laser to penetrate into the intestinal lumen. Six days after surgery mice are fasted overnight. Then, mice are photostimulated using a 473 nm or 532 nm (control) laser for 30 min (40 Hz) at an intensity of 2 mW. Mice are then presented with food pellets. The number of pellets consumed is counted every 10 min. Food intake was monitored for the following 2 hours after stimulation. Photostimulation of enteroendocrine cells reduces food intake.

FIGS. 24A-24E show glutamate is used as a neurotransmitter between enteroendocrine cells and neurons. FIG. 24A shows a model of synaptic neurotransmission in enteroendocrine cells. FIG. 24B shows CckCRE_tdTomato enteroendocrine cells co-cultured with HEK cells that express the glutamate sniffer protein, iGluSnFR (multiphoton image-left; model-right). Scale bars=10 μm. FIG. 24C shows that stimulus of D-glucose-40 mM elicits a response in iGluSnFR-HEK cells (n=3). FIG. 24D shows co-culture with neurons and CckCRE_ChR2 cells (image-left) for electrophysiology of neurons and microperfusion of glutamate blocker, kynurenic acid (model-left). Scale bars=10 μm. FIG. 24E shows in co-culture, 473 nm photo-stimulation evoked EPSCs in connected nodose neurons, these currents were abolished with the addition of kynurenic acid-3 mM and recovered after the drug was washed off (n=4 pairs).

FIGS. 25A-25D show the rapid vagal response to sucrose is dependent on glutamate while CCK contributes to the prolonged response. FIG. 25A shows the normalized traces for baseline, sucrose-300 mM, sucrose-300 mM after devazepide-2 mg/kg, and sucrose-300 mM after glutamate inhibitor cocktail (KA/AP3=150 μg/kg kynurenic acid+1 mg/kg AP-3) in wild-type mice. FIG. 25B shows the normalized traces for baseline, sucrose-300 mM, and sucrose-300 mM after KA-150 μg/kg in wild-type mice. FIG. 25C shows KA/AP3 attenuates the maximum normalized vagal firing rate in response to sucrose while devazepide and KA alone do not. FIG. 25D shows KA/AP3 and KA along prolong time to peak from an average 92.8 seconds to 179 and 198 seconds respectively. Devazepide-2 mg/kg does not significantly change time to peak (mean=67.1 seconds). (n≥5 per group, shades and error bars=S.E.M).

FIGS. 26A-26B show that Fast Blue enema labels vagal neurons innervating the colon. FIG. 26A shows the left-control cervical vagus is left intact, and labeled cells are seen in the nodose ganglion (model-top, image-bottom). FIG. 26B shows the right-experimental cervical vagus is cut at the time of enema. No nodose neurons are labeled on the severed vagus side (n=3 mice) (top-model, bottom-image).

FIGS. 27A-27C show in vitro rabies transfection and retro-tracing in intestinal organoids co-cultured with vagal nodose neurons. FIG. 27A shows co-cultures of PGP9.5-GFP nodose neurons with CckCRE_tdTomato organoids shows that neurons grow out and make contact with CCK cells in organoids. Bars=10 μm. FIG. 27B shows wild-type nodose neurons are transduced by ΔG-rabies-mCherry before culturing with organoids. Wild-type neurons are not able to infect synaptically connected cells. Bars=10 μm. FIG. 27C shows Phox2bCRE_rabG-Tva neurons are transduced with EnvA-ΔG-rabies-mCherry before culturing with organoids. Rabies spreads mono-synaptically to transduce connected enteroendocrine cells. Bars=10 μm.

FIG. 28 shows that vagal firing rate responds rapidly to sucrose and 473 nm in CckCRE_ChR2-tdTomato and Vglut1CRE_ChR2-YFP mice. Time to peak and area under the curve (A.U.C.) for sucrose-300 mM and 473 nm intraluminal laser application in CckCRE_ChR2-tdTomato and Vglut1CRE_ChR2-YFP mice (n≥5, error bars indicate S.E.M.). No statistical significance. Statistics by ANOVA and Tukey HSD post hoc analysis.

FIGS. 29A-29B show that activation of VGlut1+ enteroendocrine cells is sufficient to drive a glutamatergic vagal response. FIG. 29A shows normalized traces for baseline, sucrose-300 mM, 473 nm intraluminal laser, and 473 nm intraluminal laser with glutamate inhibitor cocktail (KA/AP3=150 μg/kg kynurenic acid+1 mg/kg AP-3) in Vglut1CRE_Chr2-YFP mice.

FIG. 29B shows sucrose and 473 nm laser stimulate vagal activity in Vglut1CRE_ChR2. The response to laser is fully attenuated by KA/AP3.

FIGS. 30A-30D show that devazepide completely attenuates the vagal response to CCK. FIG. 30A shows normalized traces for baseline, CCK-870 nM (10 μg/kg), and CCK-870 nM following devazepide-2 mg/kg delivered intraluminally in wild-type mice. FIG. 30B shows normalized maximum firing rate for baseline before and after devazepide, CCK-870 nM before and after devazepide, and sucrose-300 mM before and after devazepide in wild-type mice. FIGS. 30C-30D show time to peak (FIG. 30C) and area under the curve (FIG. 30D) for CCK-870 nM after devazepide and sucrose-300 mM before and after devazepide (n≥5, error bars indicate S.E.M.). Statistics by ANOVA and Tukey HSD post hoc analysis.

FIG. 31 shows that devazepide and glutamate inhibitor cocktail reduce the magnitude of vagal response to sucrose in wild-type mice. Area under the curve (A.U.C.) was calculated for each 6-minute vagal cuff recording to represent magnitude of response. Devazepide-2 mg/kg and glutamate cocktail inhibitor (KA/AP3=150 μg/kg kynurenic acid (KA)+1 mg/kg AP-3) were delivered and sucrose response was assessed after delivery. Both devazepide and glutamate inhibitor reduced A.U.C. (n≥5, error bars indicate S.E.M.). Statistics by ANOVA and Tukey HSD post hoc analysis.

FIGS. 32A-32D show that kynurenic acid (KA), an ionotropic glutamate receptor inhibitor, attenuates rapid vagal response but not prolonged vagal response to sucrose. FIG. 32A shows normalized traces for baseline, sucrose-300 mM, and sucrose-300 mM after dose of KA: 1.5 μg/kg, 15 μg/kg, 150 μg/kg, and 1.5 mg/kg. FIG. 32B shows normalized maximum firing rate for baseline before and after KA, sucrose-300 mM prior to KA, and sucrose-300 mM after KA (doses above). FIGS. 32C-32D show time to peak (FIG. 32C) and area under the curve (FIG. 32D, A.U.C.) for sucrose-300 mM prior to KA and after KA (doses above). (n≥5, error bars indicate S.E.M.). Statistics by ANOVA and Tukey HSD post hoc analysis.

FIG. 33A-33B show that iGluSnFR-HEK cells respond to glutamate but not D-glucose. FIG. 33A shows HEK cells were transfected with the membrane bound iGluSnFR plasmid and cultured (model-left, image-right). FIG. 33B shows glutamate-100 μm but not Dglucose-40 mM causes an increase in fluorescence in iGluSnFR-HEK cells as measured by multiphoton microscopy (n=10).

FIGS. 34A-34C show that enteroendocrine cells transduce glucose stimuli onto vagal neurons. FIG. 34A shows model of intestinal intraluminal perfusion and vagal nerve electrophysiology. FIG. 34B shows normalized traces for baseline, sucrose-300 mM, and sucrose-300 mM+phloridzin-3 mM (phl) in wild-type mice. FIG. 34C shows Ensure®, sucrose-300 mM and D-glucose-150 mM stimulate vagal firing rate, which is abolished by SGLT1 blocker phloridzin (n≥5, shades and error bars=S.E.M)

### DETAILED DESCRIPTION

The present disclosure is based, in part, on the discovery of the components and functional connectivity of the gut neuroepithelial circuit; a neural circuit for fast transduction of gut-brain signals. It was discovered that enteroendocrine cells synapse with the vagus to transduce sensory signals from the gut to the brain. This gut-to-brain sensory neuroepithelial circuit can act as a portal for pathogens and a path for luminal stimuli, such as ingested sugars, to reach the brain. Enteroendocrine cells are constantly exposed to luminal contents of the small intestine and colon. Through the expression of cell membrane receptors, they recognize nutrients, bacterial, ligands, mechanical stretch, and perhaps thermal signals in the lumen of the small intestine and colon. The connection to neurons indicates that nerves can sense luminal contents through rapid neuronal excitation and pathogens that infect enteroendocrine cells can spread to the nervous system through direct synaptic links. A monosynaptic link between gut sensory cells, such as gut sensory epithelial cells, and nerves, such as vagal nodose neurons, was surprisingly discovered and observed both in vivo and in vitro. This neural circuit can constitute a path for pathogens in the gut to travel up to the brain, including viruses that cause neurodegenerative diseases. This claim is clearly shown by the rabies discovery and the ability to recapitulate the connection with organoids. Another discovery was that enteroendocrine cell, such as CCK enteroendocrine cells, produced signaling molecules that were previously ascribed only to enterochromaffin cells. The discovered neuroepithelial circuit was capable of transducing signals from nutrients, such as glucose, within milliseconds (60-800 ms), opening a physical path for fast sensory transduction from gut lumen to brain, analogous to that of taste transduction in the tongue. For example, the enteroendocrine cells transduced sensory signals from gut lumen, including that of glucose, within milliseconds to vagal nodose neurons.

A monosynaptic rabies virus and optogenetics was used to dissect details of this sensory transduction from the gut to the brain. To define if peripheral nerves connect with enteroendocrine cells, the monosynaptic rabies virus B19G SADΔG-GFP was used with a transgenic mouse model, PyyCRE_tdTomato rabG, that was developed to enable cell-specific spread of the virus. When delivered in the colon's lumen, there was visible GFP in mucosal nerves and nodose of PyyCRE_tdTomato rabG mice but not in controls. These data show that colonic enteroendocrine cells are innervated by vagal nerve fibers. To test neurotransmission in this neuroepithelial circuit in isolation, an in vitro co-culture system was developed using purified enteroendocrine cells and nodose ganglia neurons. The two cell types connected within 12-36 hrs and the connected cells often remained viable for at least 5 days, showing that this neuroepithelial circuit could be recapitulated in vitro. The possibility of afferent gut-to-brain transduction using whole cell electrophysiology was tested and it was discovered that a stimulus of 10 mM of glucose applied to the enteroendocrine cell induced excitatory post-synaptic potentials and action potential spikes in the connected neuron, while 10 mM glucose did not activate a nodose neuron by itself. These findings unveiled a gut-brain neuroepithelial circuit with the ability to transduce a chemical sense.

By synapsing with the vagus, these sensors provide a neuroepithelial circuit for fast sensory transduction. A gut sensory epithelial cells that synapse with nerves can be called a neuropod cell. The gut brain neural circuit can be formed by neuropod cells and nodose neurons The existence of this neural circuit leads to: 1) rapid distinction of stimuli based on physical (e.g. volume) versus chemical (e.g. calorie) composition; 2) precise topographical sensory representation of specific gastrointestinal regions; 3) localized plasticity encoded within the neural circuit, depending on the stimuli; 4) timely vagal efferent feedback to modulate gastrointestinal sensory function; and 5) given the conditions, a portal for gut-borne pathogens to gain access to the central nervous system. Enteroendocrine cells can use both, paracrine and neurotransmission signals, to help the brain make sense of what is being eaten by a subject.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Enterochromaffin cell” as used herein refers to a type of enteroendocrine and neuroendocrine cell that reside alongside the epithelium lining the lumen of the digestive tract. Enterochromaffin cells, also known as Kulchitsky cells, play a role in gastrointestinal regulation, particularly intestinal motility and secretion. Enterochromaffin cells modulate neuron signaling in the enteric nervous system (ENS) via the secretion of the neurotransmitter serotonin and other peptides. Enterochromaffin cells act as a form of sensory transduction as enteric afferent and efferent nerves do not protrude into the intestinal lumen.

“Enteroendocrine cells” as used herein refers to specialized cells of the gastrointestinal tract and pancreas with endocrine function. Enteroendocrine cells produce gastrointestinal hormones or peptides in response to various stimuli and release them into the bloodstream for systemic effect, diffuse them as local messengers, or transmit them to the enteric nervous system to activate nervous responses. Enteroendocrine cell constitute an enteric endocrine system as a subset of the endocrine system and are known to act as chemoreceptors, initiating digestive actions and detecting harmful substances and initiating protective responses. Enteroendocrine cells are located in the stomach, in the intestine and in the pancreas. Enteroendocrine cells can be intestinal enteroendocrine cells, gastric enteroendocrine cells, or pancreatic enteroendocrine cells, including but limited to, K cell, 1 cell, I cell, G cell, enterochromaffin cell, N cell, S cell, D cell, and M cell.

The terms “inhibitor”, “antagonist” or “blocker” refer to a compound or substance that decreases or blocks one or more activities of a protein of interest, for example, a sugar-sensing receptor, an amino acid-sensing receptor, a fatty acid-sensing receptor, or a bacteria-sensing receptor. In specific embodiments, terms “inhibitor”, “antagonist” or “blocker” in the context of the receptors, refer to a compound or substance that decreases the downstream signaling response associated with the receptor. In particular embodiments, decreasing receptor activity can result in change in the amount or distribution of an intracellular molecule or the activity of an enzyme which is part of the intracellular signaling pathway for the receptor. Examples of the intracellular molecule include, but are not limited to, free calcium, cyclic adenosine monophosphate (cAMP), inositol mono-, di- or tri-phosphate. Examples of the enzyme include, but are not limited to, adenylate cyclase, phospholipase-C, G-protein coupled receptor kinase.

“Subject” and “patient” as used herein interchangeably refers to any vertebrate, including, but not limited to, a mammal and a human. In some embodiments, the subject may be a human or a non-human. The subject or patient may be undergoing forms of treatment. “Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

“Treat,” “treating” or “treatment” are each used interchangeably herein to describe reversing, alleviating, or inhibiting the progress of a disease, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. METHODS OF MODULATING A TRANSSYNAPTIC SIGNAL THROUGH A NEUROEPITHELIAL CIRCUIT BETWEEN A GUT SENSORY CELL AND THE BRAIN

Embodiments of the present disclosure relate generally to a method of modulating a transsynaptic signal through a neuroepithelial circuit between a gut sensory cell and the brain. The method includes stimulating or inhibiting a receptor on the gut sensory cell, thereby stimulating or inhibiting the transsynaptic signal from the gut sensory cell to the brain. In some embodiments, stimulating or inhibiting the receptor on the gut sensory cell comprises contacting the gut sensory cell with a composition capable of stimulating or inhibiting the receptor on the gut sensory cell. In some embodiments, stimulating or inhibiting the receptor on the gut sensory cell comprises administering to the subject a therapeutically effective amount of a composition capable of stimulating or inhibiting the receptor on the gut sensory cell of the subject. In some embodiments, the composition includes a modulator of the receptor. In some embodiments, the receptor can be involved in sensing a carbohydrate, such as a sugar, a starch, or a cellulose, an amino acid, a protein, a fatty acid, a fat, or a bacteria. In some embodiments, the receptor can be a sugar-sensing receptor, an amino acid-sensing receptor, a fatty acid-sensing receptor, or a bacteria-sensing receptor.

A “sugar-sensing receptor” as used herein refers to receptor that binds and recognizes a sugar. In some embodiments, the sugar can be glucose. In some embodiments, the sugar-sensing receptor can be a sodium-dependent glucose cotransporters or sodium-glucose linked transporter (SGLT), such as SGFT1. SGFT1, which is a part of the family of glucose transporters, is found in the intestinal mucosa of the small intestine.

An “amino acid sensing receptor” as used herein refers to a receptor that binds and recognizes an amino acid. In some embodiments, the amino acid can be an L-amino acid, such as L-glutamate, or a D-amino acid. In some embodiments, the amino acid-sensing receptor can be a taste receptor type 1 member 1 (TAS1R1). TAS1R1, which is encoded by the TAS1R1 gene, is a G protein-coupled receptor with seven trans-membrane domains and is a component of the heterodimeric amino acid taste receptor T1R1+3. In some embodiments, the amino acid-sensing receptor can be a vesicular glutamate transporter, such as Vglut1.

A “fatty acid-sensing receptor” as used herein refers to a receptor that binds and recognizes a fatty acid. In some embodiments, the fatty acid can be a short chain saturated fatty acid, a short chain unsaturated fatty acid, a medium chain saturated fatty acid, a medium chain unsaturated fatty acid, a long chain saturated fatty acid, or a long chain unsaturated fatty acid. In some embodiments the fatty acid can be eicosatrienoic acid (20:3Δ11,14,17), linoleic acid, oleic acid or 10 carbons long. In some embodiments, the fatty acid-sensing receptor can be free fatty acid receptor 1 (FFA1), also known as GPR40, free fatty acid receptor 2 (FFA3), free fatty acid receptor 3 (FFA3), free fatty acid receptor 4 (FFA4, also known as G protein-coupled receptor 120 (GPR120)), or G protein-coupled receptor 119 (GPR119).

A “bacteria-sensing receptor” as used herein refers to a receptor that binds and recognizes structurally conserved molecules derived from microbes. In some embodiments, the molecules derived from microbes can be a bacterial lipoprotein, bacterial glycolipids, bacterial lipopolysaccharide, bacterial lipoteichoic acid, fungal zymosan (Beta-glucan), viral double-stranded RNA, poly I:C, bacterial heat shock proteins, peptidoglycan motifs from bacterial cell which consists of N-acetylglucosamine and N-acetylmuramic acid, such as meso-diaminopimelic acid (meso-DAP) or muramyl dipeptide (MDP). In some embodiments, the bacterial-sensing receptor can be Toll-like receptor 5 (TLR5), Toll-like receptor 1 (TLR1), Toll-like receptor 2 (TLR2), Toll-like receptor 3 (TLR3), Toll-like receptor 4 (TLR4), T1r1, T1r2, T1r3, T1r4, T1r5, nucleotide-binding oligomerization domain-containing protein 1 (NOD1), or nucleotide-binding oligomerization domain-containing protein 2 (NOD2).

In some embodiments, the receptor can be a sodium-dependent glucose cotransporter (SGLT), a taste receptor type receptor (TAS), a free fatty acid receptor (FFAR), a G-protein coupled receptor (GPR), a Toll-like receptor (TLR), a nucleotide-binding oligomerization domain-containing protein receptor (NOD), or a combination thereof. In some embodiments, the receptor can be SGLT1 or TLR5.

In some embodiments, the neuroepithelial circuit can include a nerve fiber. In some embodiments, the neuroepithelial circuit can include a gut sensory cell in contact with the nerve fiber. In some embodiments, the gut sensory cell is in contact or communication with the nerve fiber by releasing a neurotransmitter. In some embodiments, the neurotransmitter is glutamate. In some embodiments, the nerve fiber is a vagal nerve fiber or a sensory nerve fiber. In some embodiments, the vagal nerve fiber can include a vagal nodose neuron. In some embodiments, the neuroepithelial circuit can include a gut sensory cell in contact with dorsal root ganglia. In some embodiments, the gut sensory cell can include a gut epithelial cell. In some embodiments, the gut sensory cell can include an enteroendocrine cell and/or an enterochromaffin cell. By synapsing with vagal neurons, enteroendocrine cells can transduce signals up to the brain in a direct, selective, and temporally precise fashion. In some embodiments, the enteroendocrine cell is from the small intestine or colon. In some embodiments, the neuroepithelial circuit can include an enteroendocrine cell in contact with a vagal nerve fiber. In some embodiments, the neuroepithelial circuit can include an enteroendocrine cell in contact with a dorsal root ganglia neuron.

In some embodiments, the neuroepithelial circuit can be stimulated or inhibited by modulating the communication of the gut sensory cell with the nerve fiber. In some embodiments, modulating the communication of the gut sensory cell can include administering an inhibitor of the neurotransmitter. In some embodiments, the inhibitor of the neurotransmitter blocks the neurotransmitter from binding to the nerve fiber. In some embodiments, the neurotransmitter is glutamate and the inhibitor blocks an ionotropic glutamate receptor on the nerve fiber. In some embodiments, the ionotropic glutamate receptor is an N-methyl-D-aspartate (NMDA) receptor, an α-amino-3-hydroxy-5-methyl-4-isoxazolepropionic acid (AMPA) receptor or a kainate receptor. In some embodiments, the ionotropic glutamate receptor inhibitor is kynurenic acid or DL-2-amino-3-phosphonopropionic acid (AP-3). In some embodiments, the neurotransmitter is cholecystokinin (CCK) and the inhibitor blocks CCK-A receptors. In some embodiments the CCK-A receptor is inhibited with devazepide.

a. Modulators of the Receptor

In some embodiments, stimulating or inhibiting a receptor on the gut sensory cell can include contacting the gut sensory cell with a composition capable of stimulating or inhibiting the receptor on the gut sensory cell. In some embodiments, stimulating or inhibiting a receptor on the gut sensory cell can include administering to the subject a therapeutically effective amount of a composition capable of stimulating or inhibiting the receptor on the gut sensory cell of the subject. In some embodiments, the composition can include a modulator of the receptor.

In some embodiments, the modulator of the receptor can include an agonist of the receptor, an antagonist of the receptor, and/or an inhibitor of the receptor.

In some embodiments, the modulation is achieved using a modulator of the sugar-sensing receptor. In some embodiments, the modulator can be an agonist, antagonist or inhibitor of the sugar-sensing receptor. For example, the modulator can be an SGLT1 inhibitor, such as GSK1614235, phloridzin, such as phloridzin dihydrate, sotagliflozin, DSP-3235, or T-1095. In some embodiments, the modulator of the receptor can include an SGLT1 inhibitor. In some embodiments, the SGLT1 inhibitor can be phloridzin dehydrate.

In some embodiments, the modulation is achieved using a modulator of the amino acid-sensing receptor. In some embodiments, the modulator can be an agonist, antagonist or inhibitor of the amino acid-sensing receptor. For example, the modulator can be a TAS1R1 inhibitor, such as lactisole.

In some embodiments, the modulation is achieved using a modulator of the fatty acid-sensing receptor. In some embodiments, the modulator can be an agonist, antagonist or inhibitor of the fatty acid-sensing receptor. For example, the modulator can be a FFA1 antagonist, such as DC 260126, a FFA1 inhibitor, such as GW-1100 (ethyl 4-[5-[(2-ethoxypyrimidin-5-yl)methyl]-2-[(4-fluorophenyl)methylsulfanyl]-4-oxopyrimidin-1-yl]benzoate), a FFA2 antagonist, such as GLPG 0974, or a FFA4 inhibitor, such as AH 7614.

In some embodiments, the modulation is achieved using a modulator of the bacteria-sensing receptor. In some embodiments, the modulator can be an agonist, antagonist or inhibitor of the bacteria-sensing receptor. For example, the modulator can be a TLR4 Antagonist, such as Lipopolysaccharide from R. sphaeroides (LPS-RS).

In some embodiments, the composition can include a sugar, an amino acid, a fatty acid, a bacteria, or a molecule derived from a bacteria. In some embodiments, the composition can include a non-metabolizable sugar, such as alpha-methyl-D-glucopyranoside (αMG). In some embodiments, the sugar can include glucose, sucrose, fructose, sucralose, or a combination thereof.

In some embodiments, the composition can include between about 10 mM and about 300 mM of sugar. In some embodiments, the composition can include between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of sugar. In some embodiments, the composition can include at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 250 mM, or at least about 300 mM of sugar. In some embodiments, the composition can include at least about 100 mM sugar.

In some embodiments, the composition can include between about 10 mM and about 300 mM of glucose. In some embodiments, the composition can include between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of glucose. In some embodiments, the composition can include at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 250 mM, or at least about 300 mM of glucose. In some embodiments, the composition can include at least about 300 mM glucose.

In some embodiments, the composition can include between about 10 mM and about 300 mM of sucrose. In some embodiments, the composition can include between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of sucrose. In some embodiments, the composition can include at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 250 mM, or at least about 300 mM of sucrose. In some embodiments, the composition can include at least about 300 mM sucrose.

In some embodiments, the composition can include between about 10 mM and about 300 mM of sucralose. In some embodiments, the composition can include between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of sucralose. In some embodiments, the composition can include at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 250 mM, or at least about 300 mM of sucralose. In some embodiments, the composition can include at least about 15 mM of sucralose.

In some embodiments, the composition can include between about 10 mM and about 500 mM of fructose. In some embodiments, the composition can include between about 10 mM and about 500 mM, between about 10 mM and about 400 mM, between about 10 mM and about 300 mM, between about 10 mM and about 250 mM, between about 10 mM and about 200 mM, between about 10 mM and about 150 mM, between about 10 mM and about 100 mM, between about 10 mM and about 50 mM, between about 15 mM and about 300 mM, between about 15 mM and about 250 mM, between about 15 mM and about 200 mM, between about 15 mM and about 150 mM, between about 15 mM and about 100 mM, between about 15 mM and about 50 mM, between about 50 mM and about 300 mM, between about 50 mM and about 250 mM, between about 50 mM and about 200 mM, between about 50 mM and about 150 mM, between about 50 mM and about 100 mM, between about 100 mM and about 300 mM, between about 100 mM and about 250 mM, between about 100 mM and about 200 mM, between about 100 mM and about 150 mM, between about 150 mM and about 300 mM, between about 150 mM and about 250 mM, or between about 150 mM and about 200 mM of fructose. In some embodiments, the composition can include at least about 10 mM, at least about 15 mM, at least about 20 mM, at least about 25 mM, at least about 30 mM, at least about 35 mM, at least about 40 mM, at least about 45 mM, at least about 50 mM, at least about 75 mM, at least about 100 mM, at least about 125 mM, at least about 150 mM, at least about 175 mM, at least about 200 mM, at least about 250 mM, at least about 300 mM, at least about 400 mM, or at least about 500 mM of fructose. In some embodiments, the composition can include at least about 150 mM of fructose.

In some embodiments, the composition can include a probiotic or a mixture of probiotics. A probiotic treatment can be administered through food or a pill to be an effective and useful treatment for patients with autism spectrum disorder, as discussed below. Designer probiotic can be designed to manipulate enteroendocrine cell function to correct behavior abnormalities. The designer probiotic can be a commensal gut bacteria that has been genetically engineered to produce a ligand that stimulates or inhibits enteroendocrine cell function.

### 3. METHODS OF ALTERING NUTRIENT SENSING IN A SUBJECT

The present disclosure also relates to methods of altering nutrient sensing in a subject by modulating a transsynaptic signal through a neuroepithelial circuit between a gut sensory cell and the brain. The method includes stimulating or inhibiting a receptor on the gut sensory cell, thereby stimulating or inhibiting the transsynaptic signal from the gut sensory cell to the brain and altering the nutrient sensing in the subject. By these the methods, the subject senses the nutrient in a manner that is different than if the receptor was not stimulated or inhibited in the subject. In some embodiments, stimulating the transsynaptic signal from the gut sensory cell to the brain increases the sensitivity of the subject to the nutrient. In some embodiments, stimulating the transsynaptic signal from the gut sensory cell to the brain decreases the sensitivity of the subject to the nutrient. In some embodiments, the nutrient can be a carbohydrate, such as a sugar, a starch, or a cellulose, an amino acid, a protein, a fatty acid, or a fat.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described above. In some embodiments, the composition includes a modulator of the receptor, as described above. In some embodiments, the receptor can be involved in sensing a sugar, an amino acid, a fatty acid, or a bacteria, as described above. In some embodiments, the receptor can be a sugar-sensing receptor, an amino acid-sensing receptor, a fatty acid-sensing receptor, or a bacteria-sensing receptor, as described above.

a. Methods of Modulating a Caloric Value of a Nutrient to a Subject

The present disclosure also provides methods of modulating a caloric value of a nutrient to a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain and modulating the caloric value of the nutrient to the subject. In some embodiments, the caloric value of the nutrient to the subject is increased. In some embodiments, the caloric value of the nutrient to the subject is decreased. In some embodiments, the nutrient can be a carbohydrate, such as a sugar, a starch, or a cellulose, an amino acid, a protein, a fatty acid, or a fat.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described above. In some embodiments, the composition includes a modulator of the receptor, as described above. In some embodiments, the receptor can be involved in sensing a sugar, an amino acid, a fatty acid, or a bacteria, as described above. In some embodiments, the receptor can be a sugar-sensing receptor, an amino acid-sensing receptor, a fatty acid-sensing receptor, or a bacteria-sensing receptor, as described above.

### 4. METHODS OF MODULATING A BACTERIAL STIMULUS SIGNAL IN A SUBJECT

The present disclosure also provides methods of modulating a bacterial stimulus signal in a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain and modulating the bacterial stimulus signal in the subject. In some embodiments, the bacterial stimulus signal in the subject is increased. In some embodiments, the bacterial stimulus signal in the subject is decreased.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described above. In some embodiments, the composition includes a modulator of the receptor, as described above. In some embodiments, the receptor can be involved in sensing a sugar, an amino acid, a fatty acid, or a bacteria, as described above. In some embodiments, the receptor can be a sugar-sensing receptor, an amino acid-sensing receptor, a fatty acid-sensing receptor, or a bacteria-sensing receptor, as described above.

### 5. METHODS OF ALTERING EATING BEHAVIOR OF A SUBJECT

The present disclosure also relates to methods of altering behavior of a subject by modulating a transsynaptic signal through a neuroepithelial circuit between a gut sensory cell and the brain. The method includes stimulating or inhibiting a receptor on the gut sensory cell, thereby stimulating or inhibiting the transsynaptic signal from the gut sensory cell to the brain and altering the nutrient sensing in the subject. By these the methods, the subject alters the eating behavior of the subject, such as altering appetite, food intake, and/or food preference, in a manner that is different than if the receptor was not stimulated or inhibited in the subject. In some embodiments, stimulating the transsynaptic signal from the gut sensory cell to the brain increases the appetite of the subject. In some embodiments, stimulating the transsynaptic signal from the gut sensory cell to the brain decreases the appetite of the subject.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described above. In some embodiments, the composition includes a modulator of the receptor, as described above. In some embodiments, the receptor can be involved in sensing a sugar, an amino acid, a fatty acid, or a bacteria, as described above. In some embodiments, the receptor can be a sugar-sensing receptor, an amino acid-sensing receptor, a fatty acid-sensing receptor, or a bacteria-sensing receptor, as described above.

a. Methods of Modulating Food Intake Behavior in a Subject

The present disclosure also provides methods of modulating food intake behavior in a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the food intake in a subject is modulated. In some embodiments, the food intake behavior in the subject is increased. In some embodiments, the food intake behavior in the subject is decreased.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described above. In some embodiments, the composition includes a modulator of the receptor, as described above. In some embodiments, the receptor can be involved in sensing a sugar, an amino acid, a fatty acid, or a bacteria, as described above. In some embodiments, the receptor can be a sugar-sensing receptor, an amino acid-sensing receptor, a fatty acid-sensing receptor, or a bacteria-sensing receptor, as described above.

b. Methods of Modulating Food Preference in a Subject

The present disclosure also provides methods of modulating food preference in a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the food preference behavior in the subject is modulated. In some embodiments, the food intake behavior and/or food preference in the subject is increased. In some embodiments, the food intake behavior and/or food preference in the subject is decreased.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described above. In some embodiments, the composition includes a modulator of the receptor, as described above. In some embodiments, the receptor can be involved in sensing a sugar, an amino acid, a fatty acid, or a bacteria, as described above. In some embodiments, the receptor can be a sugar-sensing receptor, an amino acid-sensing receptor, a fatty acid-sensing receptor, or a bacteria-sensing receptor, as described above.

### 6. METHODS OF TREATING A SUBJECT HAVING OR SUSPECTED OF HAVING AN EATING DISORDER

The present disclosure also provides methods of treating a subject having or suspected of having an eating disorder. The method involves modulating brain function through the GI tract to treat eating disorders that have both neurological and gastrointestinal components, such as anorexia and bulimia. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein eating behavior of the subject is modulated and the subject is treated. In some embodiments, the eating behavior of the subject is increased. In some embodiments, the eating behavior of the subject is decreased.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described above. In some embodiments, the composition includes a modulator of the receptor, as described above. In some embodiments, the receptor can be involved in sensing a sugar, an amino acid, a fatty acid, or a bacteria, as described above. In some embodiments, the receptor can be a sugar-sensing receptor, an amino acid-sensing receptor, a fatty acid-sensing receptor, or a bacteria-sensing receptor, as described above.

### 7. METHODS OF MODULATING ANXIETY IN A SUBJECT

The present disclosure also relates to methods of modulating anxiety in a subject. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the anxiety of the subject is modulated. In some embodiments, the anxiety of the subject is increased. In some embodiments, the anxiety of the subject is decreased.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described above. In some embodiments, the composition includes a modulator of the receptor, as described above. In some embodiments, the receptor can be involved in sensing a sugar, an amino acid, a fatty acid, or a bacteria, as described above. In some embodiments, the receptor can be a sugar-sensing receptor, an amino acid-sensing receptor, a fatty acid-sensing receptor, or a bacteria-sensing receptor, as described above.

### 8. METHOD OF TREATING AUTISM SPECTRUM DISORDERS BY MODULATING GUT SENSING IN A SUBJECT

The present disclosure also relates to methods of treating autism spectrum disorders (ASD) in a subject. The methods act on the gut-brain axis, and aims to treat the neurological symptoms of autism by treating the GI disorders among ASD patients. The method includes modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the eating behavior of the subject is modulated and the subject is treated for autism. In some embodiments, the eating behavior of the subject is increased. In some embodiments, the eating behavior of the subject is decreased.

In some embodiments, stimulating or inhibiting the receptor on the gut sensory cell comprises contacting the gut sensory cell with a composition capable of stimulating or inhibiting the receptor on the gut sensory cell, as described. In some embodiments, stimulating or inhibiting the receptor on the gut sensory cell comprises administering to the subject a therapeutically effective amount of a composition capable of stimulating or inhibiting the receptor on the gut sensory cell of the subject, as described above. In some embodiments, the composition includes a modulator of the receptor, as described above. In some embodiments, the receptor can be involved in sensing a sugar, an amino acid, a fatty acid, or a bacteria, as described above. In some embodiments, the receptor can be a sugar-sensing receptor, an amino acid-sensing receptor, a fatty acid-sensing receptor, or a bacteria-sensing receptor, as described above. In some embodiments, a probiotic bacteria, or combination of probiotic bacteria, can be used to directly target sensory enteroendocrine cells (cells within the gut that communicate with the brain) to modulate neurological behaviors.

### 9. COMPOSITIONS, PHARMACEUTICAL COMPOSITIONS, AND FORMULATIONS

Embodiments of the present disclosure also provide compositions, pharmaceutical compositions, and formulations that include at least one modulator of the receptor. The disclosed compositions, pharmaceutical compositions, and formulations can be used to treat or alleviate the symptoms of subjects that are diagnosed with or determined as having an eating disorder or ASD. The disclosed compositions, pharmaceutical compositions, and formulations can include at least one modulator of the receptor.

The compositions, pharmaceutical compositions, and formulations may include a “therapeutically effective amount” or a “prophylactically effective amount” of the modulator of the receptor. A “therapeutically effective amount” refers to an amount effective, at dosages and for periods of time necessary, to achieve the desired therapeutic result. A therapeutically effective amount of the compositions may be determined by a person skilled in the art and may vary according to factors such as the disease state, age, sex, and weight of the individual, and the ability of the compositions to elicit a desired response in the individual. A therapeutically effective amount is also one in which any toxic or detrimental effects of the modulator of the receptor, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of the modulator of the receptor, calculated to produce the desired therapeutic effect in association with the required pharmaceutical carrier. The specification for the dosage unit forms are dictated by and directly dependent on (a) the unique characteristics of modulator of the receptor, and the particular therapeutic or prophylactic effect to be achieved, and (b) the limitations inherent in the art of compounding such composition that modulates the receptor.

It is to be noted that dosage values may vary with the type and severity of the condition to be alleviated. Further, the modulator of the receptor dose may be determined by a person skilled in the art and may vary according to factors such as the disease state, age, sex, and weight of the individual, and the ability of the modulator of the receptor to elicit a desired response in the individual. The dose is also one in which toxic or detrimental effects, if any, of the modulator of the receptor are outweighed by the therapeutically beneficial effects. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The compositions, pharmaceutical compositions, and formulations may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Various delivery systems are known and can be used to administer one or more of modulator of the receptor, and a prophylactic agent or therapeutic agent useful for preventing, managing, treating, or ameliorating the eating disorder, such as bulimia or anorexia, or one or more symptoms thereof, e.g., encapsulation in liposomes, microparticles, microcapsules. Methods of administering a prophylactic or therapeutic agent of the invention include, but are not limited to, parenteral administration (e.g., intradermal, intramuscular, intraperitoneal, intravenous and subcutaneous), epidurala administration, intratumoral administration, and mucosal administration (e.g., intranasal and oral routes). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

If the pharmaceutical composition is administered orally, the pharmaceutical compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The pharmaceutical compositions may be administered by and formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use. The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The pharmaceutical compositions may be formulated as neutral or salt forms. Pharmaceutically acceptable salts include those formed with anions such as those derived from hydrochloric, phosphoric, acetic, oxalic, tartaric acid, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, compositions can be dispensed with an infusion bottle containing sterile pharmaceutical grade water or saline. Where the mode of administration is by injection, an ampoule of sterile water for injection or saline can be provided so that the ingredients may be mixed prior to administration.

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application.

In certain embodiments, modulator of the receptor, may be orally administered, for example, with an inert diluent or an assimilable edible carrier. The modulator of the receptor (and other ingredients, if desired) may also be enclosed in a hard or soft shell gelatin capsule, compressed into tablets, or incorporated directly into the subject's diet. For oral therapeutic administration, the modulator of the receptor may be incorporated with excipients and used in the form of ingestible tablets, buccal tablets, troches, capsules, elixirs, suspensions, syrups, wafers, and the like. To administer the modulator of the receptor by other than parenteral administration, it may be necessary to coat the modulator of the receptor with, or co-administer the modulator of the receptor with, a material to prevent its inactivation.

Modulator of the receptor, can be used alone or in combination to treat the eating disorder or ASD. It should further be understood that the combinations are those combinations useful for their intended purpose.

### 10. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

### Materials and Methods

Animals.

Mouse care and experiments were carried out in accordance with protocols approved by the Institutional Animal Care and Use Committee at Duke University Medical Center under the protocol A009-16-01. Mice were housed in the Duke University animal facilities, where they were kept on a 12-hour light dark cycle. They received food and water ad libitum. The following strains were used: C57B16 (4-12 weeks), Cck-GFP (4-12 weeks), Pgp9.5-GFP (4-12 weeks) (Table 1).

TABLE 1
Mouse Strain Information

| Mouse strain | Source | Stock Number |
|---|---|---|
| C57B16 | Jackson Lab | 000664 |
| Cck-GFP | Courtesy of | (Wang et al. (2010) American |
| | Rodger Liddle | Journal of Physiology- |
| | | Gastrointestinal and Liver |
| | | Physiology 300: G528-G537 |
| Pgp9.5-GFP | Jackson Lab | 024355 |
| CckCRE | Jackson Lab | 012706 |
| PyyCRE | Courtesy of | (Schonhoff et al. (2005) |
| | Andrew Leiter | Molecular and Cellular |
| | | Biology 25: 4189-4199) |
| Phox2bCRE | Jackson Lab | 016223 |
| VglutCRE | Jackson Lab | 023527 |
| LSL_tdTomato | Jackson Lab | 007914 |
| LSL_ChR2- | Jackson Lab | 012567 |
| tdTomato | | |
| LSL_ChR2-YFP | Jackson Lab | 007612 |
| LSL_RabG-Tva | Jackson Lab | 024708 |
| LSL_GCaMP6s | Jackson Lab | 024106 |
| LSL_Halo-YFP | Jackson Lab | 014539 |

*LSL = loxP-STOP-loxP cassette

Double transgenic mice were all bred in house: CckCRE_tdTomato (6-12 weeks), CckCRE_ChR2-tdTomato (6-12 weeks), PyyCRE_RabG-Tva (P8), Phox2bCRE_GCaMP6s (6-12 weeks), CckCRE_RabG-Tva (P8), PyyCRE_tdTomato (6-12 weeks), CckCRE_Halo-YFP (6-12 weeks), and Vglut1CRE_ChR2-YFP (6-12 weeks).

Rabies Production.

G-deleted rabies virus production was performed in house as previously described (Wickersham et al., Monosynaptic restriction of transsynaptic tracing from single, genetically targeted neurons. Neuron 53, 639-647 (2007)). For rescue from cDNA, B19G cells were plated on 10 cm dishes in DMEM with 10% FBS, Glutamax, and NEAA on the day before transfection. At 85% confluence, the cells were transfected using Lipofectamine2000 (Invitrogen) with genome vector 38 μg cSPBN-4GFP (cat. #52487, Addgene) or 38 μg pRVdG-4mCherry (cat. #52488, Addgene), 18 μg pCAG B19N (cat. #59924, Addgene), 9 μg pCAG B19P (cat. #59925, Addgene), 7.5 μg pCAG B19G (cat. #59921, Addgene), 9 μg pCAG B19L (cat. #59922, Addgene), and 11 μg pCAG T7 pol (cat. #59926, Addgene). The following day the media was changed to DMEM with 2% FBS, GM, NEAA and Sodium pyruvate. Viral supernatants were collected every three days while the cells remained healthy. Supernatants were filtered with a 0.45 μm filter, and concentrated by ultracentrifugation at 26K-rpm through a 20% sucrose cushion. If necessary, viral stocks were further amplified on B19G cells. EnvA pseudotyped virus was prepared by infecting BHK-EnvARGCD2 cells with G-deleted B19G rabies virus. The next day, the cells were rinsed twice with PBS, exposed to 0.25% trypsin for 5 minutes and re-plated on new 10 cm dishes. One day later the cells were again rinsed twice with PBS. Supernatant was collected every three days, 0.45 um filtered, and concentrated by ultracentrifugation.

Organoid Culture.

The small intestines of CckGFP or wild-type mice were dissected and flushed twice with cold PBS. The intestine was cut open lengthwise and cut again into ˜1 cm pieces. Tissue pieces were incubated with 2.5 mM EDTA at 4° C. for 15 minutes then at 37° C. for 15 minutes. New cold PBS was added and crypts were mechanically detached. Crypts were spun down and re-suspended in Matrigel (Corning #356231). Crypts in Matrigel were plated 50 μl per well in a 24-well plate. After 15 minutes incubation at 37° C., 500 μl of media were added to each well. Organoid media contains 10 μl/ml Glutamax, 10 μl/ml HEPES, 20 μl/ml Penicillin-Streptomycin, 10 μl/ml N2 supplement, 20 μl/ml B27 supplement, 0.25 μl/ml EGF, 0.5 μl/ml Noggin, and 1 μl/ml r-Spondin in Advanced DMEM/f12.

Rabies Tracing.

For colon monosynaptic tracing, P1 mice were given a 10 μl enema of EnvARv-ΔG-GFP (5.9×109 ffu/ml). The experimental mice used were PyyCRE_RabG-Tva (n=9), with negative genotype littermates as controls (n=4). One control mouse was not included in the data set due to extensive colon damage caused by the enema. For small intestine monosynaptic tracing, P1 mice were given a 50 μl gavage of Rv-ΔG-GFP (9.8×108 ffu/ml). The experimental mice used were CckCRE_RabG-Tva (n=3), with negative genotype littermates as controls (n=2). Mice were sacrificed 7 days after exposure at P8. Tissue harvested included the colon (or stomach and small intestine), lumbar/sacral DRGs, nodose ganglia, and brain. Tissue was post-fixed in 4% PFA for three hours and then treated with serial sucrose solutions of 10%, 20%, and 30%. Ganglia were whole-mount imaged with a multi photon microscopy system (Bruker Ultima IV with a Chameleon Vision II tunable laser). All other tissue was frozen in OCT blocks and sectioned for immunohistochemistry.

Enteroendocrine Cell and Nodose Neuron Co-Culture.

The small intestines of CckGFP and CckCRE_ChR2-tdTomato mice were dissected and flushed twice with cold PBS. The intestine was cut open lengthwise and cut again into ˜1 cm pieces. Tissue pieces were incubated with 3 mM EDTA at 4° C. for 15 minutes then at 37° C. for 15 minutes. New cold PBS was added and villi and crypts were mechanically detached. Villi and crypts were spun down and further digested in Collagenase and Dispase. Cells were spun down and re-suspended in L-15 media with 5% FBS and DNase to prevent cell clumping. Cells were passed through a 70 μm then 40 μm cell strainer to isolate single cells. Then cells were sorted using Fluorescence Activated Cell sorting (BD FACSAria) selecting for GFP fluorescence cells. Cells were sorted into culture media: 10 μl/ml Glutamax, 10 μl/ml HEPES, 20 μl/ml Penicillin-Streptomycin, 10 μl/ml N2 supplement, 20 μl/ml B27 supplement, 0.25 μl/ml EGF, 0.5 μl/ml Noggin, 1 μl/ml r-Spondin and 1 μl/ml NGF in Advanced DMEM/f12. Sorted cells were plated on Matrigel coated 12 mm coverslips at a concentration of ˜8-10 k enteroendocrine cells per coverslip. Nodose neurons were dissected and incubated with Liberase digestion enzyme. Enzyme was washed off and replaced with media. Single cells were dissociated by mechanical force and filtered through a 70 μm cell strainer. Neurons in media were plated evenly on up to 8 coverslips with enteroendocrine cells. Patch-clamp electrophysiology was performed 1-5 days after plating.

Immunohistochemistry.

Tissue sections were cut at a thickness of 10-16 μm, then post-fixed in 10% NBF for 10 minutes and washed in TBS+0.05% Tween20 (TBST). Sections were blocked with 10% Donkey Serum for 1 hour prior to being incubated overnight at 4° C. with primary antibody [Rb-Anti-PYY [DVB3] (1:1000); Rb-Anti-CCK (1:1000; courtesy of Rodger Liddle or Phoenix Pharmaceuticals H-069-04); Gt-Anti-PSD95 (1:500; Santa Cruz Biotechnology: sc-6926); Rb-Anti-Syn1 (1:500; Cell Signaling Technology: 5297S); Ck-Anti-GFP (1:500; Abcam:ab13970]. After TBST washes, sections were incubated with secondary antibody (Jackson ImmunoResearch) for one hour at room temperature [Dk-Anti-Rb-488 (1:250); Dk-Anti-Rb-Cy3 (1:250); Dk-Anti-Gt-Cy5 (1:250); Dk-Anti-Ck-488 (1:250)]. Slides were washed with TBST, then incubated with DAPI and mounted for imaging. Imaging was done on a Zeiss 880 Airyscan inverted confocal microscope. Data are presented as the mean percentage±S.E.M.

Real-Time Quantitative PCR.

Cck-GFP small intestine was dissociated and cell sorted as described above. An equal number of GFP+ and GFP− cells were collected directly into lysis buffer. Nodose was dissected and flash frozen in liquid nitrogen. For all tissue, RNA was extracted based on the manufacturer's protocol using the RNeasy Micro Plus Kit (Qiagen #74034). Then cDNA was produced per the manufacturer's protocol using the High Capacity cDNA Reverse Transcription Kit (Applied Biosystems #:4368814). TaqMan probes were used for transcript identification (Table 2). Real-time qPCR was run on a StepOnePlus System (Thermo Fischer), using TaqMan Fast Universal PCR Master Mix (Applied Biosystems #: 4352042) according to the manufacturer's protocol. Transcription rate was determined as 2-ΔCt, or compared as fold-change over EEC negative epithelial cells using 2-ΔΔCt. All values are reported as mean±S.E.M.

TABLE 2
TaqMan probes used for transcript identification
in Real-time quantitative PCR.

| Gene | TaqMan ID |
|---|---|
| 18S | Mm03928990_g1 |
| Cask | Mm00438021_m1 |
| Cck | Mm00446170_m1 |
| Efnb2 | Mm00438670_m1 |
| Gria1 | Mm00433753_m1 |
| Gria2 | Mm00442822_m1 |
| Gria3 | Mm00497506_m1 |
| Grik3 | Mm01179716_m1 |
| Grik5 | Mm00433774_m1 |
| Grin1 | Mm00433790_m1 |
| Grm7 | Mm01189424_m1 |
| Grm8 | Mm00433840_m1 |
| Lrrc4 | Mm11166846_s1 |
| Lrrtm2 | Mm00731288_s1 |
| Nrxn1 | Mm00660298_m1 |
| Nrxn2 | Mm01236851_m1 |
| Nrxn3 | Mm04279482_m1 |
| Pyy | Mm00520716_g1 |
| Reg4 | Mm00471115_m1 |
| Sct | Mm01275684_g1 |
| Slc17a6 | Mm00499876_m1 |
| Slc17a7 | Mm00812886_m1 |
| Slc5a1 | Mm00451203_m1 |
| Slc5a2 | Mm00453831_m1 |
| Syn1 | Mm00449772_m1 |

Electrophysiology.

Enteroendocrine cells and nodose neurons were co-cultured as described above. Co-culture coverslips were placed in the recording chamber filled with extracellular solution. Recordings were carried out at room temperature using a MultiClamp 700B amplifier (Axon Instruments), digitized using a Digidata 1550A (Axon Instruments) interface, and pClamp software (Axon Instruments) for data acquisition. Data was filtered at 1 kHz and sampled at 10 kHz. CCK-producing enteroendocrine cells were identified by GFP expression and neurons by their morphology and lack of fluorescence. Recordings were made using borosilicate glass pipettes pulled to ˜3.5 MΩ resistance [DVB4]. Extracellular solution contained (in mM): 140 NaCl, 5 KCl, 2 CaCl2, 2 MgCl2, 10 HEPES, pH 7.4 (300-305 mOsmol). For voltage-clamp recordings, intracellular solution contained (in mM): 140 CsF, 10 NaCl, 0.1 CaCl2, 2 MgCl2, 1.1 EGTA, 10 HEPES, 10 sucrose (pH, 7.25, 290-295 mOsmol). For current-clamp recordings, intracellular solution contained (in mM): 140 KCl, 0.5 EGTA, 5 HEPES, 3 Mg-ATP, 10 sucrose (pH, 7.25, 290-295 mOsmol). Neurons were held at −70 mV for 5 min after patching in voltage-clamp mode (to stabilize cells), and then switched to the current-clamp mode. At the resting membrane potential, cell properties such as input resistance, and spike threshold were determined using 200 ms steps from −40 pA to 100 pA in 20 pA increment. After that, baseline neuron activity was recorded in current-clamp or voltage-clamp for 5 min before exposure to glucose (20 mM) for 30-60 sec, followed by a wash with no glucose-containing extracellular solution. After glucose exposure, neurons were tested with a current or voltage pulse to confirm the health of the cell. For glutamate receptor blocker experiments, the SMARTSQUIRT® Micro-perfusion system (Automate Scientific, Inc.) was used. Once the neuron was patched, the SMARTSQUIRT® nozzle was brought to within 100 μm of the cell, while extracellular solution was perfusing. Kynurenic acid (3 mM) was perfused, then washed for 5 minutes after and the neuron was tested again. EPSC responses were recorded for 1 second after light stimulation. Data are presented as the mean±S.E.M. and significance was determined using a two-tailed Student's t test.

Vagus Nerve Recording.

Wild-type control (n=5-9), CckCRE_ChR2-tdTomato (n=6), CckCRE_Halorhodopsin-YFP (n=5), and Vglut1CRE_ChR2-YFP (n=6) mice were used for vagal recordings. Mice were anesthetized with 3 mg/g-mouse urethane 1 hour before surgery then with 0.5-1% isoflurane. A section of neck skin was removed between the jaw and clavicle. The thyroid was reflected bilaterally, and the carotid bundle was exposed using coarse dissection. The carotid nerve was separated from the vagus nerve using a curved blunt probe. Two platinum iridium wires (0.001″ diameter, PTFE coated, 10% Ir 90% Pt; Medwire by Sigmund Cohn Corp) were looped around the vagus nerve for recording. Mineral oil was used to insulate the vagus nerve from surrounding tissue. The small intestine was exposed by removing a section of skin and peritoneum. A 20 gauge gavage needle was surgically inserted through the stomach wall and into the duodenum. The gavage needle was sutured in place beyond the pyloric sphincter. Saline and stimulant tubes were connected to the gavage needle. For optogenetic experiments, a fiber optic cable (FT020, ThorLabs) was threaded through the gavage needle into the lumen of the duodenum. A perfusion exit incision was made 10 cm distal to the pyloric sphincter. During each recording, PBS was constantly perfused through the duodenum using a peristaltic pump (Cole-Parmer) at the lowest setting for a flow rate of ˜400 uL PBS per minute. Stimulation conditions were applied after recording 2 minutes of baseline activity. During whole nutrient stimulation conditions, PBS perfusion was continuous and 200 μL of ENSURE® was perfused through the second tube for 1 minute using a syringe pump. During distinctive nutrient stimulation conditions, PBS perfusion was continuous, and 200 μL of stimulant (1M sucrose, 500 mM fructose, 500 mM D-glucose, or 1M sucrose with 8.47 mM (0.4% w/v) phloridzin) was perfused through the second tube for 1 minute using a syringe pump (Fusion 200, Chemyx) for a final concentration of ˜300 mM sucrose in PBS (600-700 mOsm), ˜150 mM D-glucose in PBS (˜500 mOsm), and fructose ˜150 mM in PBS (˜500 mOsm). During CCK stimulation conditions, PBS perfusion was continuous, and 200 μL of 2.9 μM CCK octapeptide-sulfated (BaChem) was perfused through the second tube for 1 minute using a syringe pump (Fusion 200, Chemyx) for a final concentration of ˜870 nM CCK in PBS (10 μg/kg dose). For experiments using the SLGT1 inhibitor, phloridzin dihydrate (Millipore Sigma) was dissolved into 1M sucrose solution at a concentration of 0.4% w/v (8.47 mM) and was delivered like other stimulants for a final phloridzin concentration of ˜3 mM. Final stimulant concentrations were determined using the ratio of dilution and confirmed by osmolarity testing (Model 3320 Osmometer, Advanced Instruments Inc.). During 473 nm laser stimulation conditions, PBS perfusion was continuous, and the laser was pulsed for 1 minute at 40 Hz, 4 volts peak voltage, 20% duty cycle (473 nm, 80 mW laser, RGBlase).

Data Acquisition:

Extracellular voltage was recorded from a pair of platinum iridium wires as described above. A differential amplifier and bandpass filter (1000× gain, 300 Hz-5 kHz bandpass filter; A-M Systems LLC) was used and the signal was processed using a data acquisition board and software (20 kHz sampling rate; Signal Express, National Instruments Corp). The raw data was analyzed using a spike sorting algorithm (MATLAB by MathWorks). Spikes were detected using simple threshold detection based on RMS noise. The firing rate was calculated using a Gaussian kernel smoothing algorithm (200-ms time scale).

Statistical Methods:

Stimulation response was quantified as the maximum firing rate after stimulation (stimulant conditions) or during recording (baseline). Each trial serves as its own control by normalizing the maximum firing rate to the average baseline firing rate (defined as the first two minutes of recording, prior to stimulation onset). Mice were excluded from analysis if no sucrose response was present throughout recording. Maximum firing rate was analyzed across genotype, stimulation condition, and their interaction term by ANOVA, followed by HSD post hoc testing (iMP by SAS Institute).

Eating Behavior Assay.

Experimental (n=3), CckCRE_ChR2, or control (n=5) mice at 8-12 weeks old were used. An abdominal window was surgically implanted in order to gain access to the small intestine (distal duodenum/proximal jejunum). Mice were given 3 days to recover from the surgery and re-establish their normal eating behavior. Mice were then fasted overnight and the next day were anesthetized with 1-2% isoflurane and a laser was used to stimulate the small intestine through the window, either 437 nm for experimental, or 532 nm, or no laser, for the control (80 mW laser, RGBlase). The laser stimulation was pulsed at 40 Hz, 20% duty cycle for 30 minutes. Then mice were observed for 2 hours after recovery from anesthesia, while the number of food pellets eaten was recorded. Significance was determined using a two-tailed Student's t test.

Fast Blue Tracing.

Wild-type mice (6 weeks old) were given a 50 μl enema of Fast Blue (2 mg/ml). For vagotomy experiments, the mice were anesthetized and the right cervical vagus was cut. Mice were sacrificed 5 days post enema and tissue was collected and fixed for imaging.

Calcium Imaging.

For neurons, Phox2bCRE_GCaMP6s (n=3) nodose neurons were dissociated and plated. Nodose ganglia were dissected and digested in Liberase (Roche, 5401054001). Neurons were washed and filtered through a 70 μm cell strainer, then plated on 12 mm coverslips and placed in an incubator overnight. Cells were imaged 1-2 days later. Coverslips were placed in the recording chamber of a Zeiss Examiner Z1 and imaged with a Hamamatsu camera (Orca-flash4.0; C11440) using the Zeiss ZEN software package. For enteroendocrine cells, CckCRE_tdTomato organoids with transduced with ΔG-rabies-GCaMP6s. Organoids were imaged using multi photon microscopy 3-4 days after transduction. Imaging buffer (in mM: 140 NaCl, 5 KCl, 2 CaCl2, 2 MgCl2, 10 HEPES) was continuously perfused over the coverslips. Glucose (20 mM) was perfused for 30 seconds, then washed and KCl (40 mM) was applied for 30 seconds as an activity control. Images were analyzed with Fiji (it's just ImageJ), and cell traces were plotted with Matlab.

Single Cell Western Blot Analysis.

CckGFP cells were dissociated and cell sorted as outlined above. The Milo single-cell Western Blot (Protein Simple) was used according to the manufacturer's protocol and previously established technique (Hughes et al., Nature Methods 11:749 (2014)). Cells were loaded onto a micro-well gel by distributing the ˜40 k cell suspension onto the gel and through gravity cells settled into wells over 25 minutes. The chip was run through the Milo (10 sec lysis, 60 sec separation, followed by UV light for 3 min to stop the gel run). Gel was probed with two antibodies: 1-Rabbit anti-synapsin-1 (Cell Signaling #5297) at 1:10 dilution and Donkey anti-rabbit IgG Alexa 647 at 1:20 dilution; 2-Goat anti-GFP (Abcam # ab6673) at 100 μg/ml and Donkey anti-goat IgG Alexa 555 at 1:20 dilution. Chips were imaged with a microarray scanner and analyzed in Scout software (Protein Simple). Each lane was imaged in two channels and the area under the detected peak was used to quantify the target abundance in each cell.

Mass Spectrometry. Sample Preparation.

Equal amounts of Cck-GFP cells (˜100K cells/n) and non-GFP intestinal epithelial cells were sorted and collected in PBS. Nodose neurons, from both left and right ganglia, of the same mouse were also collected. Cells were spun down, PBS removed and snap frozen in liquid nitrogen. Frozen cells and tissue (n=3 per group) were thawed, and 50 μl of 0.25% ALS-1 in 50 mM ammonium bicarbonate, pH 8 was added to each sample followed by sonification. After centrifugation at 15,000×g for 10 min, the supernatant was transferred and protein recovery was quantified by Branford assay. After normalization of protein concentration with lysis buffer, samples were denatured and reduced by addition of 10 mM DTT and heated at 80° C. for 10 min, followed by alkylation with 20 mM iodoacetamide in the dark for 30 min. Sequencing grade modified trypsin (Promega; 1:50 w/w trypsin:protein) was added, and proteins were digested at 37° C. overnight. Samples were then acidified with 1% trifluoroacetic acid (TFA), 2% acetonitrile (CAN), followed by heating at 60° C. for 2 h, to inactivate trypsin and degrade ALS-1. Trypsinized yeast alcohol dehydrogenase 1 (MassPrep, Waters) was added at 50 fmol per μg as an internal standard to each sample. Following centrifugation, supernatants were transferred to Maximum Recovery LC vials (Waters). QC pools were prepared by mixing equal volumes of all samples.

Quantitative Mass Spectrometry.

Quantitative one-dimensional liquid chromatography, tandem mass spectrometry (1D-LC-MS/MS) was performed on 250 ng of the peptide digests per sample in singlicate, with additional QC and conditioning analyses. Samples were analyzed using a nanoAcquity UPLC system (Waters) coupled to a QExactive Plus high resolution accurate mass tandem mass spectrometer (Thermo) via a nanoelectrospray ionization source. Briefly the sample was first trapped on a Symmetry C18 300 mm×180 mm trapping column (5 μl/min at 99.9/0.1 v/v H2O/MeCN) followed by an analytical separation using a 1.7 μm Acuity HSS T3 C18 75 mm×250 mm column (Waters) with a 90-min. gradient of 5 to 40% CAN with 0.1% formic acid at a flow rate of 300 nl/min and column temperature of 55° C. Data collection on the QExactive Plus mass spectrometer was performed in data-dependent acquisition (DDA) mode of acquisition with an r=70,000 (@ m/z 200) at a target ΔGC value of 5e4 ions. A 20 s dynamic exclusion was employed. The total analysis cycle time for each sample injection was approximately 2 h.

Following 18 total analyses (including conditioning and QC injections), data was imported into Rosetta Elucidator v3.3 (Rosetta Biosoftware, Inc), and all LC-MS runs were aligned based on the accurate mass and retention time of detected ions (“features”) using Peak-Teller algorithm in Elucidator. Relative peptide abundance was calculated based on area-under-the-curve (AUC) of aligned features across all runs. The overall dataset had 221,597 quantified features, and 610,511 high collision energy (peptide fragment) spectra that were subjected to database searching. This MS/MS data was searched against a custom Swiss-prot database with Mus musculus taxonomy (downloaded on 01/27/15) with additional proteins, including yeast ADH and enhanced GFP, as well as an equal number of reversed-sequence “decoys” for false discovery rate determination (33,404 total entries). Mascot Distiller and Mascot Server (v2.5 Matrix Sciences) were utilized to produce fragment ion spectra and to perform the database searches. Included in the database searches were fixed modification on Cys (carbamidomethyl) and variable modifications on Met (oxidation) and Asn/Gln (deamidation). After individual peptide scoring using PeptideProphet algorithm in Elucidator, the data was annotated at a 0.8% peptide false discovery rate. For quantitative processing, the data was first curated to contain only high quality peptides with appropriate chromatographic peak shape and the dataset was intensity scaled to the robust mean across all samples analyzed.

iGluSnfr.

HEK 293/17 cells (ATCC CRL-11268) were maintained in DMEM (Gibco 11965) supplemented with 10% FBS (Corning 35-010-CV), Glutamax, and non-essential amino acids (both Gibco). Cells were transfected with 1 ug plasmid pCMV(MinDis) iGluSnFR (Addgene 41732) using 1 ul Lipofectamine 2000 in Optimem (Gibco) in 24-well plates.

iGLuSnFR Stable Cell Line.

The 5.2 Kb iGluSnFR (pCMV(MinDis).iGluSnFR was a gift from Loren Looger; Addgene plasmid #41732) was moved to the zeocin resistant backbone of Addgene #51694 (DRH296: FCK-Optopatch2 was a gift from Adam Cohen) as a NdeI+XhoI fragment. The resulting plasmid (pMEK8) was transfected into HEK 293/17 cells (ATCC CRL-11268) using 1 μl Lipofectamine 2000/ug DNA. Cells were maintained in DMEM (Gibco 11965) supplemented with 10% FBS (Corning 35-010-CV), Glutamax, non-essential amino acids (both Gibco) and 3 μl/ml zeocin (100 ng/ml, Invitrogen).

iGluSnFR-HEK Cell and Enteroendocrine Cell Co-Culture, and Imaging.

CckCRE_tdTomato enteroendocrine cells were isolated as described above. Isolated cells were mixed with iGluSnFR-HEK cells at a ratio of 10:1, then plated on 1% Matrigel coated coverslips. Control iGluSnFR-HEK cells were plated alone. Cells were incubated for 12-18 hours before imaging. Coverslips were imaged using a multiphoton microscopy system (Bruker Ultima IV with a Chameleon Vision II tunable laser). Imaging frames were captured at 1.876 fps, using 920 nm light. Imaging series were analyzed using Fiji (ImageJ), and cell traces were plotted with Excel.

Antagonist Studies.

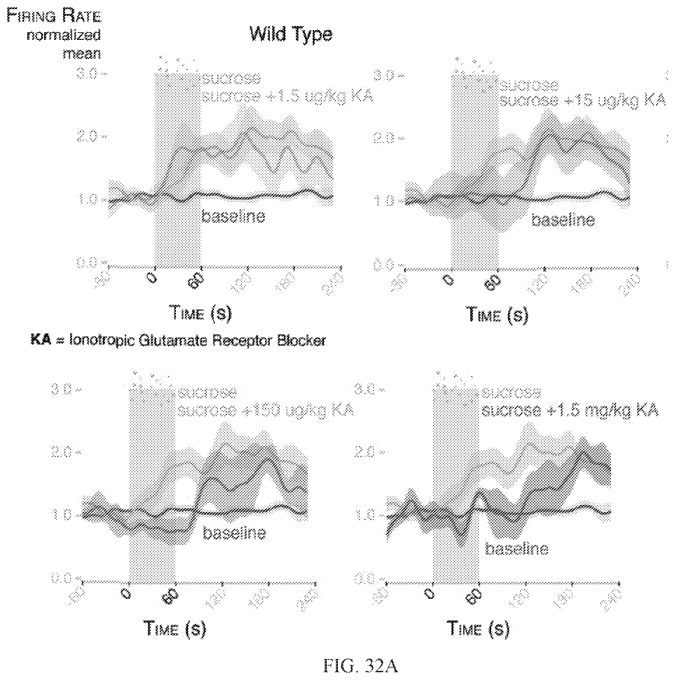

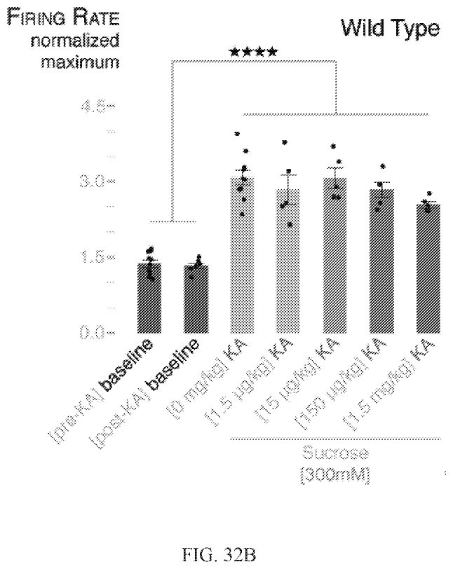

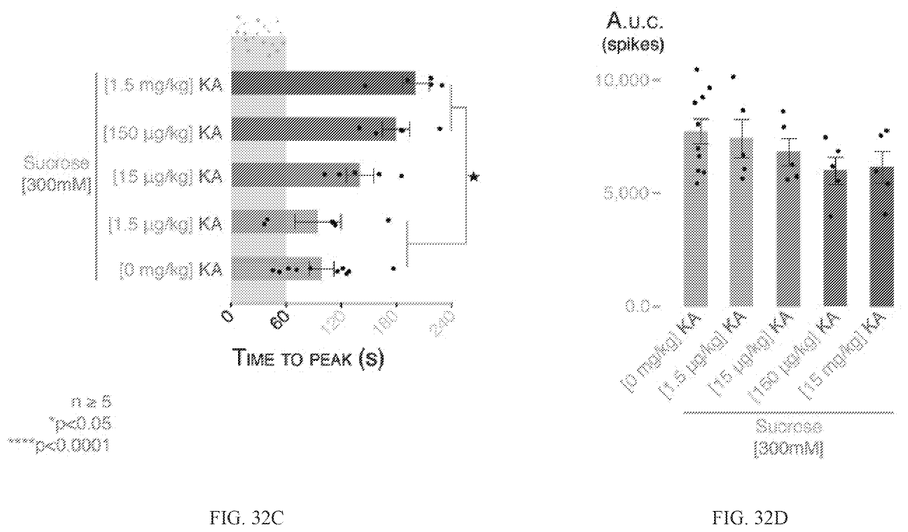

To observe the effects of phloridzin dehydrate (Sigma), devazepide (Sigma), kynurenic acid (Sigma), and DL-2-Amino-3-phosphonopropionic acid (AP-3, Sigma) on vagal nerve firing rate, response to sucrose, 473 nm laser, or CCK was recorded before and after antagonist delivery. All antagonists were delivered intraluminally over 1 minute in 200 μL solution via the gavage needle inserted into the duodenum, noting the duodenal perfusion exit incision released all inhibitors into the peritoneal cavity. For experiments using the SLGT1 inhibitor, phloridzin was dissolved into 1M sucrose solution at a concentration of 0.4% w/v (8.47 mM) and was delivered like other stimulants for a final phloridzin concentration of ˜3 mM. For experiments using devazepide the CCK-A receptor antagonist, devazepide was dissolved in DMSO and diluted in PBS for a final dose of 2 mg/kg in 200 μL solution. To validate the dose based on previously published reports, devazepide dose was titrated to fully attenuate vagal response to CCK (870 nm, 10 μg/kg); a dose of 1 mg/kg did not fully attenuate the response. Following a 5-minute incubation period, stimulant recordings continued for up to 1 hour with no return of CCK response. For experiments using the glutamate receptor antagonists, stock solutions of kynurenic acid and AP-3 were dissolved in 1M NaOH, and experimental concentrations were diluted in PBS, pH=7.4. Doses were determined based on previously published results: 1 mg/kg AP-3 and 150 μg/kg kynurenic acid. A dose response curve was acquired for kynurenic acid 1.5 μg/kg-1.5 mg/kg validating the chosen dose (FIGS. 32A-32D). For glutamate cocktail experiments, 200 μL of each inhibitor were delivered simultaneously for a total volume of 400 μL. Following dose, stimulant recording experiments were initiated immediately. Effect of inhibitor persisted 10-15 minutes. Therefore, each post-inhibitor data point for the glutamate inhibitors was from the recording directly following glutamate inhibitor application. Inhibitors were re-dosed as necessary throughout recording.

### Example 2

### Enteroendocrine Cells Contact Sensory Nerves

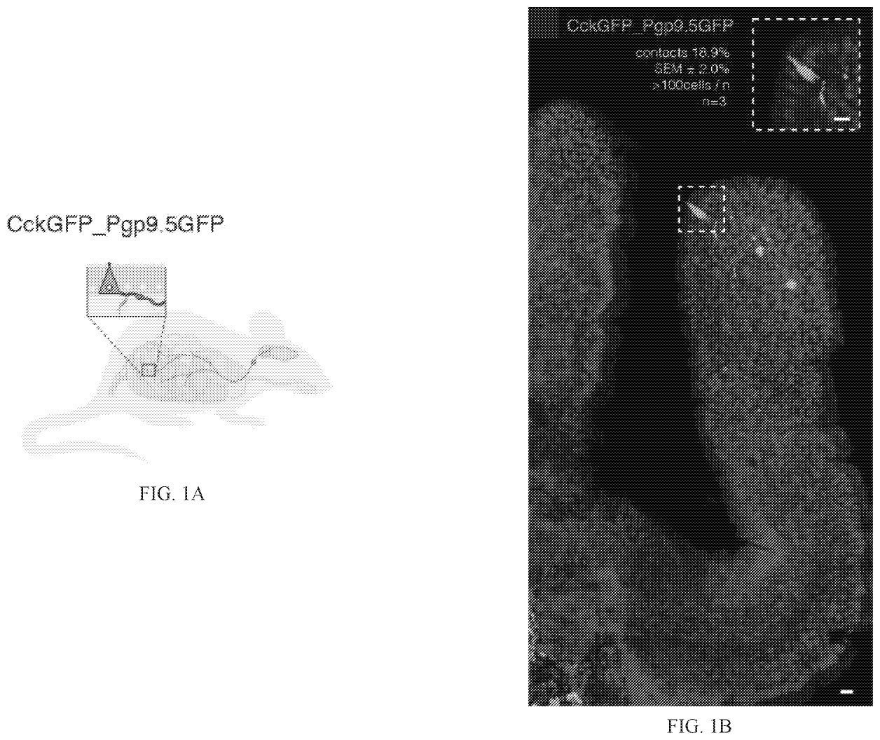

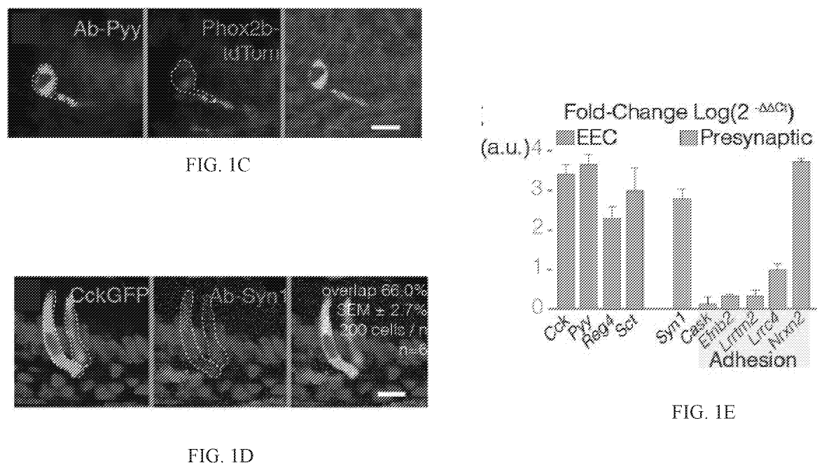

In the mouse small intestine, enteroendocrine cells were seen contacting sensory nerve fibers (18.9%±2.0% S.E.M.; >100 cells per n; n=3) (FIGS. 1A-1C). Specifically, PGP9.5-sensory nerve fibers are seen contacting CCK-enteroendocrine cells. Cholecystokinin (CCK) and Peptide YY (PYY) were used in the following studies as markers of enteroendocrine cells. Recent reports showed that a single enteroendocrine cell expresses multiple neuropeptides, including both CCK and PYY, which was confirmed using mass spectroscopy analysis (Table 3). Table 3 shows gut neuropeptides with high expression in intestinal Cck-GFP sorted cells compared to non-GFP intestinal epithelial cells (n=3 mice). Using quantitative mass spectrometry, purified CCK cells were found to contain significant amounts of at least 10 other neuropeptides including peptide YY protachykinin-1, and tryptophan hydroxylase 1 (Cck-GFP>10-fold compared to non-GFP epithelial cells; p<0.05; n-3). The last two proteins are often used as markers of the enterochromaffin cell subtype. Therefore, the same enteroendocrine cell can secrete multiple neuropeptides, including both CCk and PYY, which were used as markers.

TABLE 3
Proteins with high expression in CCK-positive sorted cells compared to negative epithelial cells.

| | | Number of | | | |
|---|---|---|---|---|---|
| Primary protein | | quantified | % CV QC | Fold- | p-value |
| name | Protein Description | peptides | pools | Change | t-test |
| GLUC_MOUSE | Glucagon GN = Gcg PE = 1 SV = 1 | 13 | 6.81 | 207.95 | 2.09E−06 |
| GIP_MOUSE | Gastric inhibitory polypeptide | 20 | 2.29 | 194.03 | 4.20E−04 |
| | GN = Gip PE = 2 SV = 2 | | | | |
| GHRL_MOUSE | Appetite-regulating hormone | 6 | 0.53 | 163.38 | 8.42E−04 |
| | GN = Ghrl PE = 1 SV = 1 | | | | |
| CCKN_MOUSE | Cholecystokinin GN = Cck PE = 1 | 6 | 3.05 | 145.41 | 3.00E−03 |
| | SV = 3 | | | | |
| TKN1_MOUSE | Protachykinin GN = Tac1 PE = 2 SV = 1 | 2 | 13.13 | 142.26 | 1.64E−05 |
| SECR_MOUSE | Secretin GN = Sct PE = 2 SV = 1 | 8 | 1.71 | 71.19 | 7.50E−05 |
| SMS_MOUSE | Somatostatin GN = Sst PE = 2 SV = 1 | 3 | 5.85 | 51.53 | 4.37E−04 |
| NEUT_MOUSE | Neurotensin/neuromcdin GN = Nts | 8 | 9.75 | 30.82 | 1.06E−04 |
| | PE = 2 SV = 1 | | | | |
| CART_MOUSE | Cocaine- and amphetamine- | 2 | 7.10 | 20.67 | 2.00E−03 |
| | regulated transcript protein | | | | |
| | GN = Cartpt PE = 2 SV = 2 | | | | |
| PYY_MOUSE | Peptide YY GN = Pyy PE = 2 SV = 3 | 4 | 2.36 | 17.96 | 3.14E−05 |
| TPH1_MOUSE | Tryptophan 5-hydroxylase 1 | 1 | 20.66 | 14.48 | 3.6E−02 |
| | GN = Tph1 PE = 2 SV = 1 | | | | |

N = 3 mice

These connections with nerves had synaptic features as they immunoreacted with an antibody for the pre-synaptic protein Synapsin-1 (FIG. 1D). Using single-cell western blot, 83% of enteroendocrine cells were found to contain synapsin-1 (164 of 198 Cck-GFP cells analyzed) (FIG. 5). Moreover, compared to other intestinal epithelial cells, purified CCK enteroendocrine cells expressed Efnb2, Lrrtm2, Lrrc4, and Nrxn2—all synaptic adhesion genes (FIG. 1E). Thus, the enteroendocrine cells had the necessary synaptic machinery to engage in neurotransmission with sensory nerves.

### Example 3

### A Gut-Brain Monosynaptic Neural Circuit

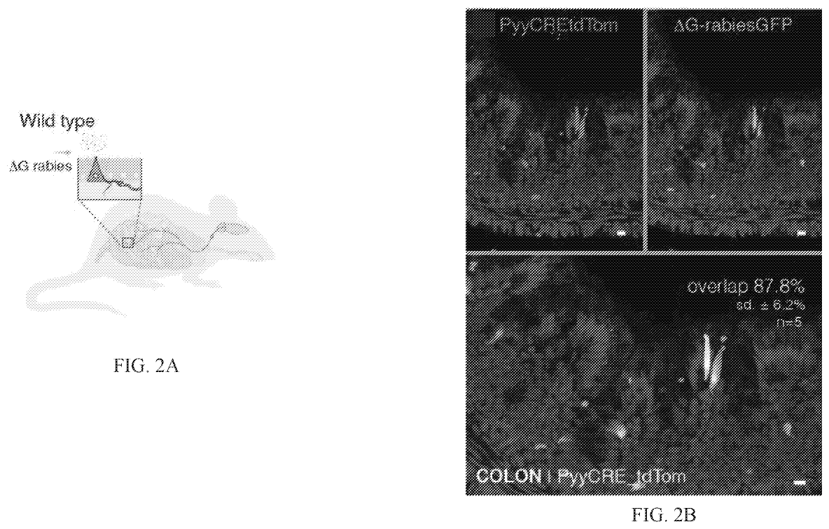

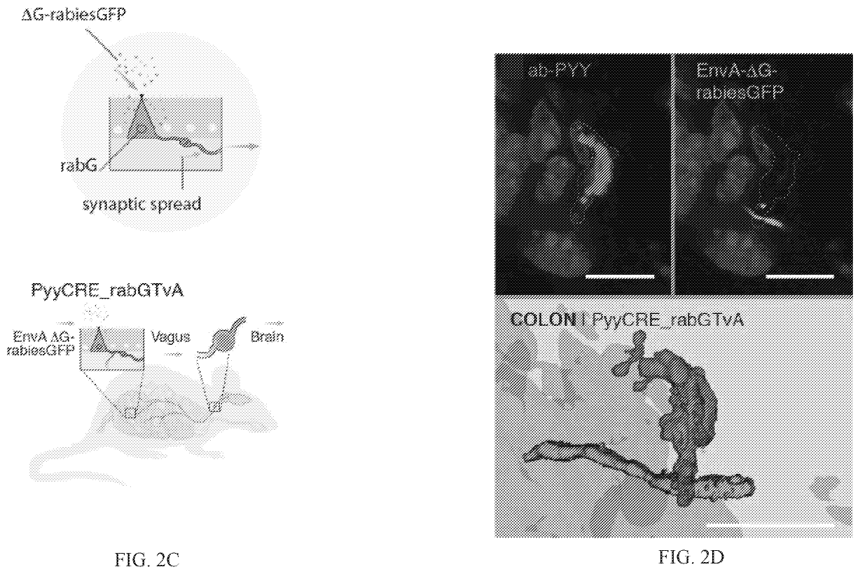

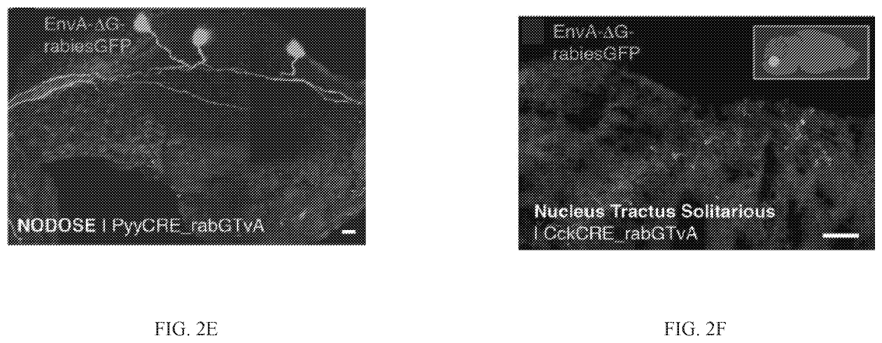

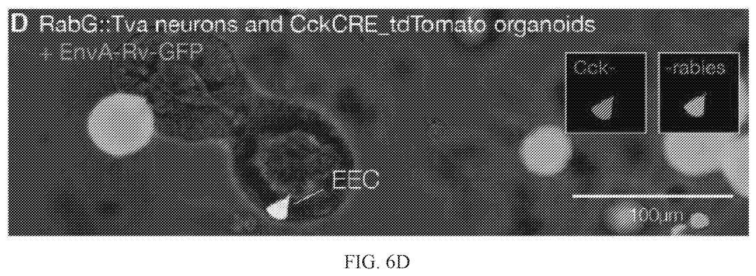

A synapse connecting the gut lumen to the brain stem was discovered as follows. To determine the source of neurons synapsing with enteroendocrine cells, a monosynaptic rabies virus (ΔG-rabies-GFP) was used. This rabies infected neurons, but lacked its glycoprotein G necessary for trans-synaptic spread (FIG. 2A). Compared to other intestinal epithelial cells, this rabies infected enteroendocrine cells almost exclusively as observed in vitro in intestinal organoids (FIG. 6A). Moreover, if introduced into the lumen of the mouse colon by enema, this rabies virus infected almost 9 in 10 enteroendocrine cells (87.8%±SEM 6.2, n=5) (FIG. 2B). The conditions of the experiment, including mouse age as well as volume and concentration of viral load were previously optimized (Bohorquez et al. J. Clin. Invest 125: 782-786 (2015)). In the absence of its G glycoprotein the rabies virus did not spread beyond infected enteroendocrine cells as shown by the lack of fluorescence in the underlying mucosa.

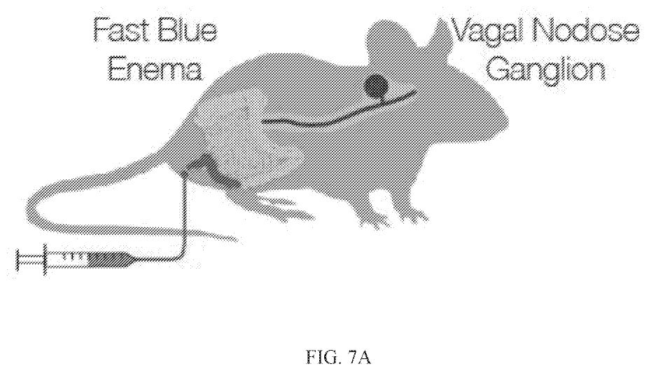

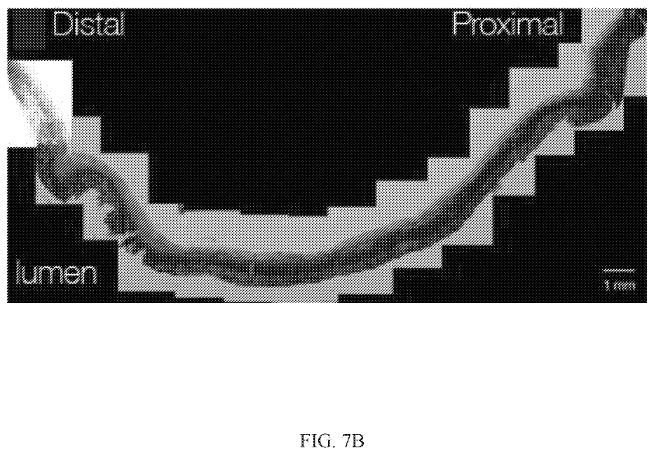

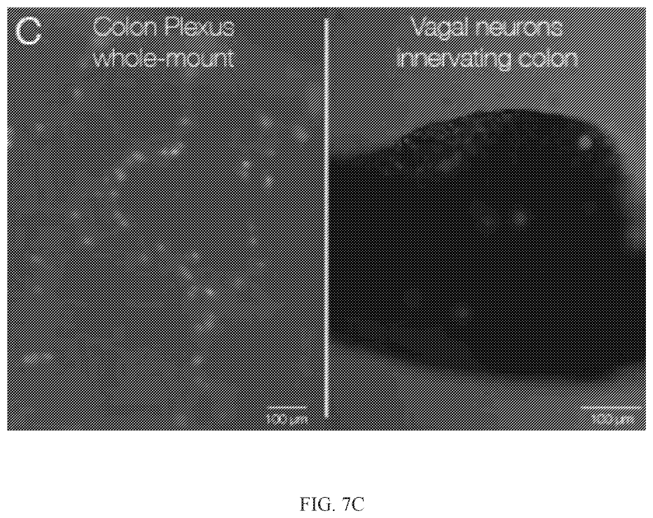

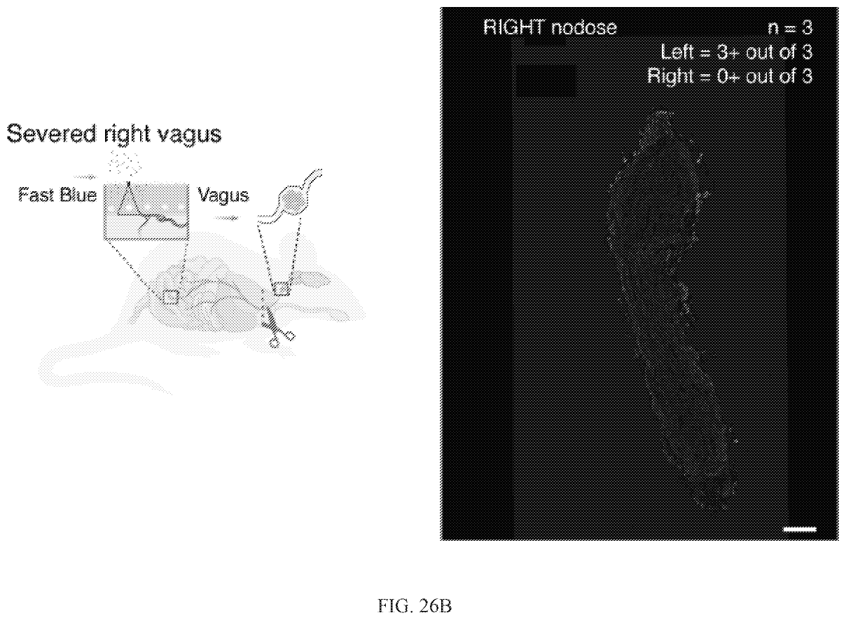

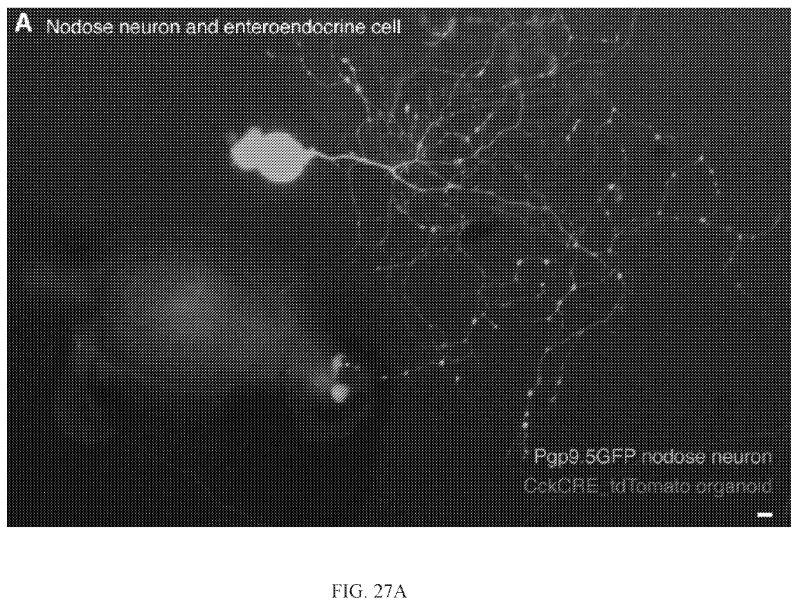

Next, to trace the neural circuit, a mouse (PyyCRE_rabG-TvA) was bred in which enteroendocrine cells express the glycoprotein G (FIG. 2C). In these mice, rabies delivered by enema infected enteroendocrine cells and spreads trans-synaptically to nerves. Some of the nerve fibers could be traced up to neurons of the vagal nodose ganglia (control: n=0+ out of 3 PyyCRE_tdTomato; experimental: n=4+ out of 5 PyyCRE_rabG-TvA mice). An enema of the chemical tracer fast blue dye confirmed that vagal nodose neurons indeed innervated the distal colon (FIG. 7). In control experiments, the right cervical vagus was severed (FIG. 26B). A Fast-Blue enema failed to label neurons in the right nodose (vagotomized), but not the left nodose (FIG. 26A and FIG. 26B). It was possible that ΔG-rabies-GFP could infect any neuronal cell that it contacts. Therefore, its entrance to enteroendocrine cells only was further restricted by using an EnvA coated rabies (FIG. 2C).

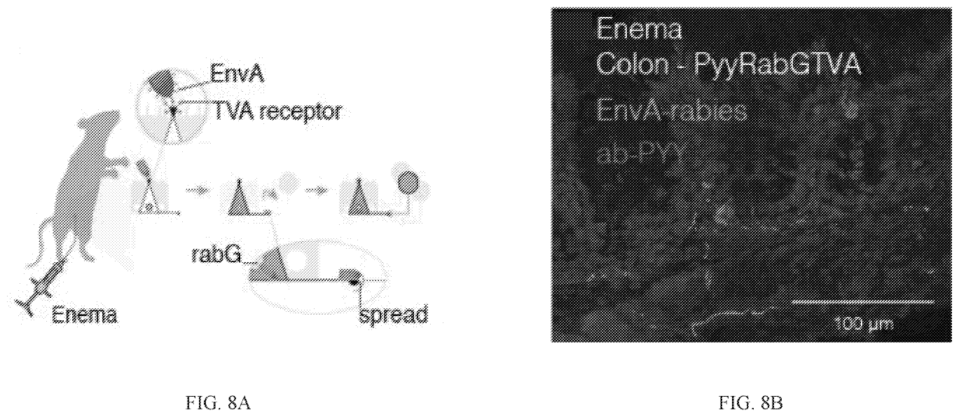

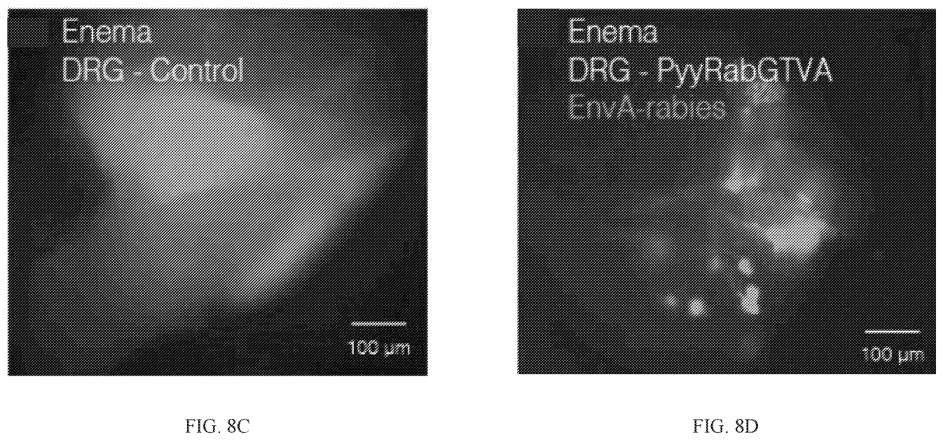

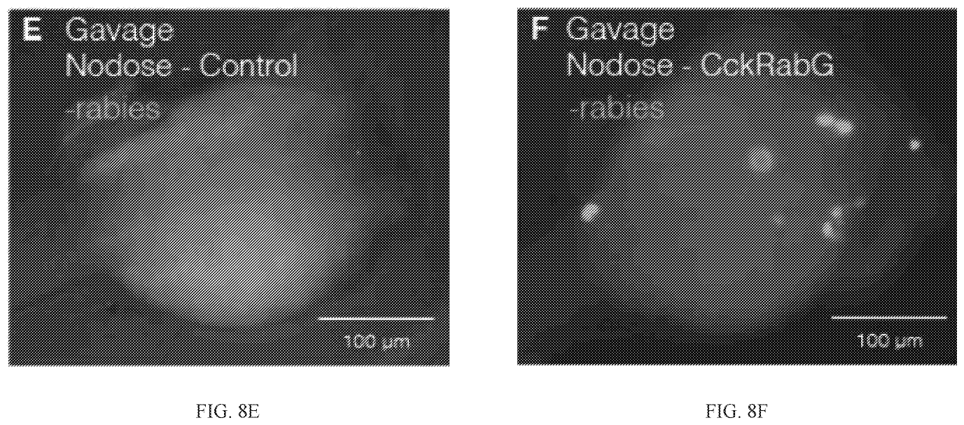

EnvA is an envelope glycoprotein of the avian sarcoma leukosis virus that binds to the avian TvA receptor. By coating the ΔG-rabies-GFP with EnvA, rabies infection was restricted to cells that expressed a TvA receptor. In the PyyCRE_rabG-TvA mouse, enteroendocrine cells expressed the TvA receptor, and an enema of the EnvA rabies infected enteroendocrine cells exclusively, then spread to nerves. Of a total 9 mice, 5 had visible labeling of nerve fibers in the colon (FIG. 2D) and 2 in nodose ganglia neurons (n=2+ out of 5 with colon transduction) (FIG. 2E; confirmed in vitro FIGS. 6B-6D). Neurons were observed labeled in the dorsal root ganglia of 4 out of 5 transduced mice (FIG. 8). No infection in nerves was observed in littermate controls rabG-TvA mice that lack CRE recombinase (n=5).

Similar results were confirmed when the virus was delivered by oral gavage into CckCRE_rabGTvA mice (FIG. 8). In these mice, labeling of vagal neuron projections upstream of the nodose ended in the nucleus solitarius tract of the brainstem, determined by coordinates from the Allen Brain Atlas (FIG. 2F). EnvA rabies tracing showed a monosynaptic neural circuit linking the small intestine or colon lumen to the brainstem. Because rabies was a neurotrophic virus, these results also uncovered a potential path for pathogens in the gut lumen to gain access to the central nervous system.

This Gut-Brain Neural Circuit was Recapitulated In Vitro.

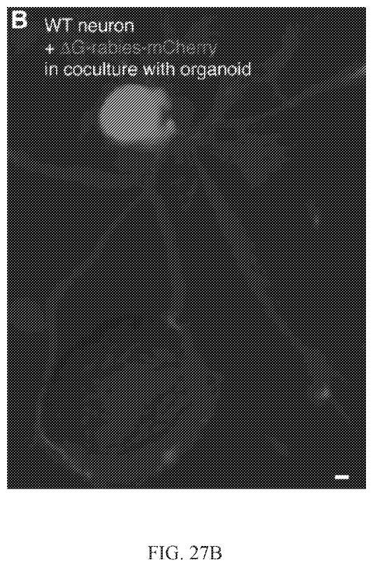

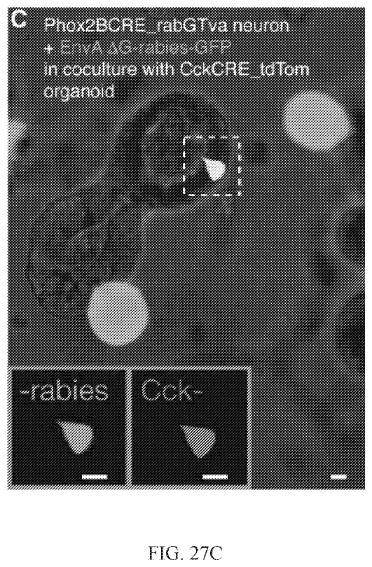

In co-culture, vagal nodose neurons clearly extended axons to enteroendocrine cells in intestinal organoids (FIG. 6B). Vagal nodose neuron axon extension to enteroendocrine cells in intestinal organoids was also visualized in a wide field view (FIG. 27A). To confirm that synapses were formed, the path in vitro was traced from vagal nodose neurons to enteroendocrine cells using EnvA rabies. To ensure that only transduced neurons could spread the EnvA rabies virus, nodose neurons were incubated with EnvA rabies virus prior to coculture with organoids. In control experiments, EnvA rabies did not infect wild-type nodose neurons (FIG. 27B). However, 48 to 72 hours after exposure, EnvA rabies infected TvA expressing vagal nodose neurons (Phox2BCRE_rabG-Tva) and spread onto enteroendocrine cells in intestinal organoids (FIGS. 6C-6D). Rabies infection of vagal nodose neurons and spread to enteroendocrine cells was also visualized in a wide field view (FIG. 27C). EnvA rabies neither infected control_rabG-Tva nodose neurons nor other cells in intestinal organoids (FIG. 6C). These data show that vagal nodose and enteroendocrine cells synapsed in vitro as shown by the trans-synaptic spread of EnvA rabies.

### Example 4

### Gut-Brain Sensory Transduction

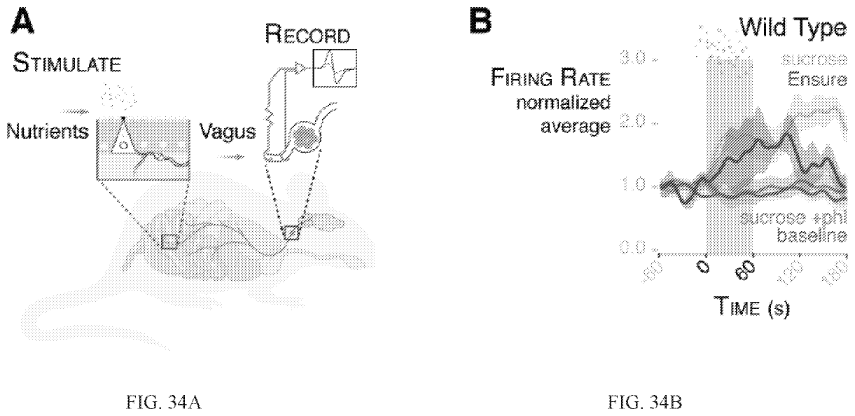

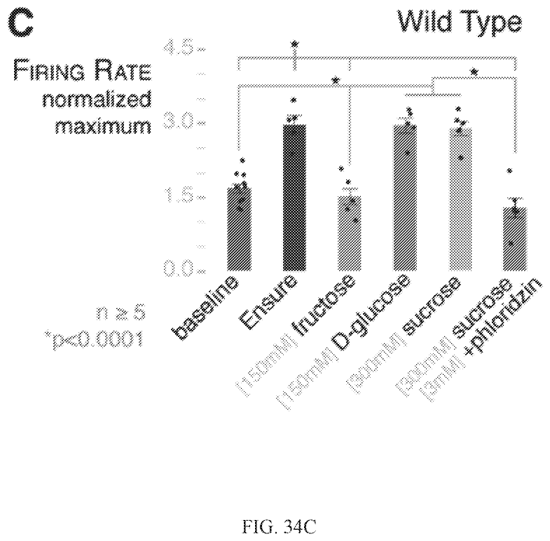

To test the function of this neuroepithelial circuit, a whole nutrient, ENSURE® was used. Whole nerve electrophysiology established that luminal ENSURE® stimulates vagal firing (FIGS. 34A-34C).

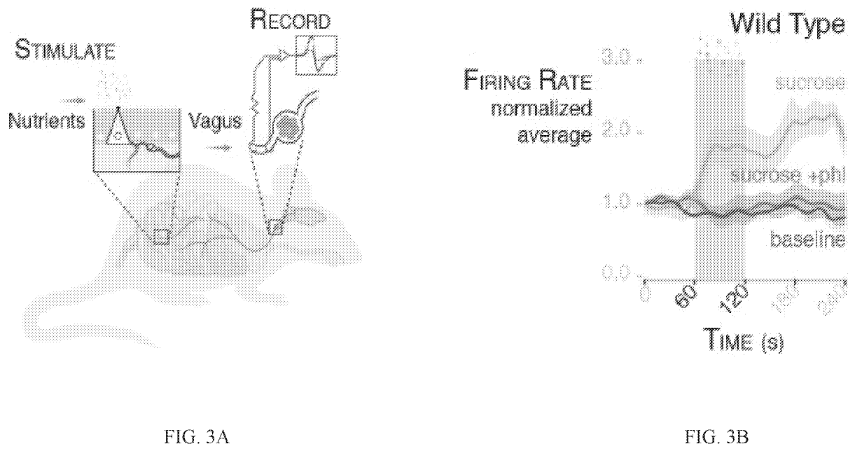

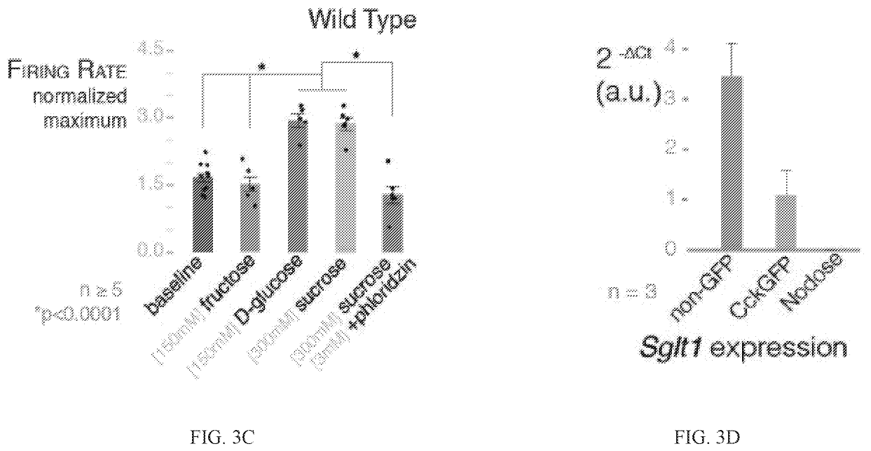

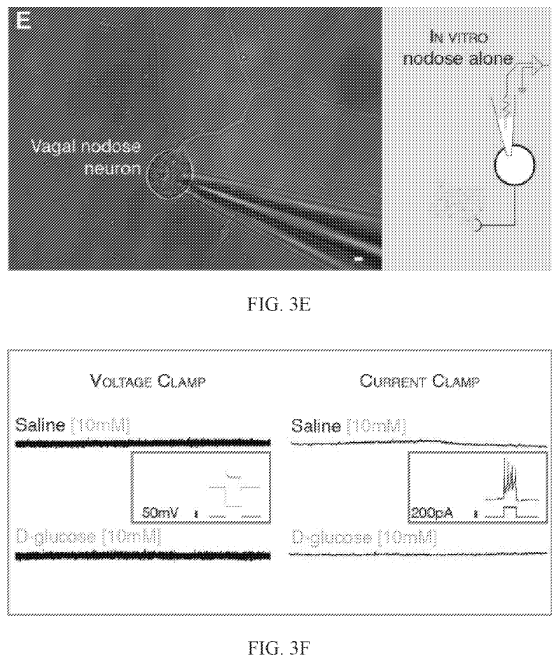

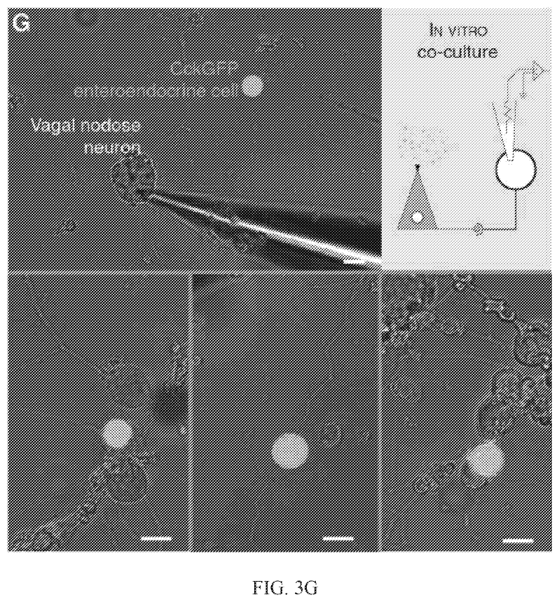

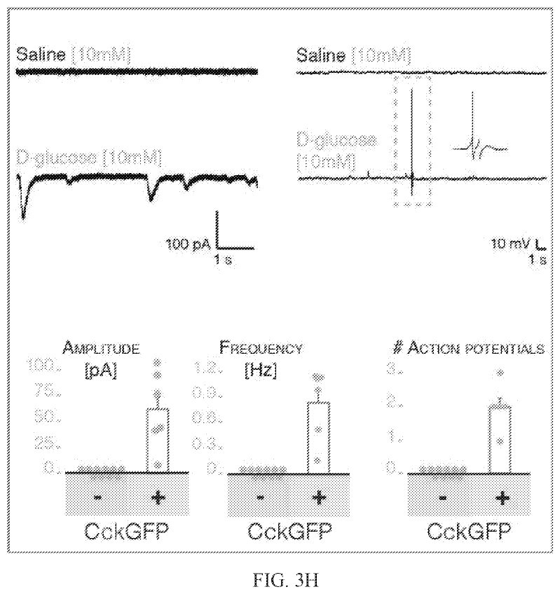

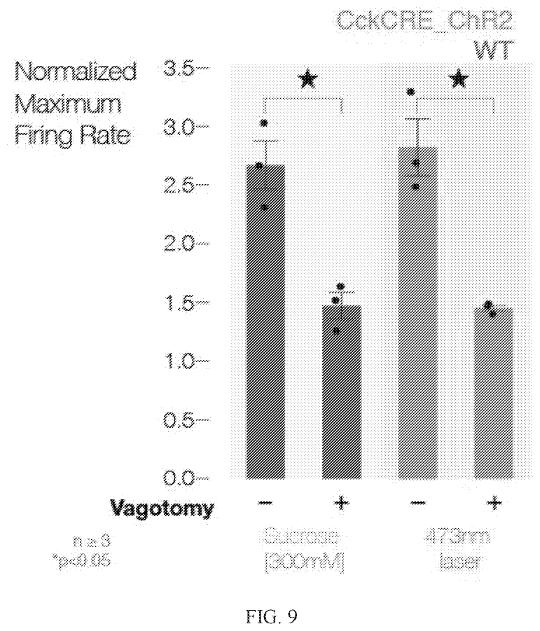

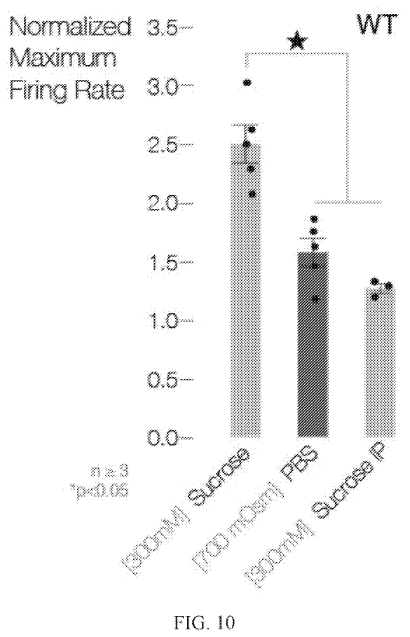

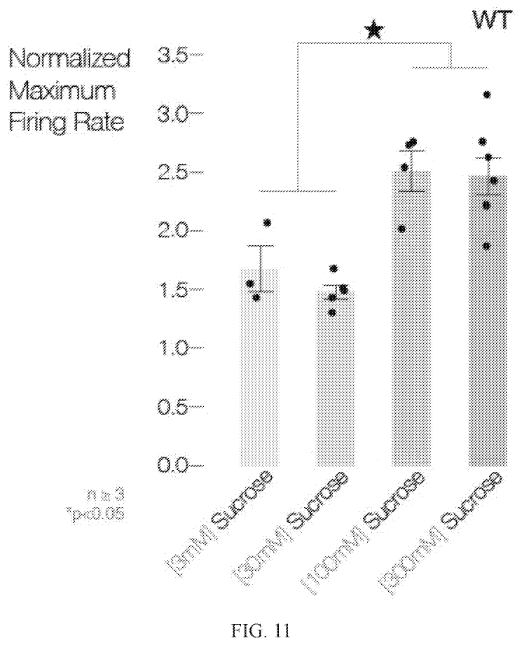

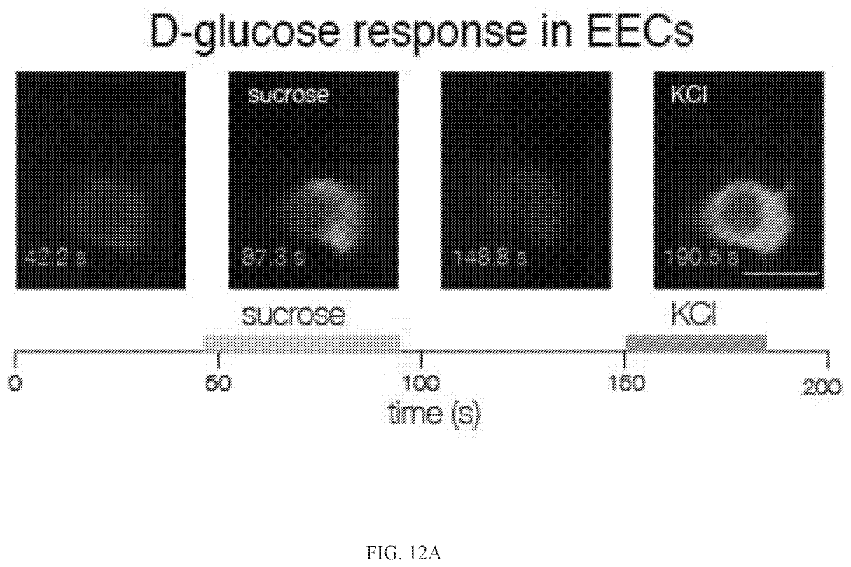

To further test the function of this neuroepithelial circuit, a distinctive nutrient, glucose, was used. Ingested glucose is known to be sensed in the duodenum, but it was unclear if enteroendocrine cells transduce the stimulus or the vagus senses it directly. Using whole nerve electrophysiology, luminal glucose stimulated vagal firing rate (FIGS. 3A-3C). In wild type mice, perfusing sucrose (100 mM-300 mM), a disaccharide formed by D-glucose and fructose, significantly increased vagal firing rate over baseline (FIG. 3B-C; FIG. 11). D-glucose (150 mM), but not fructose (150 mM), had the same effect. The effect was not observed when the vagus was severed (FIG. 9), and when hyperosmolar PBS (700 mOsm) or intraperitoneal sucrose (300 mM) was perfused (FIG. 10). The stimulus was abolished when sucrose was perfused along with phloridzin—a blocker of the electrogenic glucose transporter SGLT1 (FIGS. 3B-3C). A transcription profile showed that CCK enteroendocrine cells, but not vagal nodose neurons, express Sglt1 (FIG. 3D).

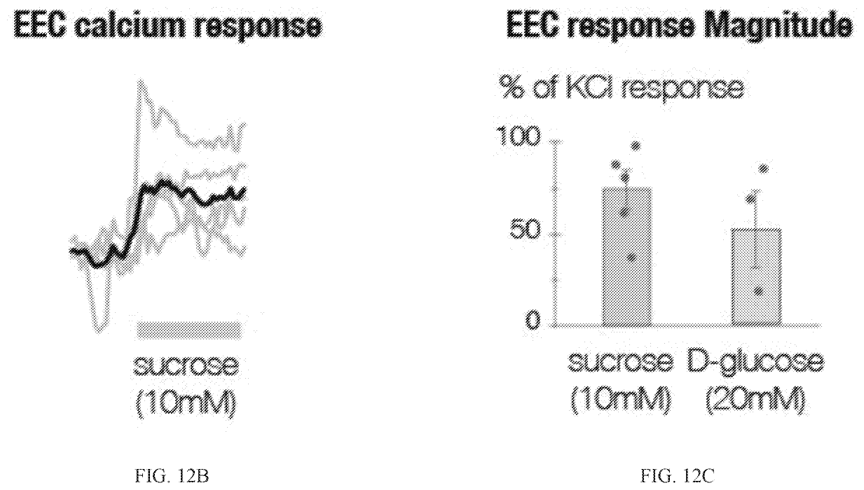

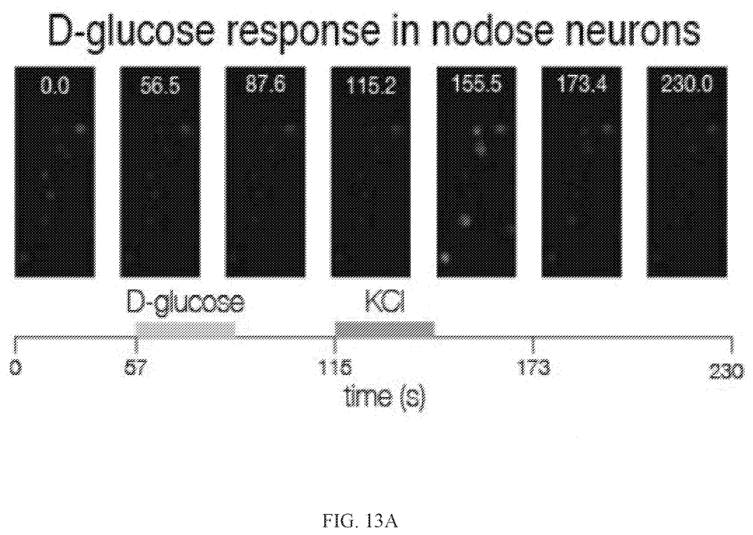

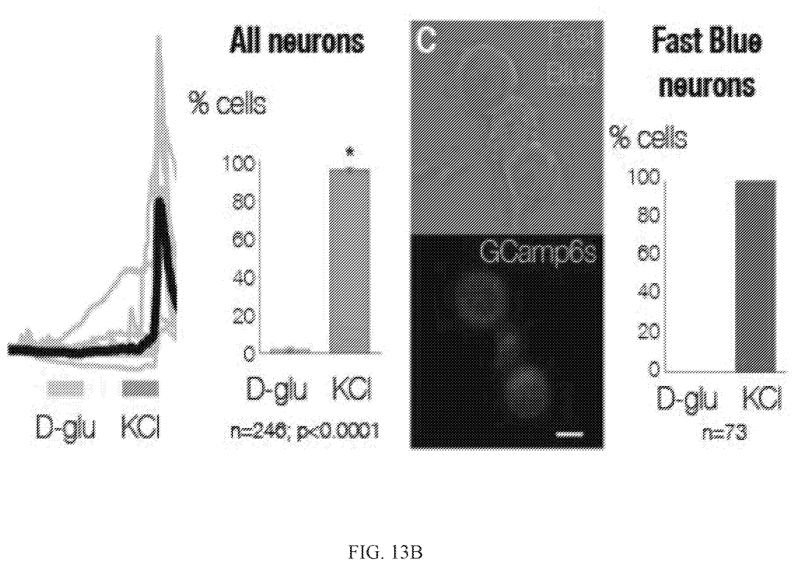

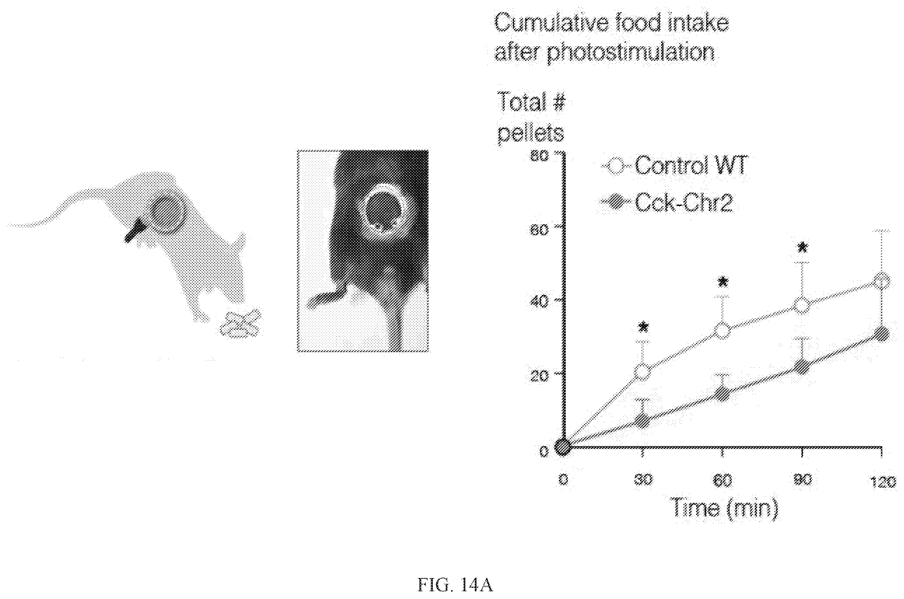

Evidence gathered on dissociated colonic enteroendocrine cells, and the enteroendocrine-like cell line STC1, showed that enteroendocrine cells sensed glucose. To confirm this finding, a rabies vector was developed to express the calcium reporter GCaMP6s in enteroendocrine cells from intestinal organoids. Indeed, when presented with D-glucose (10 mM), calcium transients were elicited in intestinal CCK enteroendocrine cells (56.0%±20.0% of the KCl control response; n=3) (FIG. 12). However, D-glucose (10 mM) did not elicit calcium transients in dissociated nodose ganglia neurons (FIG. 13) (n=246 cells pooled from 3 mice). This result contradicted one previous report in which rat nodose neurons appeared to respond to glucose. Unlike enteroendocrine cells, vagal neurons were unlikely to face steep glucose concentrations because they do not contact the intestinal lumen.

To discard the possibility that only nodose neurons innervating the intestine may sense glucose, they were retro-traced by injecting Fast Blue dye into the duodenum (FIG. 13C). In Fast Blue labeled neurons, no calcium response was observed in the presence of D-glucose (20 mM) (FIG. 13). Furthermore, neither excitatory currents nor action potentials were observed in the presence of a 10-20 mM D-glucose using patch clamp electrophysiology (FIGS. 3E and 3F). Current injection demonstrated that these cultured nodose neurons were functionally viable (FIG. 3F inset). Therefore, under the present conditions, D-glucose does not stimulate vagal nodose neurons directly.

Intestinal enteroendocrine cells were then co-cultured with vagal nodose neurons to test the transduction of glucose using a preparation previously described to show a single CCK enteroendocrine cell connecting to a single sensory neuron in vitro (Bohorquez et al. J. Clin. Invest 125: 782-786 (2015)). After co-culturing for 48 to 72 hours, there were visible connections between enteroendocrine cells and vagal nodose neurons (FIG. 3G). Using patch-clamp electrophysiology, D-glucose (10 mM) was found to evoke excitatory postsynaptic potentials and action potentials in vagal nodose neurons connected to enteroendocrine cells (FIG. 3H). In voltage-clamp, the average current of the excitatory post-synaptic potentials (EPSPs) was 61.65±15.21 pA with an average frequency of 0.86±0.17 Hz (n=6). In current-clamp, this in vitro connection was also sufficient to elicit action potentials in the connected neurons (average of 2±0.32, n=5 pairs). There was no difference in resting membrane potential, or current/spike threshold between neurons cultured alone, or with enteroendocrine cells. These results show that enteroendocrine cells synapse with vagal nodose to transduce glucose stimuli in vitro.

### Example 5

### Speed and Specificity of Transduction

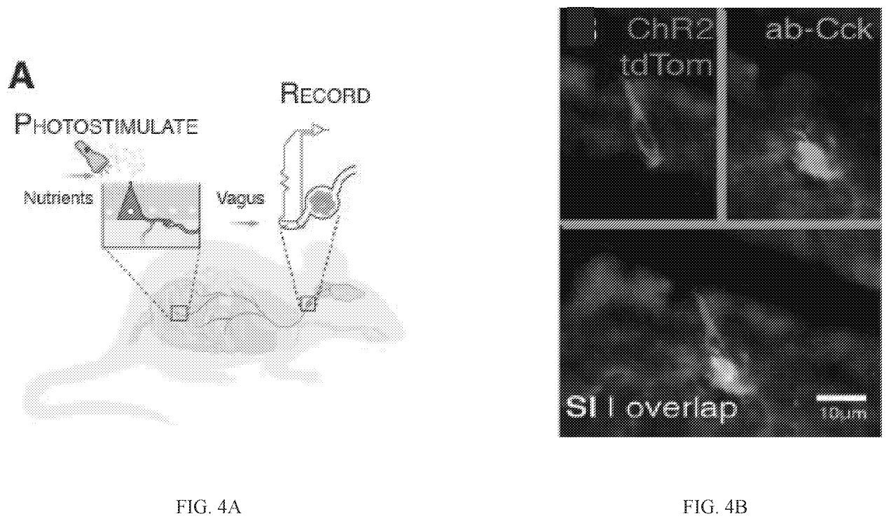

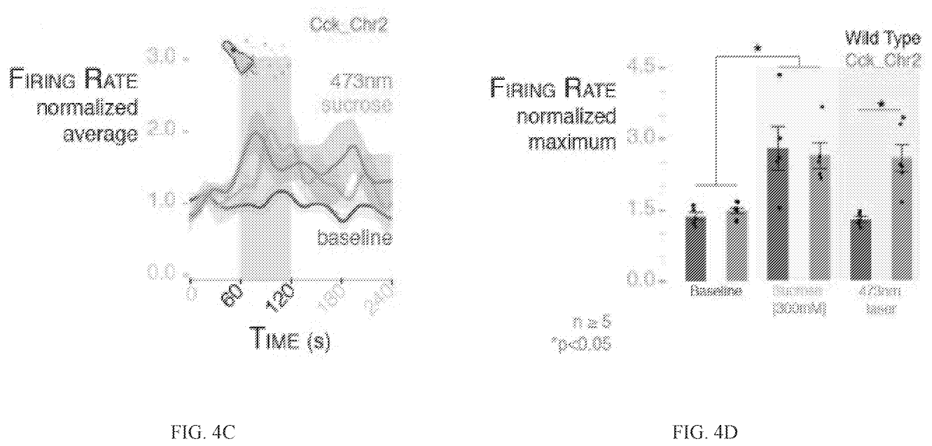

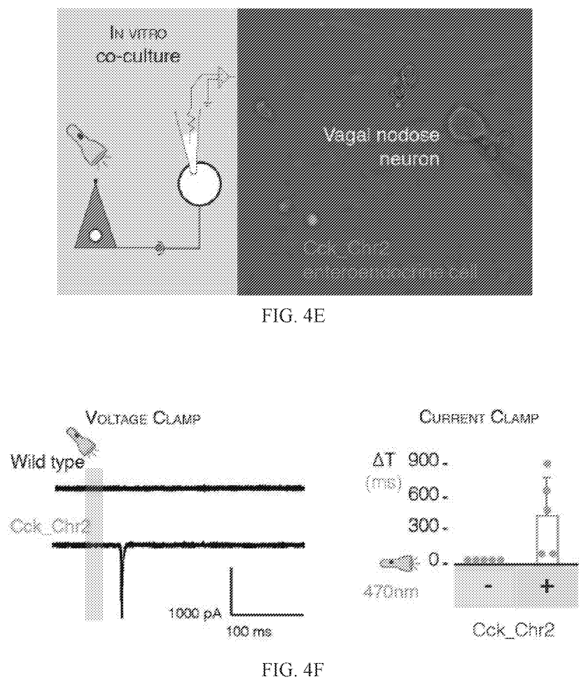

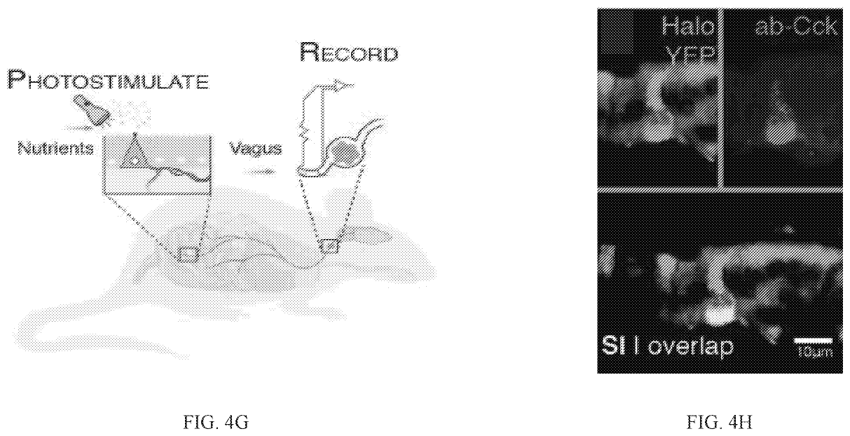

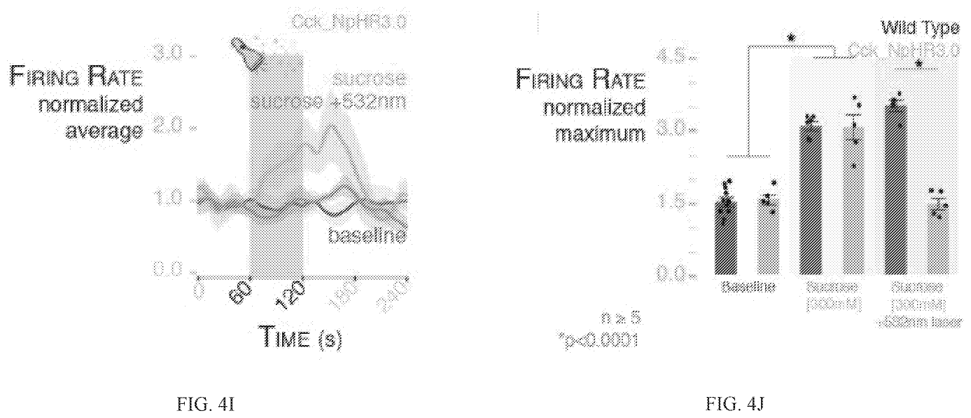

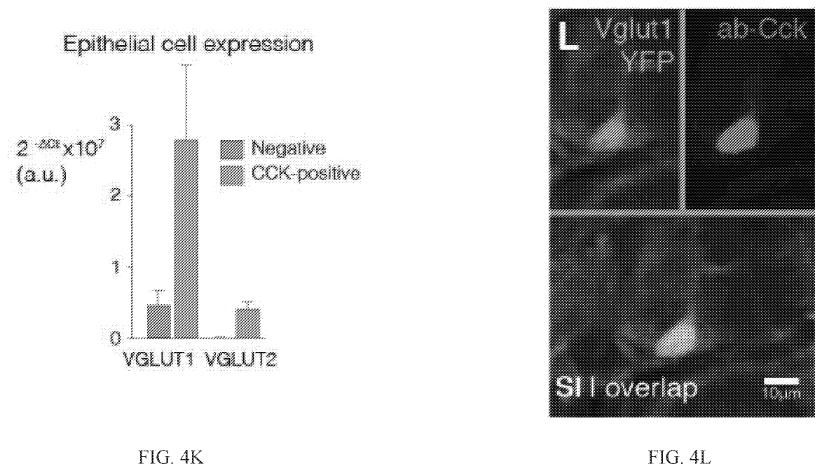

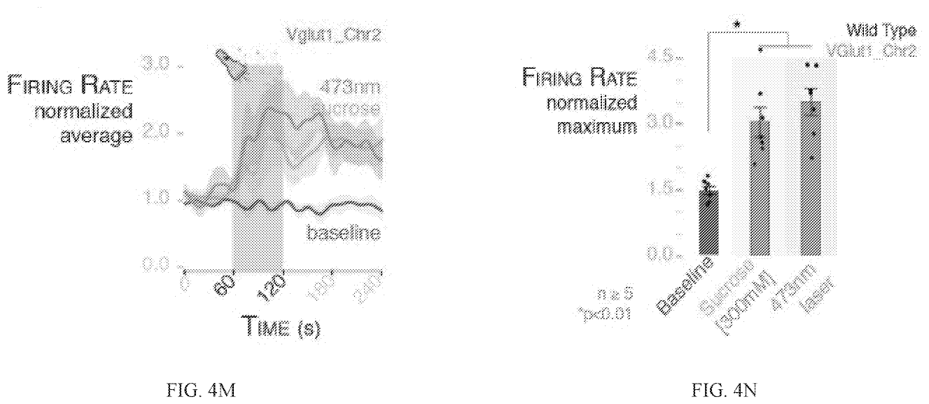

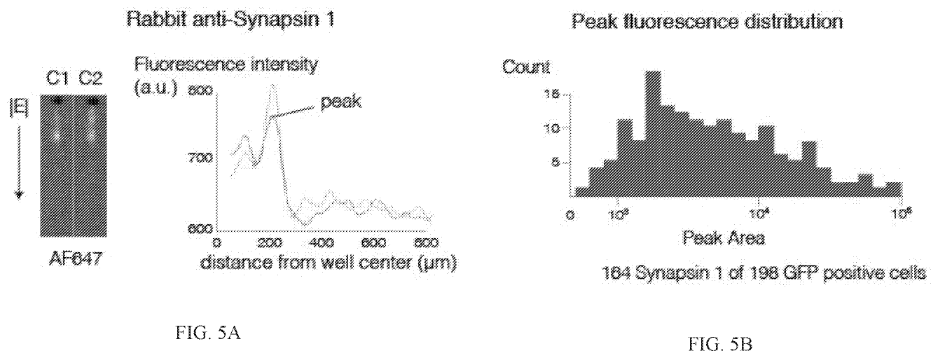

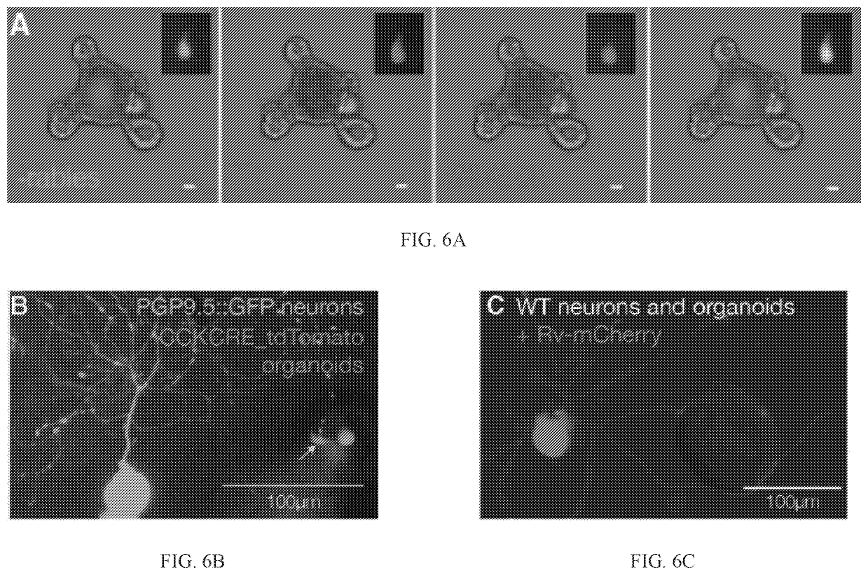

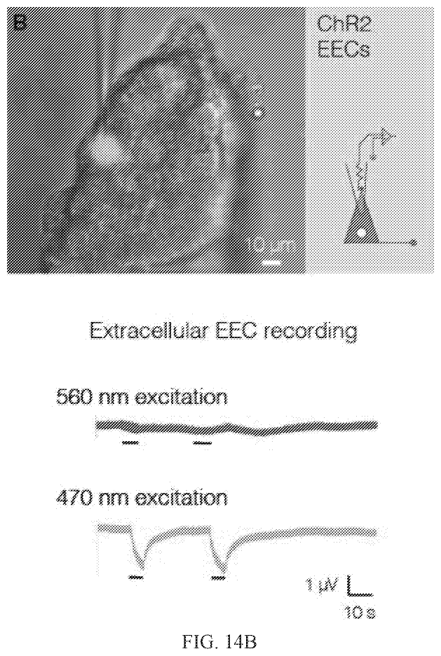

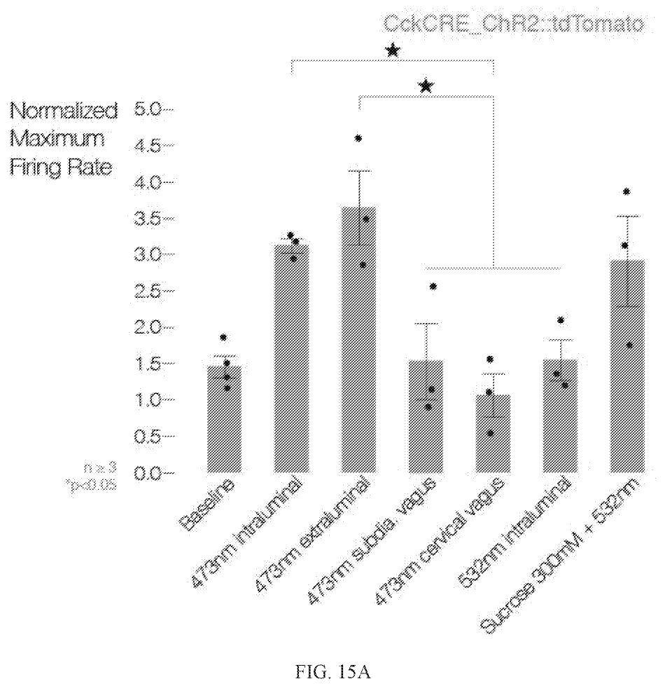

The development of optogenetics has made it possible to dissect in real time the contribution of an electrically excitable cell in a neural circuit. A mouse was bred (CckCRE_Chr2-tdTomato) in which enteroendocrine cells express Channelrhodopsin 2 (Chr2)—an excitatory light-gated ion channel activated by 473 nm light (FIGS. 4A-4B). In these mice, a 40 Hz stimulus of 473 nm laser light prior to the presentation of food significantly reduced food intake over a 2-hour period (P<0.05; n=3) (FIG. 14A); and, enteroendocrine cells from these mice showed excitatory currents in the presence of 470 nm light (FIG. 14B), indicating that Chr2 was functionally expressed in enteroendocrine cells. Further details of the experiment, including controls, is described in Example 1.

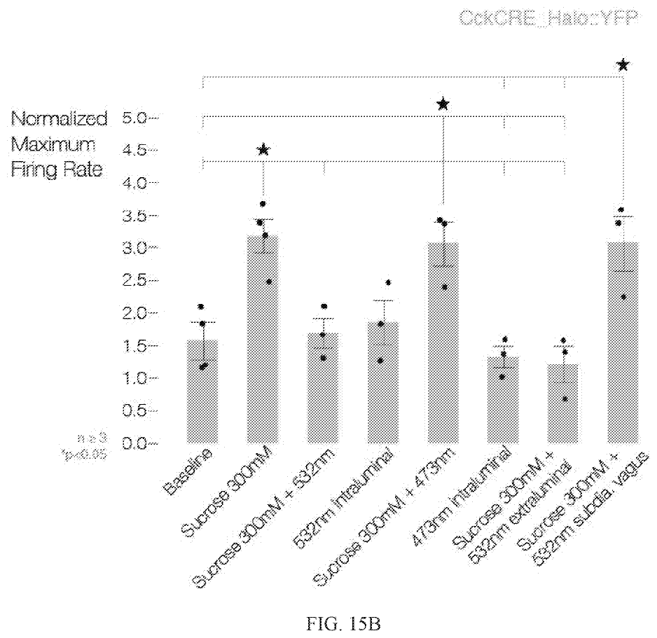

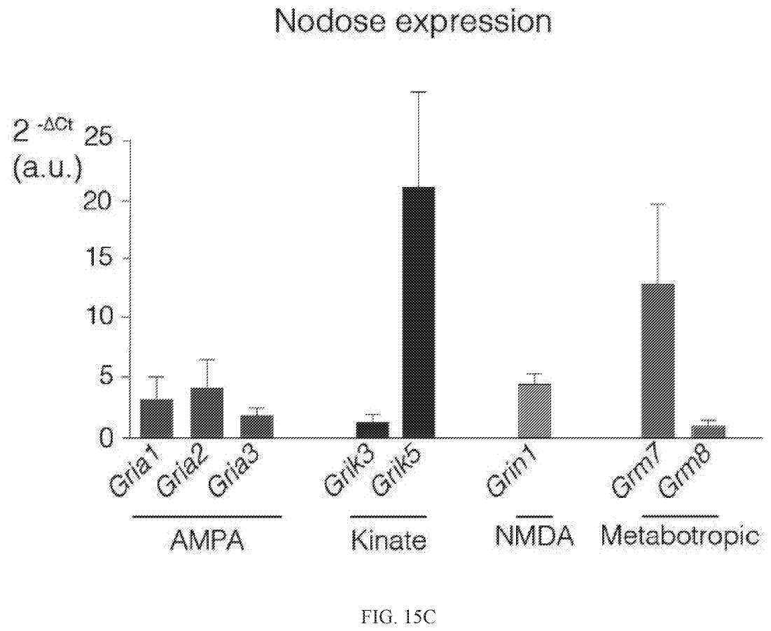

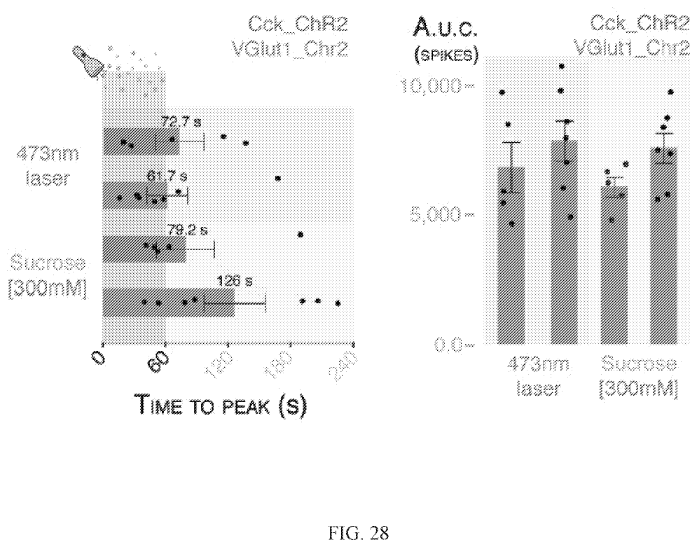

Moreover, a laser stimulus of 473 nm in the duodenal lumen of CckCRE_Chr2 mice, but not wild-type controls, significantly increased vagal firing rate (FIGS. 4C-4D; laser activation controls FIG. 15A). Vagal firing rate increased rapidly following laser stimulation, reaching peak firing rate in an average of 61.7 seconds (FIG. 28). To determine the transduction speed between enteroendocrine cell and neuron, Chr2 enteroendocrine cells were co-cultured with vagal nodose neurons (FIG. 4E). In connected pairs, a 470 nm light pulse elicited excitatory postsynaptic currents in vagal nodose neurons with a time delay ranging from 60-800 milliseconds (n=5 pairs) (FIG. 4F). Photo-stimulating the neurons cultured alone did not elicit any activity. These results show, both in vivo and in vitro, that enteroendocrine cell activation was sufficient to induce an excitatory response of vagal neurons.

To test the specificity, a mouse (CckCRE_Halo-YFP) was bred in which intestinal enteroendocrine cells expressed the light inhibitory channel Halorhodopsin (eNpHR3.0) (FIGS. 4G-4H). Halorhodopsin is a chloride ion channel that hyperpolarizes cells in the presence of 532 nm light. In these mice, luminal sucrose (300 mM) elicited a vagal response, but when sucrose was presented along with a 532 nm laser stimulus, vagal activity was abolished (FIGS. 4I-4J; laser activation controls FIG. 15B). In control wild-type mice, a 532 nm laser stimulus did not elicit a response. The results presented here indicate that at the intestinal wall, duodenal enteroendocrine cells transduced luminal stimuli onto vagal neurons within milliseconds—orders of magnitude faster than previously documented.

### Example 6

### Glutamate—a Candidate Neurotransmitter

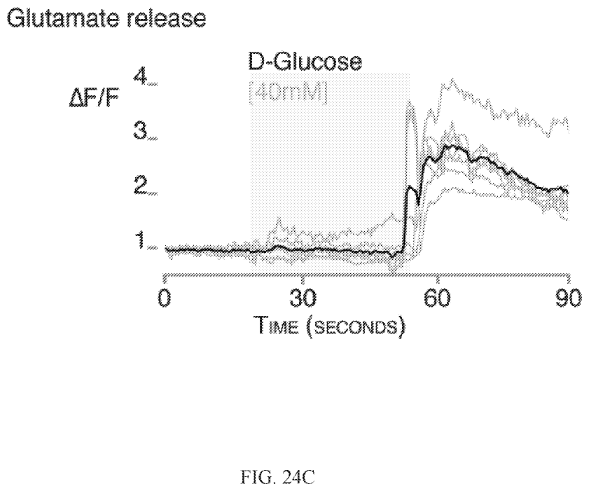

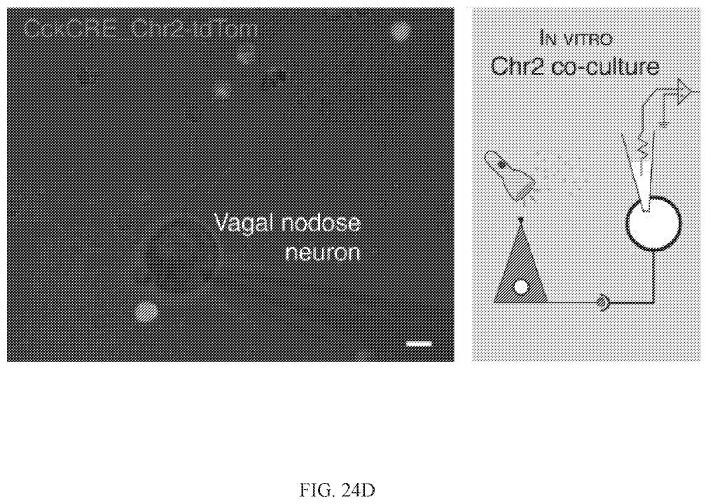

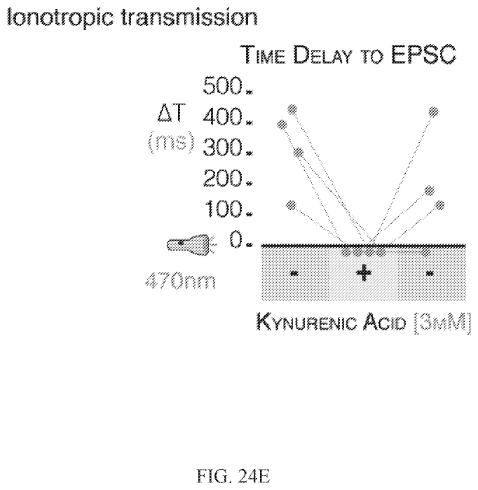

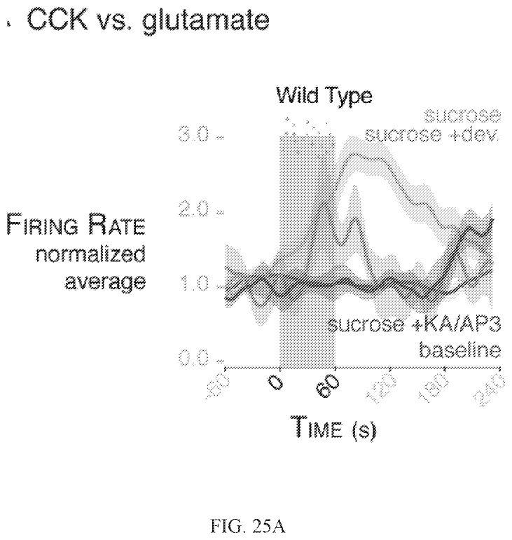

Enteroendocrine cells have historically been thought to communicate with the vagus nerve through the paracrine action of hormones, such as CCK or PYY. Their synaptic links with vagal neurons uncovered a second mechanism. One in which, luminal stimuli could be transduced up to the brain much faster than previously described. Enteroendocrine cells could also use a classic neurotransmitter to transduce such sensory signals. Intestinal enteroendocrine cells expressed significant quantities of the gene for the vesicular glutamate transporter 1 protein (Vglut1) (FIGS. 4K-4L). Moreover, vagal nodose neurons expressed at least 8 glutamatergic receptors (FIG. 15C). In a transgenic Vglut1CRE_YFP mouse, YFP fluorescence was observed in distinctive intestinal epithelial cells that resemble enteroendocrine cells. Almost 4 in 10 of those Vglut1CRE_YFP positive cells co-stained for CCK (38.80%±2.53% SEM; 100 cells per n; n=3). Vesicular glutamate released at the synaptic cleft was the major excitatory neurotransmitter in the mammalian brain. FIG. 24A shows a possible model of synaptic neurotransmission in enteroendocrine cells using glutamate. Thus, the data above suggests that glutamate could also be a neurotransmitter in enteroendocrine cells.

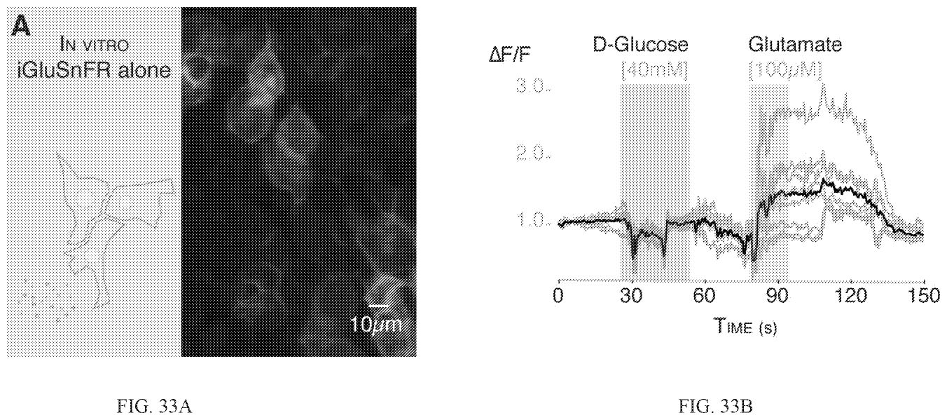

To test whether enteroendocrine cells release glutamate, the sniffer protein iGluSnFR was used. When transfected into HEK cells, iGluSnFR is membrane bound and fluoresces green in the presence of glutamate. iGluSnFR-HEK cells cultured alone did not respond to a D-glucose (40 mM) stimulus but did respond to glutamate (100 μM) (FIGS. 33A-33B). iGluSnFR-HEK cells were co-cultured with tomato expressing enteroendocrine cells (CckCRE_tdTomato) (FIG. 24B). When presented with a D-glucose stimulus (40 mM), iGluSnFR-HEK cells in close proximity to enteroendocrine cells rapidly fluoresced green (n=3 cultures; FIG. 24C), indicating that enteroendocrine cells release the neurotransmitter glutamate. In co-culture, when Chr2 enteroendocrine cells connect with nodose neurons, a 470 nm stimulus elicited EPSCs and adding the ionotropic glutamate receptor blocker kynurenic acid (3 mM) abolished the light evoked response (FIGS. 24D-24E). The response was recovered once the blocker is washed away (n=4 pairs) (FIG. 24E).

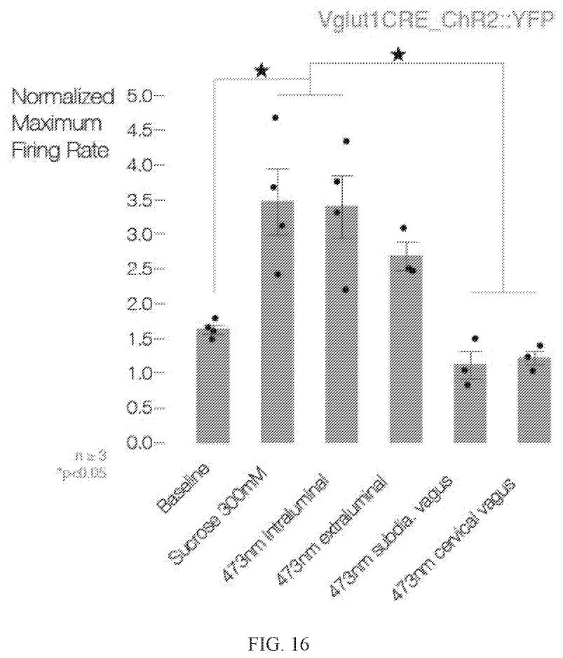

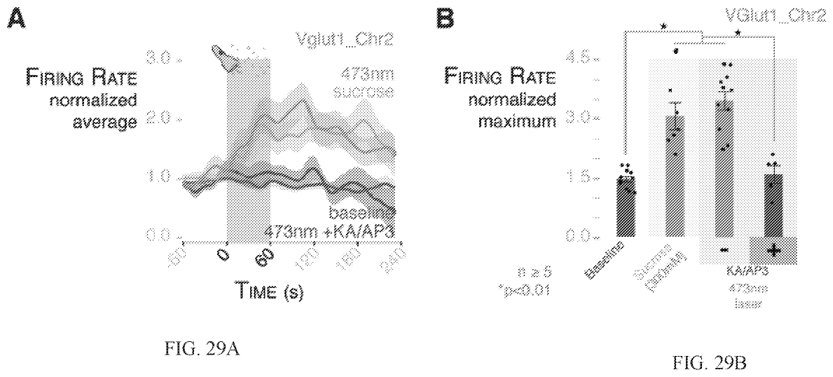

Whether activation of Vlgut1 expressing intestinal epithelial cells was sufficient to drive vagal nerve excitation was investigated using a transgenic Vglut1CRE_ChR2-YFP mouse. In these mice, 473 nm laser stimulus delivered intraluminally significantly increased vagal nerve firing from baseline (FIGS. 4M-4N). The amplitude and timing of the peak responses were comparable to results from the CckCRE_ChR2 experiments (FIGS. 4A-4D). The vagal firing rate responded rapidly to 300 mM sucrose and 473 nm intraluminal laser applications as shown by the time to peak and area under the curve (A.U.C.) (FIG. 28). To ensure that this effect was not due to direct depolarization of the vagal nodose neurons, the laser was applied to the sub-diaphragmatic and cervical vagus and found no change in firing rate from baseline (FIG. 16). Moreover, the response to 473 nm laser was abolished in the presence of a cocktail of glutamate receptor blockers (AP-3 [1 mg/kg]+Kynurenic acid [150 μg/kg]) (FIGS. 29A-29B). These data show that glutamate is a candidate neurotransmitter used by intestinal enteroendocrine cells to transduce signals onto vagal neurons.

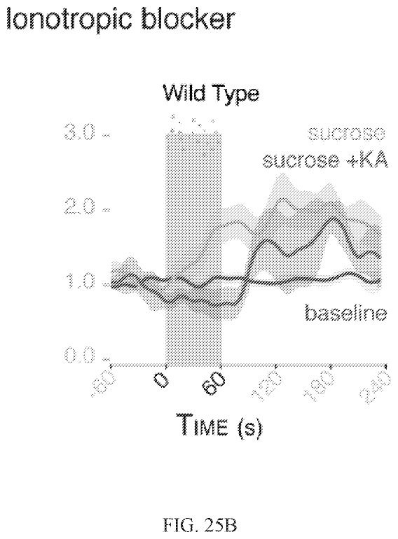

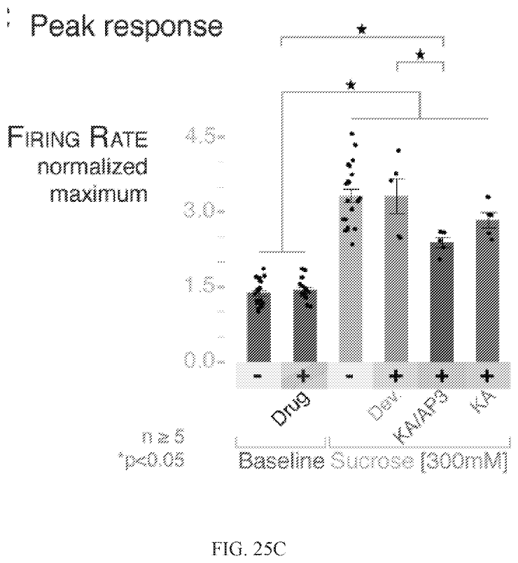

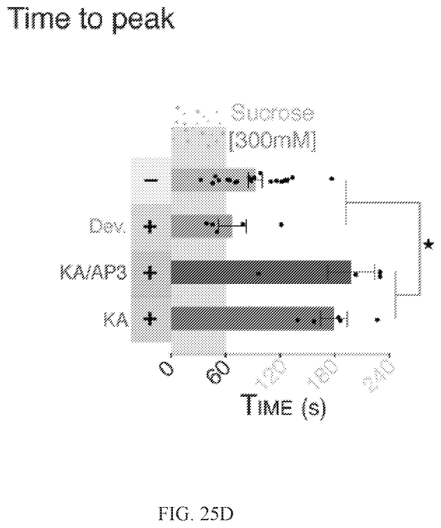

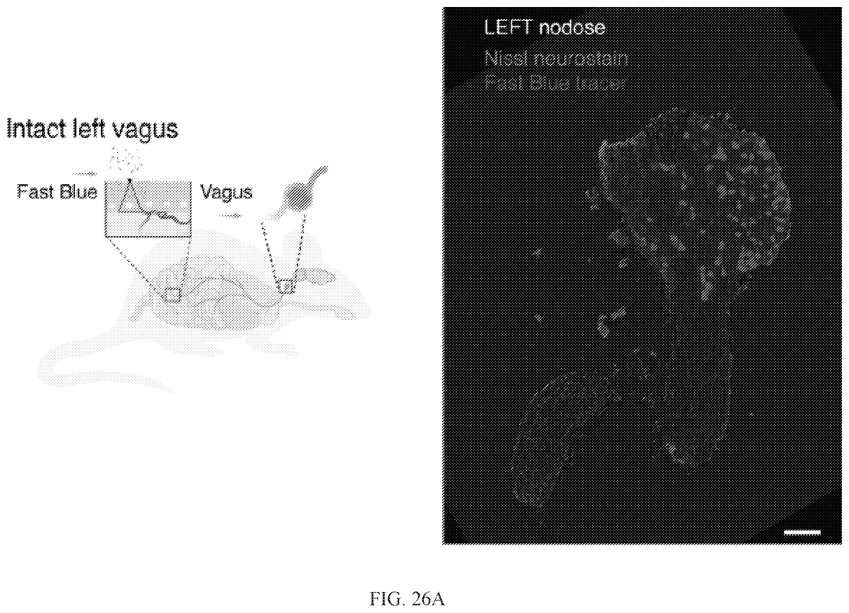

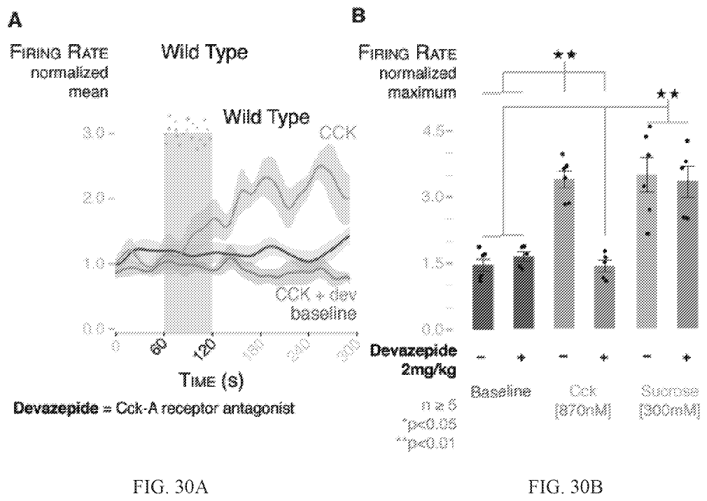

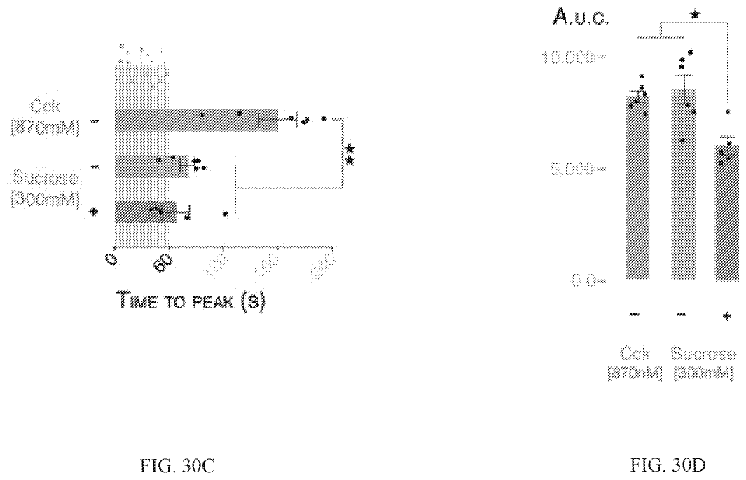

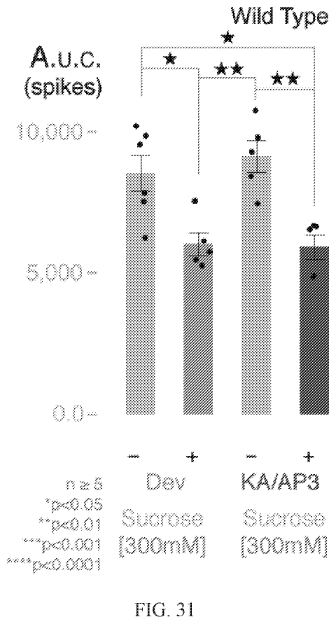

Infusing CCK8 (870 nM) into the intestinal lumen increased the vagal firing rate to a similar maximum reached by sucrose (FIGS. 30A-30B). However, the time to peak between stimuli was significantly different. The CCK response reached a maximum peak firing rate about 180 s after the onset of the stimulus, whereas the sucrose response reached a maximum peak after about 60 s (FIG. 30C). Moreover, inhibition of the CCK-A receptor with devazepide (2 mg/kg) did not affect the peak firing rate triggered by sucrose (FIG. 25C). The sucrose response was only attenuated after 120 seconds of the stimulus (FIG. 25A). However, the glutamate receptor blocker cocktail attenuated the speed, peak, and magnitude of the vagal response to sucrose (FIG. 25C-25D and FIG. 31). Indeed, the first 60 seconds of the vagal response to sucrose was suppressed by the ionotropic blocker kynurenic acid alone (FIG. 25B and FIGS. 32A-32D). Adding the ionotropic blocker was sufficient to delay the time to peak to the level of CCK alone, around 180 seconds (FIG. 25D and FIG. 30C). These data revealed fast synaptic signal transduction for gut luminal signals to the central nervous system.

### Example 7

### Bacterial Sensor Transduction Via Enteroendocrine Cell Synapses

Changes to the gastrointestinal microbiota have been linked to alterations in appetite and metabolic syndrome. Conserved bacterial components were detected by human immune cells through pattern recognition receptors such as Toll-like receptor 5 (TLR-5), which detects the bacterial ligand flagellin. Enteroendocrine cells, which sense intraluminal food contents and have direct contact with the intraluminal bacteria.

Animals

C57BL/6 mice with LoxP sites flanking Tlr5 (Jackson) were crossed with mice expressing Cre recombinase driven by PYY (Jackson) to form mice in which Toll-like receptor 5 was selectively knocked out in colonic enteroendocrine cells.

Body Weight, Activity, and Feeding Behavior Measurements

Four of the PYYCRE_TLR5KO transgenic mice were compared with four age and gender-matched (all five months old; 3 male, 1 female) LoxP-Tlr5 only mice (NegCRE_TLR5KO) in the Phenomaster System (TSE). The Phenomaster system is a home cage automated behavioral monitoring system that continuously measures body weight, activity, and feeding behavior. The mice were singly housed for two days in the Phenomaster to acclimate before measurements were taken for 3 consecutive days. Mice were given unrestricted access to normal laboratory diet food throughout both light and dark cycles. All weights (both food and body) were verified with daily weighings on a laboratory scale. Welch's two-sample t-test was used to compare means.

PYYCRE_TLR5KO Mice have a Larger Weight.

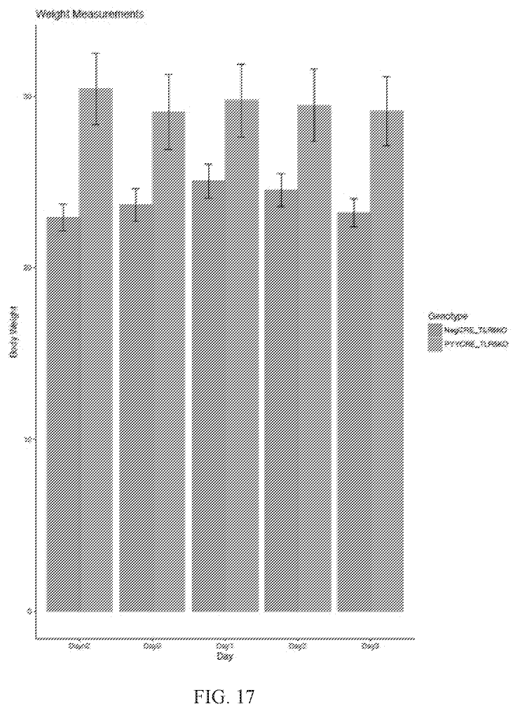

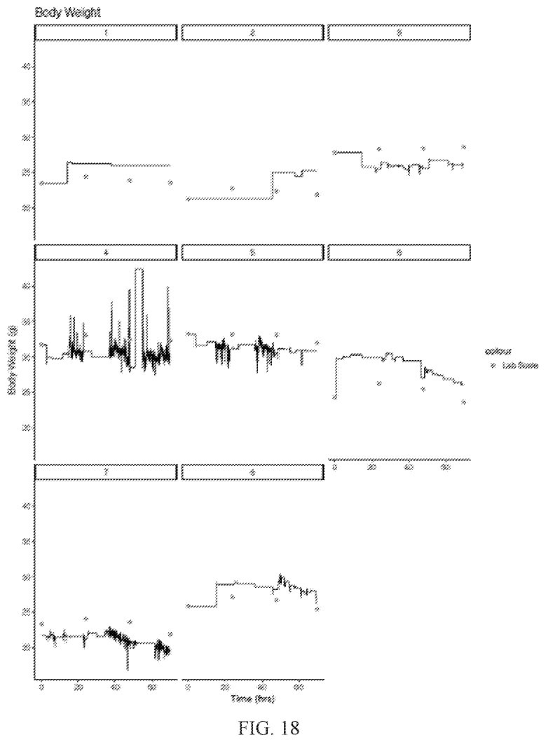

Prior to starting the measurements, the PYYCRE_TLR5KO mice weighed more than the NegCRE_TLR5KO mice (30.4 vs. 22.9; p<0.05; FIG. 17). Weights remained relatively stable across the five total days of testing. This suggests that the metabolic syndrome phenotype described in 2010 by Vijay-kumar et al. in Science may have been driven by enteroendocrine cells within the intestinal epithelial layer. Phenomaster body weight measurements did not always match lab scale measurements, and thus, lab scale measurements were used for analysis (FIG. 18).

PYYCRE_TLR5KO Consume Less Food.

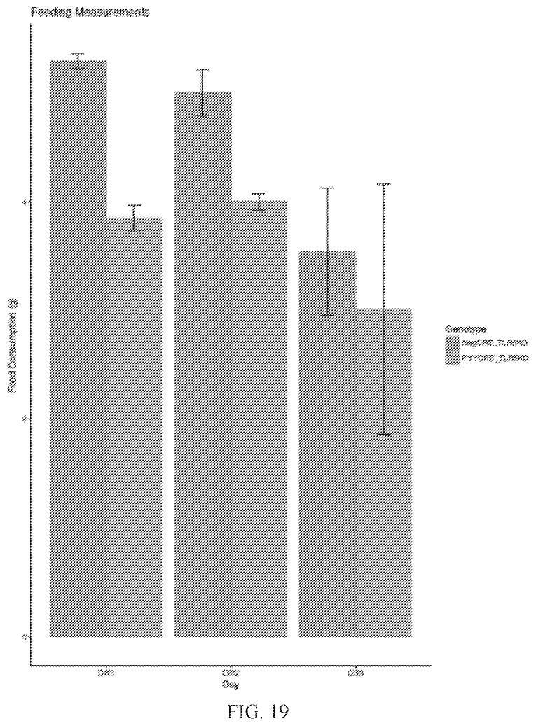

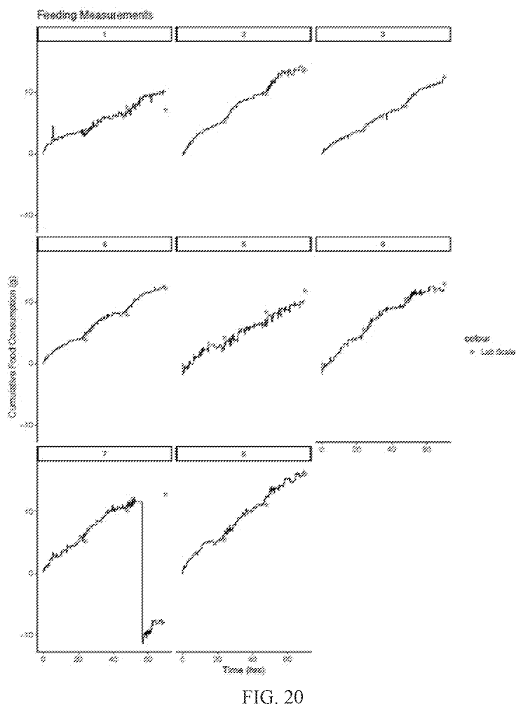

PYYCRE_TLR5KO mice tended to eat less food than the NegCRE_TLR5KO mice (FIG. 19). However, the difference shrunk as the days progressed, suggesting that the mice had not fully acclimated to the testing chamber at the time of analysis. Unfortunately, the NegCRE_TLR5KO mice were noted to be fighting and to have significant fur scratches when first retrieved from home cage, which may confound these early feeding results. Phenomaster feeding measurements closely matched lab scale measurements (FIG. 20).

### Example 8

### Autism Alters the Ability of Enteroendocrine Cells to Sense and Secrete Neuropeptide Hormones

Fluorescence in Enteroendocrine Cells of MIA Model Mice.

Behaviors related to these symptoms were present in a mouse model of an environmental risk factor for autism, maternal immune activation (MIA). MIA mice were optimized for fluorescence expression in enteroendocrine cells. Fluorescent enteroendocrine cells were isolated and single-cell PCR was completed on extracted RNA. In this animal model of autism, gut hormone genes and sensory receptors for nutrients were significantly altered compared to cells from litter mate control.

### Example 9

### Targeting a Gut Sensory Neurocircuit to Modulate Autism Spectrum Disorders

The effects of MIA on enteroendocrine cell neurotransmission was determined. The focus was to establish the Maternal Immune Activation (MIA) model of ASD and determine the effects of MIA on enteroendocrine cell neurotransmission. The dose of 20 mg/kg PolylC injected at 12E (pregnancy) to induce ASD-like behaviors causes miscarriages and dead litters. This dose also alters severely the maternal behaviors and induces cannibalism in the mother. MIA responses depended on the presence of segmented filamentous bacteria (SBF) in the mouse GI. SBF was necessary for innate immune cells to mature and respond to the MIA challenge by showing the expected behavioral phenotype.

Transgenic mice that did not have SBF from JAX® lines and mice from Taconic® that did harbor SBF were used. SBF transfer took place by co-housing these strains before continuing with the behavioral phenotyping. In the MIA mice, 6 controls and 6 treated mice were used to conduct the sequencing studies. The approach was refined to obtain gene expression data from individual enteroendocrine cells. It is evident that each one of these cells has a distinct expression of genes based on their location. Based on this premise, a protocol was optimized to isolate and obtain RNA from individual cells.

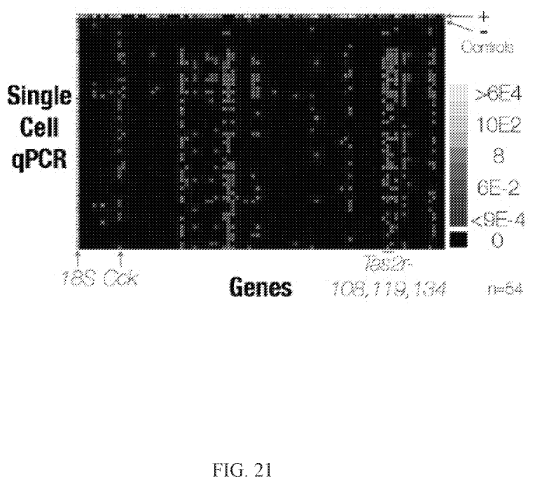

The optimization was performed because the cell size and buoyancy affects the function of the microfluidics device used to separate individual cells for genomic analysis. Data was acquired from single cells indicating that it was viable to obtain total RNA for RT-PCR and sequencing analysis. FIG. 21 shows results indicating the viability of the assay

Viable RNA will be extracted from enteroendocrine cells dissociated from MIA animals vs. controls to perform single cell sequencing.

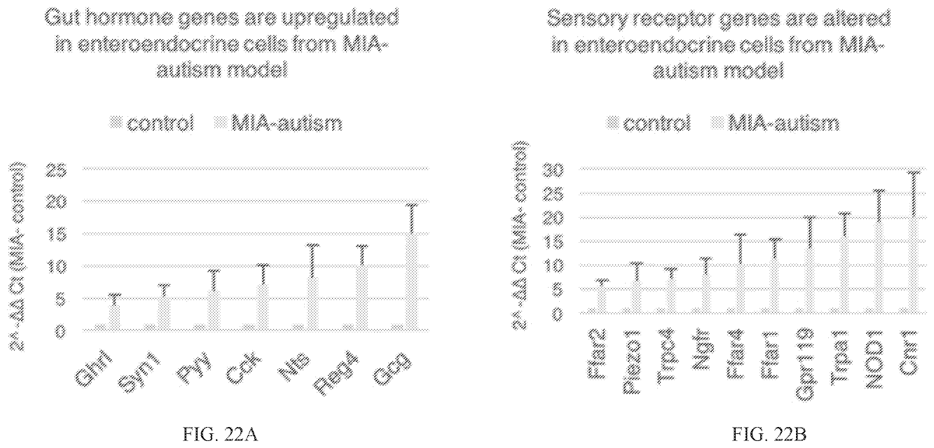

Enteroendocrine cells were purified from MIA mice and controls to test the expression of several different genes encoding for nutrient-sensing receptors as well as gut hormones in a population of enteroendocrine cells (as supposed to individual cells). As shown in FIGS. 22A and 22B, MIA enteroendocrine cells had a consistent increase in the expression of sensory receptors, compared to controls. Some of these included the free fatty acid receptors Ffar1 and Ffar4, the cannabinoid receptor Gpr119 that regulates satiety, the bacterial-ligand receptor Nod1, and the Trpa1 ion channel—a receptor for irritants that give rise to pain or itch. MIA enteroendocrine cells also showed a consistent upregulation of several gut hormones that regulate hunger and satiety, including Peptide YY (Pyy), Neurotensin (Nts), Cholecystokinin (Cck), and Ghrelin (Ghrl). These data suggest that in the MIA mouse model of ASD enteroendocrine cells were hypersensitive to sensory stimuli.

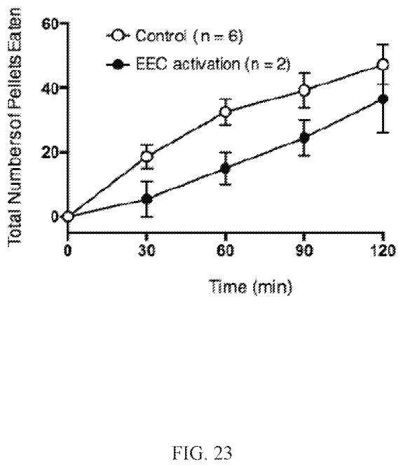

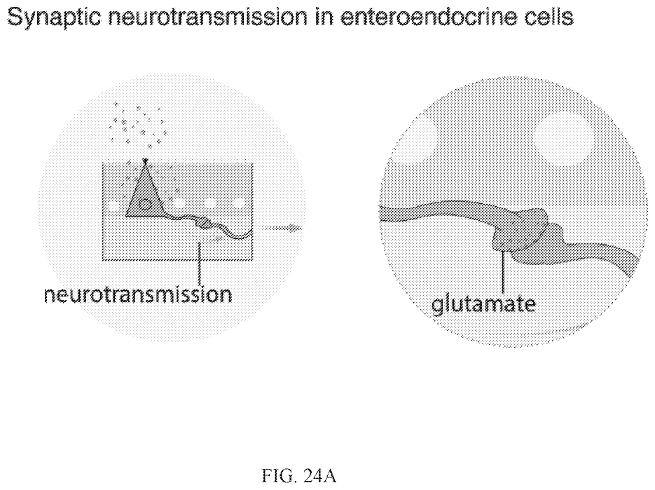

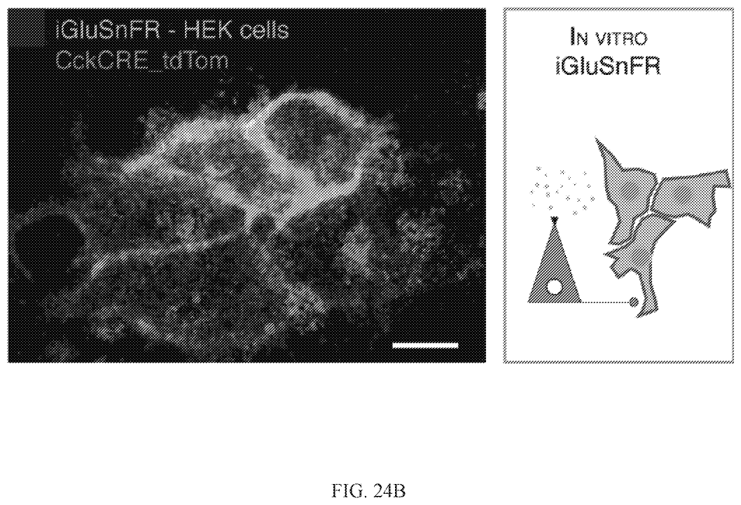

Enteroendocrine cell photostimulation was tested to determine if it was sufficient to alter food intake in normal mice. Photostimulation of a mouse expressing in enteroendocrine cells (Cck) a light activated ion channel was performed to determine effects on food intake. The following paradigm was used: Adult (8 weeks old) CckCRE_Chr2 mice (n=2) and aged matched wild type controls (n=6) were fitted with a window implant. Three days later and upon recovery the mice were fasted for 12 hours. Mice were then photo-stimulated through the window implant with a laser pulse of 50 Hz for 1 h. At the end of it, mice were presented with pelleted mouse chow. The results (FIG. 23) show that photo-stimulation of enteroendocrine cells results in reduced appetitive behavior. The results are maintained up to 2 hours post-photostimulation.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

For reasons of completeness, various aspects of the disclosure are set out in the following numbered clauses:

Clause 1. A method of modulating a transsynaptic signal through a neuroepithelial circuit between a gut sensory cell and the brain, the method comprising stimulating or inhibiting a receptor on the gut sensory cell, thereby stimulating or inhibiting the transsynaptic signal from the gut sensory cell to the brain.

Clause 2. A method of modulating a caloric value of a nutrient to a subject, the method comprising modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain and modulating the caloric value of the nutrient to the subject.

Clause 3. The method of clause 2, wherein the caloric value of the nutrient to the subject is increased.

Clause 4. The method of clause 2, wherein the caloric value of the nutrient to the subject is decreased.

Clause 5. The method of any one of clauses 2-4, wherein the nutrient comprises a carbohydrate, an amino acid, a protein, a fatty acid, a fat, or combinations thereof.

Clause 6. The method of clause 5, wherein the carbohydrate comprises a sugar, a starch, or a cellulose.

Clause 7. A method of modulating a bacterial stimulus signal in a subject, the method comprising modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain and modulating the bacterial stimulus signal in the subject.

Clause 8. The method of clause 7, wherein the bacterial stimulus signal in the subject is increased.

Clause 9. The method of clause 7, wherein the bacterial stimulus signal in the subject is decreased.

Clause 10. A method of modulating food intake behavior and/or food preference in a subject, the method comprising modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the food intake and/or food preference behavior in a subject is modulated.

Clause 11. The method of clause 10, wherein the food intake behavior and/or food preference in the subject is increased.

Clause 12. The method of clause 10, wherein the food intake behavior and/or food preference in the subject is decreased.

Clause 13. A method of treating a subject having or suspected of having an eating disorder, the method comprising modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein eating behavior of the subject is modulated and the subject is treated.

Clause 14. The method of clause 13, wherein the eating behavior of the subject is increased.

Clause 15. The method of clause 13, wherein the eating behavior of the subject is decreased.

Clause 16. A method of modulating anxiety in a subject, the method comprising modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the anxiety of the subject is modulated.

Clause 17. The method of clause 16, wherein the anxiety of the subject is increased.

Clause 18. The method of clause 16, wherein the anxiety of the subject is decreased.

Clause 19. A method of treating autism spectrum disorders in a subject, the method comprising modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the eating behavior of the subject is modulated and the subject is treated for autism.

Clause 20. The method of clause 19, wherein the eating behavior of the subject is increased.

Clause 21. The method of clause 19, wherein the eating behavior of the subject is decreased.

Clause 22. The method of any one of clauses 1-21, wherein the receptor is a sugar-sensing receptor, an amino acid-sensing receptor, a fatty acid-sensing receptor, and/or a bacteria-sensing receptor.

Clause 23. The method of clause 22, wherein the receptor comprises a sodium-dependent glucose cotransporter (SGLT), a taste receptor type receptor (TAS), a free fatty acid receptor (FFAR), a G-protein coupled receptor (GPR), a Toll-like receptor (TLR), a nucleotide-binding oligomerization domain-containing protein receptor (NOD), or a combination thereof.

Clause 24. The method of clause 22 or 23, wherein the receptor is SGLT1 or TLR5.

Clause 25. The method of any one of clauses 1-24, wherein stimulating or inhibiting the receptor on the gut sensory cell comprises contacting the gut sensory cell with a composition capable of stimulating or inhibiting the receptor on the gut sensory cell.

Clause 26. The method of any one of clauses 2-24, wherein the stimulating or inhibiting the receptor on the gut sensory cell comprises administering to the subject a therapeutically effective amount of a composition capable of stimulating or inhibiting the receptor on the gut sensory cell of the subject

Clause 27. The method of clause 25 or 26, wherein the composition comprises a modulator of the receptor.

Clause 28. The method of clause 27, wherein the modulator of the receptor comprises an agonist of the receptor, an antagonist of the receptor, and/or an inhibitor of the receptor.

Clause 29. The method of clause 28, wherein the modulator of the receptor comprises an SGLT1 inhibitor.

Clause 30. The method of clause 29, wherein the SGLT1 inhibitor is phloridzin dehydrate.

Clause 31. The method of any one of clauses 25-30, wherein the composition comprises a sugar, an amino acid, a fatty acid, a bacteria, or a molecule derived from a bacteria.

Clause 32. The method of clause 31, wherein the sugar comprises glucose, sucrose, fructose, sucralose, or a combination thereof.

Clause 33. The method of clause 31 or 32, wherein the composition comprises between about 15 mM and about 300 mM of sugar.

Clause 34. The method of any one of clauses 31-33, wherein the composition comprises at least about 100 mM sugar.

Clause 35. The method of any one of clauses 31-34, wherein the composition comprises at least about 300 mM sucrose.

Clause 36. The method of any one of clauses 31-35, wherein the composition comprises at least about 15 mM of sucralose.

Clause 37. The method of any one of clauses 25-36, wherein the composition comprises a probiotic or a mixture of probiotics.

Clause 38. The method of any one of clauses 1-37, wherein the neuroepithelial circuit comprises a nerve fiber.

Clause 39. The method of clause 38, wherein the neuroepithelial circuit comprises a gut sensory cell in contact with the nerve fiber.

Clause 40. The method of clause 38 or 39, wherein the gut sensory cell is in contact with the nerve fiber by releasing a neurotransmitter.

Clause 41. The method of clause 40, wherein the neurotransmitter is glutamate.

Clause 42. The method of any one of clauses 38-41, wherein the nerve fiber is a vagal nerve fiber or a sensory nerve fiber.

Clause 43. The method of clause 42, wherein the vagal nerve fiber includes a vagal nodose neuron.

Clause 44. The method of any one of clauses 1-43, wherein the gut sensory cell comprises a gut epithelial cell.

Clause 45. The method of clause 1-44, wherein the gut sensory cell comprises an enteroendocrine cell and/or an enterochromaffin cell.

Clause 46. The method of clause 45, wherein the neuroepithelial circuit comprises an enteroendocrine cell in contact with a vagal nerve fiber.

Clause 47. The method of any one of clauses 2-46, wherein the subject is a human subject.

Clause 48. The method of any one of clauses 1-47, further comprising stimulating or inhibiting a receptor on a nerve fiber.

Clause 49. A method of modulating a transsynaptic signal through a neuroepithelial circuit between a gut sensory cell and the brain, the method comprising stimulating or inhibiting a receptor on a nerve fiber, thereby stimulating or inhibiting the communication of the gut sensory cell with the nerve fiber.

Clause 50. The method of clause 48 or 49, wherein stimulating or inhibiting the receptor on the nerve fiber comprises contacting the nerve fiber with a composition capable of stimulating or inhibiting a neurotransmitter released by the gut sensory cell.

Clause 51. The method of clause 50, wherein the composition inhibits the neurotransmitter from binding to a receptor on the nerve fiber, thereby inhibiting the communication of the gut sensory cell with the nerve fiber.

Clause 52. The method of clause 50 or 51, wherein the neurotransmitter is glutamate.

Clause 53. The method of clause 52, wherein the receptor on the nerve fiber is an ionotropic glutamate receptor.

Clause 54. The method of clause 53, wherein the receptor on the nerve fiber is an N-methyl-D-aspartate (NMDA) receptor, an α-amino-3-hydroxy-5-methyl-4-isoxazolepropionic acid (AMPA) receptor or a kainate receptor.

Clause 55. The method of any one of clauses 50-54, wherein the composition inhibiting the neurotransmitter comprises kynurenic acid or DL-2-amino-3-phosphonopropionic acid (AP-3).

Clause 56. The method of clause 50 or 51, wherein the neurotransmitter is Cholecystokinin (CCK).

Clause 57. The method of clause 56, wherein the receptor on the nerve fiber is a CCK-A receptor or a CCK-B receptor.

Clause 58. The method of clause 57, wherein the composition inhibiting the neurotransmitter comprises devazepide.

Clause 59. The method of any one of clauses 49-58, wherein the nerve fiber is a vagal nerve fiber or a sensory nerve fiber.

Clause 60. The method of clause 59, wherein the vagal nerve fiber includes a vagal nodose neuron.

Clause 61. The method of any one of clauses 49-60, wherein the gut sensory cell comprises a gut epithelial cell.

Clause 62. The method of clause 49-61, wherein the gut sensory cell comprises an enteroendocrine cell and/or an enterochromaffin cell.

Clause 63. The method of any one of clauses 49-62, wherein the neuroepithelial circuit comprises an enteroendocrine cell in contact with a vagal nerve fiber.

1. A method of modulating a transsynaptic signal through a neuroepithelial circuit between a gut sensory cell and the brain, the method comprising stimulating or inhibiting a receptor on the gut sensory cell, thereby stimulating or inhibiting the transsynaptic signal from the gut sensory cell to the brain.

2. A method of modulating a caloric value of a nutrient to a subject, the method comprising modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain and modulating the caloric value of the nutrient to the subject.

3-9. (canceled)

10. A method of modulating food intake behavior and/or food preference in a subject, the method comprising modulating a transsynaptic signal through a neuroepithelial circuit between a gut sensory cell and the brain by stimulating or inhibiting a receptor on the gut sensory cell, thereby modulating the transsynaptic signal from the gut sensory cell to the brain, wherein the food intake and/or food preference behavior in a subject is modulated.

11-21. (canceled)

22. The method of claim 1, wherein the receptor is a sugar-sensing receptor, an amino acid-sensing receptor, a fatty acid-sensing receptor, and/or a bacteria-sensing receptor.

23. The method of claim 22, wherein the receptor comprises a sodium-dependent glucose cotransporter (SGLT), a taste receptor type receptor (TAS), a free fatty acid receptor (FFAR), a G-protein coupled receptor (GPR), a Toll-like receptor (TLR), a nucleotide-binding oligomerization domain-containing protein receptor (NOD), or a combination thereof.

24. The method of claim 22, wherein the receptor is SGLT1 or TLR5.

25. The method of claim 1, wherein stimulating or inhibiting the receptor on the gut sensory cell comprises contacting the gut sensory cell with a composition comprising a modulator of the receptor capable of stimulating or inhibiting the receptor on the gut sensory cell.

26-28. (canceled)

29. The method of claim 25, wherein the modulator of the receptor comprises an SGLT1 inhibitor.

30. The method of claim 29, wherein the SGLT1 inhibitor is phloridzin dehydrate.

31. The method of claim 25, wherein the composition comprises a sugar, an amino acid, a fatty acid, bacteria, or a molecule derived from a bacteria.

32. The method of claim 31, wherein the sugar comprises glucose, sucrose, fructose, sucralose, or a combination thereof.

33. The method of claim 31, wherein the composition comprises between about 15 mM and about 300 mM of sugar.

34. The method of claim 31, wherein the composition comprises at least about 100 mM sugar.

35-36. (canceled)

37. The method of claim 25, wherein the composition comprises a probiotic or a mixture of probiotics.

38. (canceled)

39. The method of claim 1, wherein the neuroepithelial circuit comprises a gut sensory cell in contact with a nerve fiber by releasing a neurotransmitter.

40. (canceled)

41. The method of claim 39, wherein the neurotransmitter is glutamate.

42. The method of claim 39, wherein the nerve fiber is a vagal nerve fiber or a sensory nerve fiber.

43. The method of claim 42, wherein the vagal nerve fiber includes a vagal nodose neuron.

44. (canceled)

45. The method of claim 1, wherein the gut sensory cell comprises a gut epithelial cell, an enteroendocrine cell, and/or an enterochromaffin cell.

46. The method of claim 45, wherein the neuroepithelial circuit comprises an enteroendocrine cell in contact with a vagal nerve fiber.

47-63. (canceled)
